SEQUENCE LISTING (1 sequence)

SEQ ID NO: 1 - protein, 20 aa, Artificial Sequence
  Description of Artificial Sequence Synthetic peptide
AAVALLPAVLLAALAPIETD

(19) United States
(12) Patent Application Publication
(10) Pub. No.: US 2021/0077562 A1
(43) Pub. Date: Mar. 18, 2021

(54) ETBR ANTAGONIST COMPOUNDS, COMPOSITIONS, AND USES

(71) Applicant: ENB Therapeutics, Inc., New York, NY (US)

(72) Inventor: Sumayah JAMAL, New York, NY (US)

(21) Appl. No.: 17/101,676

(22) Filed: Nov. 23, 2020

Related U.S. Application Data
(63) Continuation of application No. 16/419,931, filed on May 22, 2019.
Application No. 16/419,931 is a continuation of application No. PCT/US2019/025050, filed on Mar. 29, 2019.
(60) Provisional application No. 62/650,477, filed on Mar. 30, 2018.

(51) Int. Cl. A61K 38/06; A61P 35/00; A61P 35/04; C07K 16/28; A61K 39/395; A61K 9/51

(52) U.S. Cl. CPC A61K 38/06; A61P 35/00; A61P 35/04; A61K 2039/505; A61K 39/39558; A61K 9/51; C07K 16/2818

(57) ABSTRACT

Disclosed herein are ETBR antagonist compounds, pharmaceutical compositions thereof, methods for treating cancers, and methods of forming tertiary lymphoid organs.

### CROSS-REFERENCE

This application is a continuation of U.S. application Ser. No. 16/419,931, filed May 22, 2019, which is a continuation of International Application No. PCT/US2019/025050, filed Mar. 29, 2019, which claims the benefit of U.S. Provisional Application No. 62/650,477, filed Mar. 30, 2018, each of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Nov. 18, 2020, is named “ENB_004C1_SL.txt” and is 685 bytes in size.

### BRIEF SUMMARY

Disclosed herein is a method of forming a tertiary lymphoid organ (TLO) in a subject in need thereof, comprising administering to the subject an Endothelin B receptor (ETBR) antagonist. In some embodiments, the ETBR antagonist is BQ-788, A192621, A-308165, IRL-1038, IRL-2500, RO-468443, BQ-017, or a structural analog thereof. In some embodiments, the ETBR antagonist is in a form of nanoparticles. In some embodiments, the ETBR antagonist is formulated as a controlled, or delayed release formulation. In certain embodiments, the ETBR antagonist is formulated as nanoparticles. In some embodiments, the ETBR antagonist is a non-deuterated BQ-788 analog. In some embodiments, the method further comprises administering to the subject at least one additional therapeutic agent.

The formation of a TLO in a subject is beneficial for the treatment of diseases including infection by microbes, graft rejection in transplantation medicine, cancers, autoimmune disorders and autoimmune related conditions. In embodiments described herein, one or more of these conditions can be treated in a subject by forming TLOs in the subject. In certain cases, TLOs are formed by administering an ETBR antagonist. On some other cases, TLOs are formed by administering an ETBR antagonist in combination with one or more additional therapeutic agents.

In certain cases, the additional therapeutic agent is an anti-oncologic therapeutic agent, an anti-bacterial or an antimicrobial therapeutic agent. In some cases, the additional therapeutic agent is an agent used to reduce transplant rejection such as an immune suppressant or an anti-CD40 agent.

In some embodiments, the one anti-oncologic agent comprises a bRAF inhibitor, an immune checkpoint inhibitor, a caspase-8 inhibitor, an ETAR antagonist, niacinamide, a chemotherapeutic agent, or any combination thereof. In some embodiments, the anti-oncologic agent comprises at least one of the immune checkpoint inhibitor. In some embodiments, the immune checkpoint inhibitor comprises at least one anti-PD1 antibody, at least one anti-PD-L1 antibody, at least one anti-CTLA4 antibody, or any combination thereof. In some embodiments, the at least one anti-PD1 antibody comprises pidilizumab, BMS-936559, nivolumab, pembrolizumab, or any combination thereof. In some embodiments, the at least one anti-PD-L1 antibody comprises atezolizumab, avelumab, durvalumab, MDX-1105, or any combination thereof. In some embodiments, the ETBR antagonist and the at least one additional therapeutic agent are administered at different times. In some embodiments, the ETBR antagonist is administered at 2, 3, 4, or 5 times the frequency of the additional therapeutic agent, for instance immune checkpoint inhibitor. In some embodiments, ETBR antagonist is administered 3 times frequently as the immune checkpoint inhibitor. In some embodiments, the ETBR antagonist is administered 3 times every 2-3 weeks and the immune checkpoint inhibitor is administered 1 time about every 2-3 weeks. In some embodiments, the compound is administered 3 times about every 21 days and the immune checkpoint inhibitor is administered 1 time about every 21 days.

In some embodiments, the tertiary lymphoid organ is formed within, or adjacent to a tumor, for example a solid tumor, melanoma tumor, solid tumor cancer, malignant melanoma, metastatic melanoma, malignant squamous cell carcinoma, metastatic squamous cell carcinoma, glioblastoma, brain cancer, pancreatic cancer, colon cancer, breast cancer, ovarian cancer, prostate cancer, or any combination thereof. In some embodiments, the subject is a human. In some embodiments, the subject is resistant to an immunotherapy before the treatment. In some embodiments, the administration restores Tumor Infiltrating Lymphocytes (TILs) in a tumor microenvironment.

Also disclosed herein are compounds for use as ETBR antagonists and/or for TLO formation. In some embodiments, disclosed herein is a compound of Formula (4):

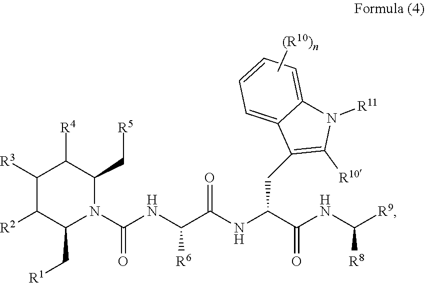

a stereoisomer thereof, or a pharmaceutically acceptable salt or solvate thereof,
wherein,
each of R¹-R⁵ is independently hydrogen, deuterium, halogen, hydroxy, amino, nitro, cyano, C₁-C₈ alkyl, C₂-C₈ alkenyl, C₂-C₈ alkynyl, C₃-C₈ cycloalkyl, C₁-C₈ alkoxy, C₁-C₈ haloalkyl, aryl, or heteroaryl;
R⁶ is C₁-C₈ alkyl, C₂-C₈ alkenyl, C₂-C₈ alkynyl, C₃-C₈ cycloalkyl, C₁-C₈ alkoxy, C₁-C₈ haloalkyl, aryl, or heteroaryl, wherein R⁶ optionally comprises deuterium;
R⁸ and R⁹ are each independently C₁-C₈ alkyl, C₂-C₈ alkenyl, C₂-C₈ alkynyl, C₃-C₈ cycloalkyl, C₁-C₈ alkoxy, C₁-C₈ haloalkyl, aryl, heteroaryl, or —COOR′, or R⁸ and R⁹ may be taken together to form substituted or unsubstituted C₃-C₈ cycloalkyl, substituted or unsubstituted C₂-C₇ heterocycloalkyl, substituted or unsubstituted aryl, substituted or unsubstituted heteroaryl, or substituted or unsubstituted polycyclic ring system, wherein R⁸ or R⁹ each optionally comprises deuterium;
R¹⁰ and R^{10′} are each independently hydrogen, deuterium, halogen, hydroxy, amino, nitro, cyano, C₁-C₈ alkyl, C₂-C₈ alkenyl, C₂-C₈ alkynyl, C₃-C₈ cycloalkyl, C₁-C₈ alkoxy, C₁-C₈ haloalkyl, aryl, or heteroaryl;
R¹¹ is hydrogen, deuterium, C₁-C₈ alkyl, C₁-C₈ haloalkyl, C₃-C₈ cycloalkyl, aryl, heteroaryl, or —COOR′;
each R′ is independently hydrogen or C₁-C₈ alkyl; and
n is an integer from 0-4.
   In some embodiments, R², R³, and R⁴ are hydrogen, and wherein R¹ and R⁵ are methyl. In some embodiments, R⁶ is —(CH₂)C(CH₃)₃. In some embodiments, R¹⁰ and R^{10′} are hydrogen, and wherein R¹¹ is —COOCH₃. In some embodiments, R⁸ is —(CH₂)₃CH₃. In some embodiments, the compound is selected from the group consisting of:

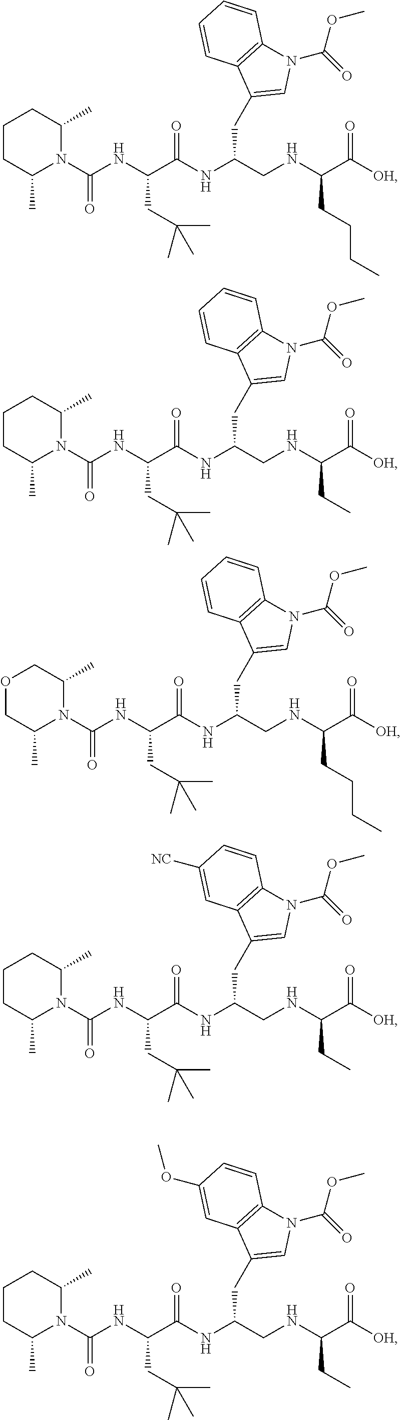

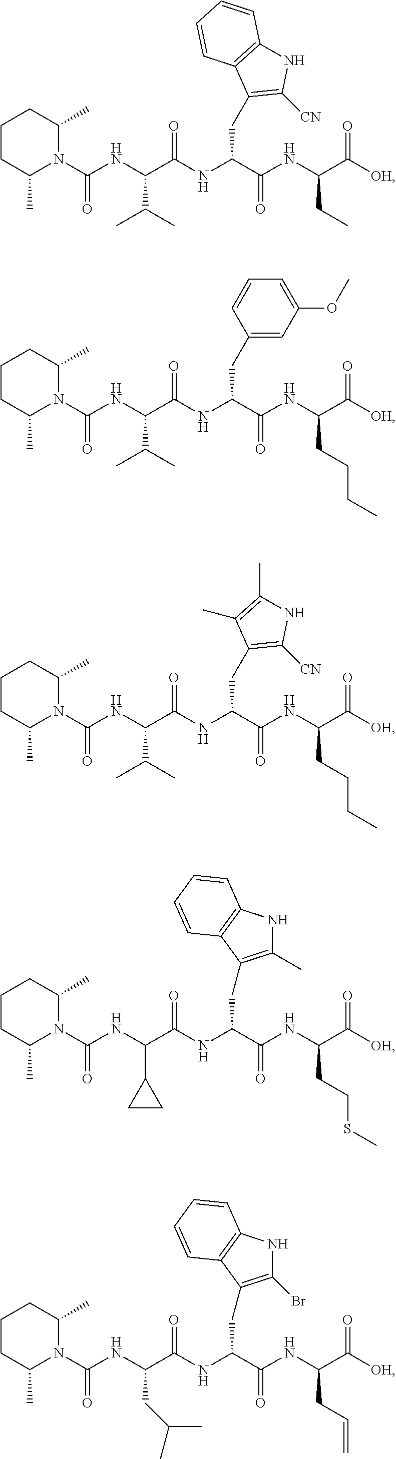 [SMILES: CC[C@@H](NC(=O)[C@@H](Cc1c(C#N)[nH]c2ccccc12)NC(=O)[C@@H](NC(=O)N1[C@H](C)CCC[C@@H]1C)C(C)C)C(=O)O ; CCCC[C@@H](NC(=O)[C@@H](Cc1cccc(OC)c1)NC(=O)[C@@H](NC(=O)N1[C@H](C)CCC[C@@H]1C)C(C)C)C(=O)O ; CCCC[C@@H](NC(=O)[C@@H](Cc1c(C#N)[nH]c(C)c1C)NC(=O)[C@@H](NC(=O)N1[C@H](C)CCC[C@@H]1C)C(C)C)C(=O)O ; CSCC[C@@H](NC(=O)[C@@H](Cc1c(C)[nH]c2ccccc12)NC(=O)C(NC(=O)N1[C@H](C)CCC[C@@H]1C)C1CC1)C(=O)O ; C=CC[C@@H](NC(=O)[C@@H](Cc1c(Br)[nH]c2ccccc12)NC(=O)[C@H](CC(C)C)NC(=O)N1[C@H](C)CCC[C@@H]1C)C(=O)O]

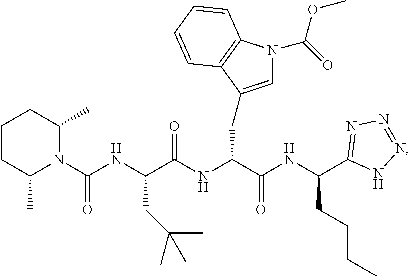 [SMILES: CCCC[C@@H](NC(=O)[C@@H](Cc1cn(C(=O)OC)c2ccccc12)NC(=O)[C@H](CC(C)(C)C)NC(=O)N1[C@H](C)CCC[C@@H]1C)c1nnn[nH]1]

a stereoisomer thereof, a deuterated analog thereof, a fluorinated analog thereof, and a pharmaceutically acceptable salt or solvate thereof.

Also disclosed herein is a compound of Formula (5):

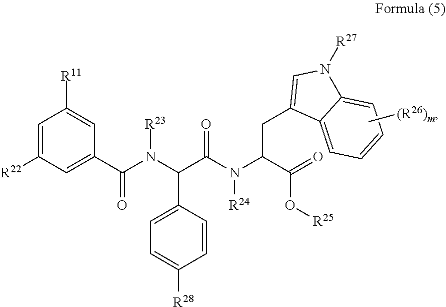

or a pharmaceutically acceptable salt or solvate thereof,
wherein,
each R²¹ and R²² is independently hydrogen, deuterium, halogen, hydroxy, amino, nitro, cyano, C₁-C₆ alkyl, C₃-C₈ cycloalkyl, or C₁-C₆ alkoxy;
each R²³ and R²⁴ is independently hydrogen or C₁-C₄ alkyl;
R²⁵ is hydrogen or C₁-C₆ alkyl;
each R²⁶ is independently deuterium, halogen, hydroxy, amino, nitro, cyano, C₁-C₆ alkyl, C₂-C₆ alkenyl, C₂-C₆ alkynyl, C₃-C₈ cycloalkyl, C₁-C₆ alkoxy, C₁-C₆ haloalkyl, aryl, or heteroaryl;
R²⁷ is hydrogen, deuterium, C₁-C₆ alkyl, C₁-C₆ haloalkyl, C₃-C₈ cycloalkyl, aryl, heteroaryl, or —COOR²⁹;
R²⁸ is substituted or unsubstituted C₃-C₈ cycloalkyl, substituted or unsubstituted C₂-C₇ heterocycloalkyl, substituted or unsubstituted aryl, or substituted or unsubstituted heteroaryl;
R²⁹ is hydrogen or C₁-C₆ alkyl; and
m is an integer from 0-4.
   In some embodiments, the compound is selected from the group consisting of

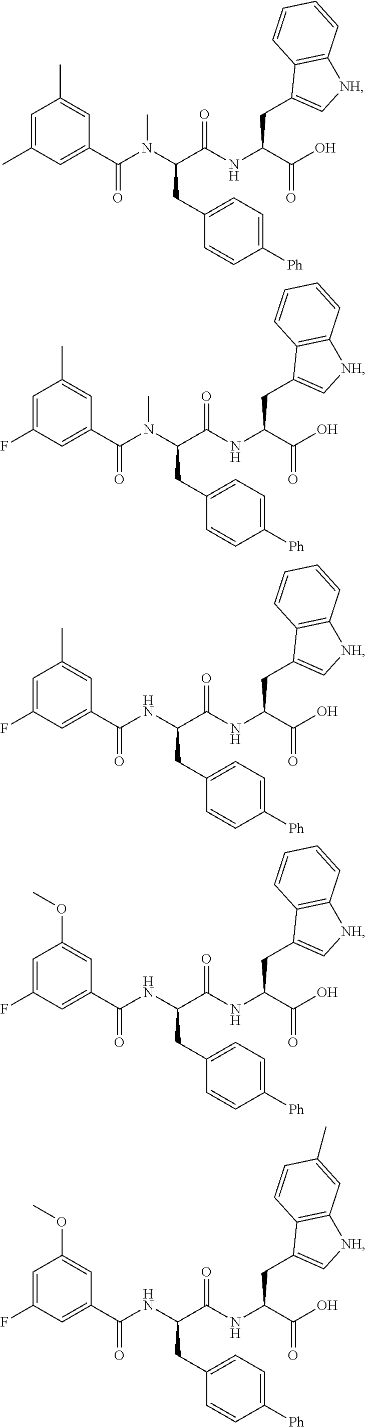

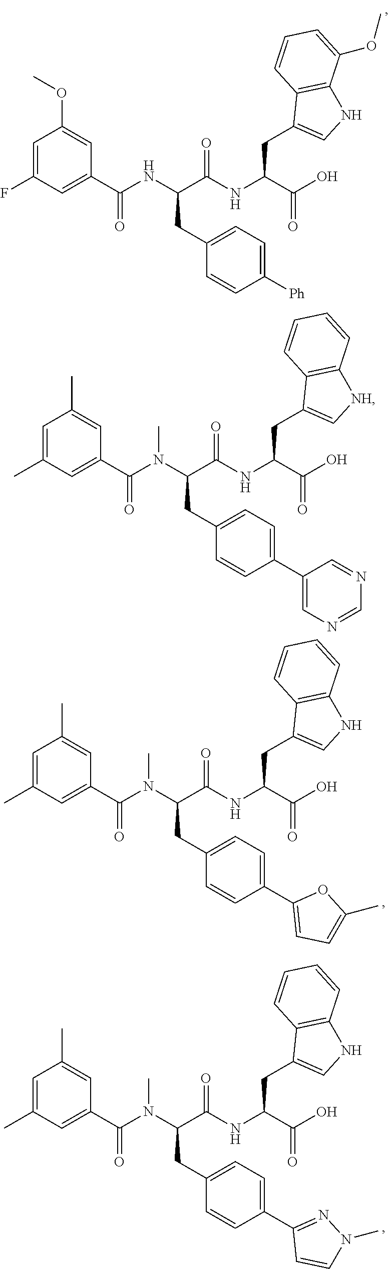

a stereoisomer thereof, a deuterated analog thereof, a fluorinated analog thereof, and a pharmaceutically acceptable salt or solvate thereof.

Also disclosed herein is a compound of Formula (6):

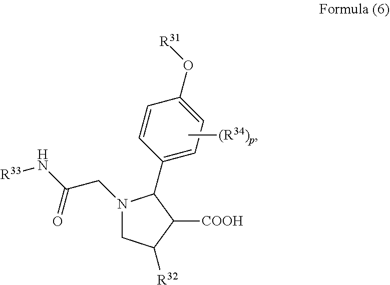

or a pharmaceutically acceptable salt or solvate thereof,
wherein,
R³¹ is substituted or unsubstituted C₁-C₆ alkyl, substituted or unsubstituted C₃-C₈ cycloalkyl, or substituted or unsubstituted C₂-C₇ heterocycloalkyl; wherein if R³¹ is substituted then it is substituted with 1, 2, or 3 substituents independently selected from fluoro, hydroxy, amino, —NH(C₁-C₄ alkyl), —N(C₁-C₄ alkyl)₂, nitro, cyano, C₁-C₄ alkyl, and C₁-C₄ alkoxy;
R³² is substituted or unsubstituted cycloalkyl, substituted or unsubstituted heterocycloalkyl, substituted or unsubstituted aryl, or substituted or unsubstituted heteroaryl;
R³³ is substituted or unsubstituted cycloalkyl, substituted or unsubstituted heterocycloalkyl, substituted or unsubstituted aryl, substituted or unsubstituted heteroaryl, or —CH(CR³⁵)₂, wherein each R³⁵ is independently substituted or unsubstituted C₁-C₆ alkyl, substituted or unsubstituted C₃-C₈ cycloalkyl, substituted or unsubstituted C₂-C₇ heterocycloalkyl, substituted or unsubstituted aryl, or substituted or unsubstituted heteroaryl; wherein if R³³ or R³⁵ is substituted then it is substituted with 1, 2, or 3 substituents independently selected from fluoro, hydroxy, amino, —NH(C₁-C₄ alkyl), —N(C₁-C₄ alkyl)₂, nitro, cyano, C₁-C₄ alkyl, and C₁-C₄ alkoxy;
each R³⁴ is independently deuterium, halogen, hydroxy, amino, nitro, cyano, C₁-C₆ alkyl, C₂-C₆ alkenyl, C₂-C₆ alkynyl, C₃-C₈ cycloalkyl, C₁-C₆ alkoxy, C₁-C₆ haloalkyl, aryl, or heteroaryl; and
p is an integer from 0-4.
   In some embodiments, the compound is selected from the group consisting of:

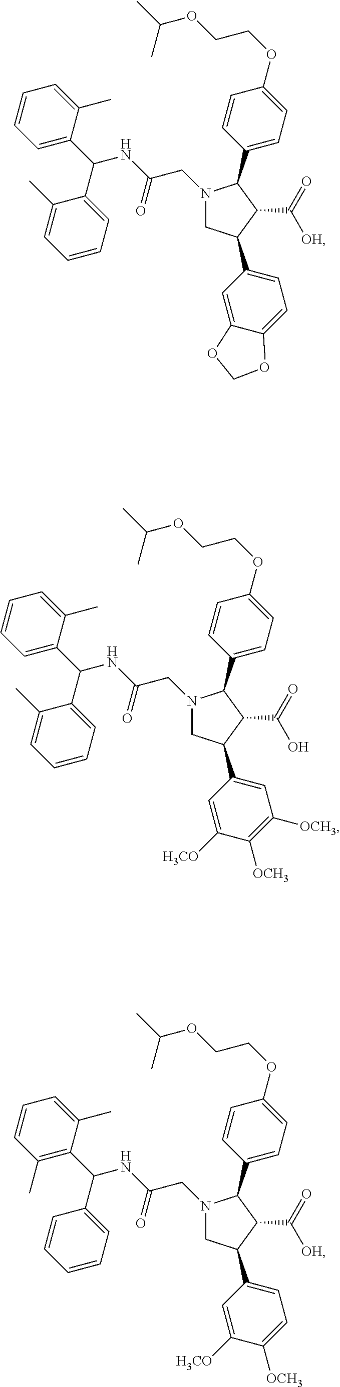 [SMILES: Cc1ccccc1C(NC(=O)CN1C[C@H](c2ccc3c(c2)OCO3)[C@@H](C(=O)O)[C@@H]1c1ccc(OCCOC(C)C)cc1)c1ccccc1C ; COc1cc([C@H]2CN(CC(=O)NC(c3ccccc3C)c3ccccc3C)[C@@H](c3ccc(OCCOC(C)C)cc3)[C@@H]2C(=O)O)cc(OC)c1OC ; COc1ccc([C@H]2CN(CC(=O)NC(c3ccccc3)c3c(C)cccc3C)[C@@H](c3ccc(OCCOC(C)C)cc3)[C@@H]2C(=O)O)cc1OC]

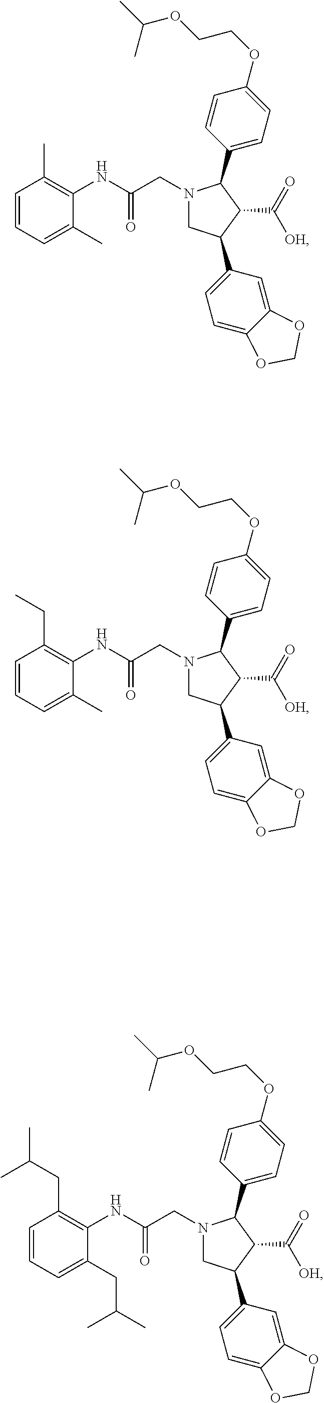 [SMILES: Cc1cccc(C)c1NC(=O)CN1C[C@H](c2ccc3c(c2)OCO3)[C@@H](C(=O)O)[C@@H]1c1ccc(OCCOC(C)C)cc1 ; CCc1cccc(C)c1NC(=O)CN1C[C@H](c2ccc3c(c2)OCO3)[C@@H](C(=O)O)[C@@H]1c1ccc(OCCOC(C)C)cc1 ; CC(C)Cc1cccc(CC(C)C)c1NC(=O)CN1C[C@H](c2ccc3c(c2)OCO3)[C@@H](C(=O)O)[C@@H]1c1ccc(OCCOC(C)C)cc1]

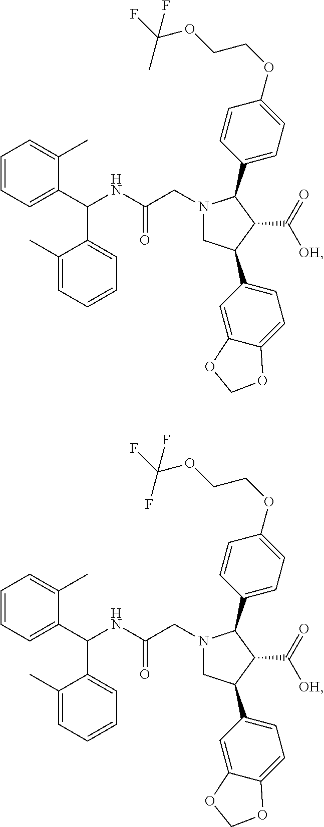 [SMILES: Cc1ccccc1C(NC(=O)CN1C[C@H](c2ccc3c(c2)OCO3)[C@@H](C(=O)O)[C@@H]1c1ccc(OCCOC(C)(F)F)cc1)c1ccccc1C ; Cc1ccccc1C(NC(=O)CN1C[C@H](c2ccc3c(c2)OCO3)[C@@H](C(=O)O)[C@@H]1c1ccc(OCCOC(F)(F)F)cc1)c1ccccc1C]

a stereoisomer thereof, a deuterated analog thereof, a fluorinated analog thereof, and a pharmaceutically acceptable salt or solvate thereof.

Also disclosed herein is a compound of Formula (7):

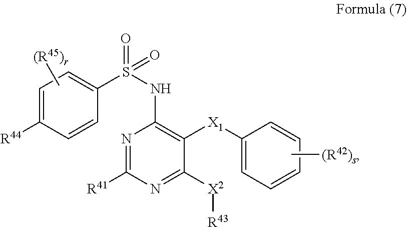

or a pharmaceutically acceptable salt or solvate thereof,
wherein,
R⁴¹ is hydrogen, halogen, —N(R⁴⁶)₂, —COOR⁴⁶, substituted or unsubstituted C₁-C₆ alkyl, substituted or unsubstituted C₃-C₈ cycloalkyl, substituted or unsubstituted C₂-C₇ heterocycloalkyl, substituted or unsubstituted aryl, or substituted or unsubstituted heteroaryl; wherein if R⁴¹ is substituted then itis substituted with 1, 2, or 3 substituents independently selected from fluoro, hydroxy, amino, —NH(C₁-C₄ alkyl), —N(C₁-C₄ alkyl)₂, nitro, cyano, C₁-C₄ alkyl, and C₁-C₄ alkoxy;
each X¹ and X² is independently —O—, —S—, —NR⁴⁶—, —CH₂—, or —(C═O)—;
each R⁴² and R⁴⁵ is independently deuterium, halogen, hydroxy, amino, nitro, cyano, C₁-C₆ alkyl, C₁-C₆ alkoxy, or C₁-C₆ haloalkyl;
R⁴⁴ is hydrogen, halogen, substituted or unsubstituted C₁-C₆ alkyl, substituted or unsubstituted C₁-C₆ fluoroalkyl, or substituted or unsubstituted C₁-C₆ alkoxy;
R⁴³ is substituted or unsubstituted C₁-C₆ alkyl, substituted or unsubstituted C₃-C₈ cycloalkyl, or substituted or unsubstituted C₂-C₇ heterocycloalkyl; wherein if R⁴³ is substituted then it is substituted with 1, 2, or 3 substituents independently selected from fluoro, hydroxy, amino, nitro, cyano, —N(R⁴⁶)₂, —COOR⁴⁶, —C(═O)R⁴⁶, —C(═O)NH(C₁-C₆ alkyl), —C(═O)NH(substituted or unsubstituted aryl), C₁-C₄ alkyl, C₁-C₄ alkoxy, C₂-C₆ alkenyl, C₂-C₆ alkynyl, substituted or unsubstituted C₃-C₈ cycloalkyl, substituted or unsubstituted C₂-C₇ heterocycloalkyl, substituted or unsubstituted aryl, and substituted or unsubstituted heteroaryl;
each R⁴⁶ is independently hydrogen or C₁-C₆ alkyl;
r is an integer from 0-4; and
s is an integer from 0-4.
   In some embodiments, the compound is selected from the group consisting of:

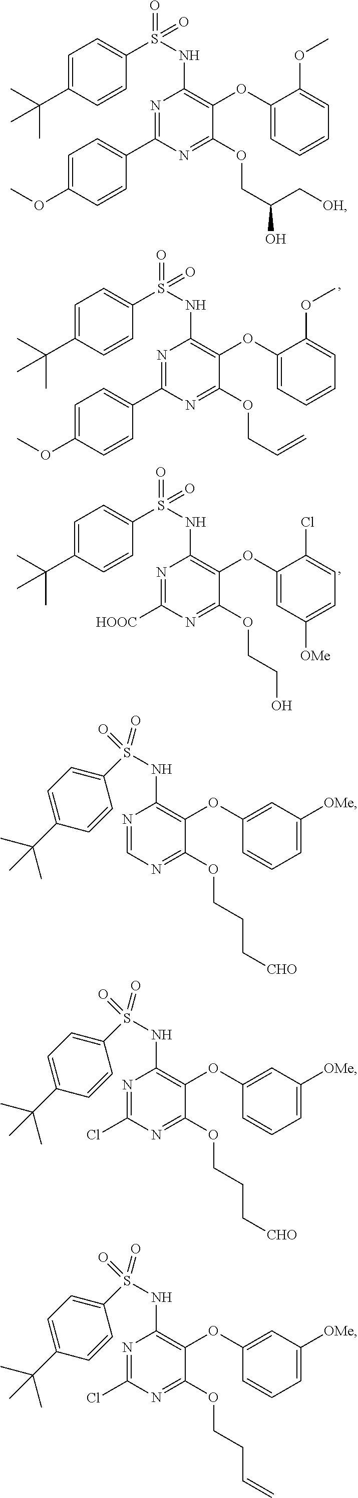

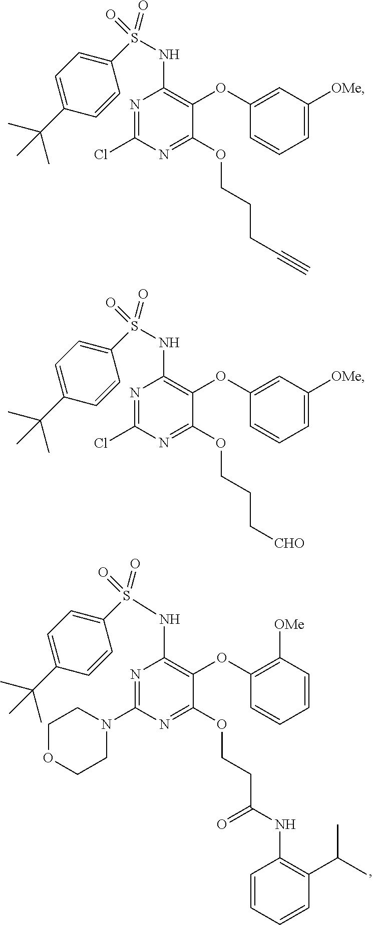 [SMILES: C#CCCCOc1nc(Cl)nc(NS(=O)(=O)c2ccc(C(C)(C)C)cc2)c1Oc1cccc(OC)c1 ; COc1cccc(Oc2c(NS(=O)(=O)c3ccc(C(C)(C)C)cc3)nc(Cl)nc2OCCCC=O)c1 ; COc1ccccc1Oc1c(NS(=O)(=O)c2ccc(C(C)(C)C)cc2)nc(N2CCOCC2)nc1OCCC(=O)Nc1ccccc1C(C)C]

a stereoisomer thereof, a deuterated analog thereof, a fluorinated analog thereof, and a pharmaceutically acceptable salt or solvate thereof.

Also disclosed herein is a compound of Formula (8):

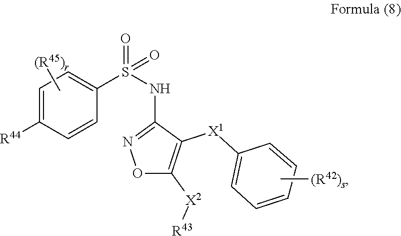

or a pharmaceutically acceptable salt or solvate thereof,
wherein,
each X¹ and X² is independently —O—, —S—, —NR⁴⁶—, —CH₂—, or —(C═O)—;
each R⁴² and R⁴⁵ is independently deuterium, halogen, hydroxy, amino, nitro, cyano, C₁-C₆ alkyl, C₁-C₆ alkoxy, or C₁-C₆ haloalkyl;
R⁴⁴ is hydrogen, halogen, substituted or unsubstituted C₁-C₆ alkyl, substituted or unsubstituted C₁-C₆ fluoroalkyl, or substituted or unsubstituted C₁-C₆ alkoxy;
R⁴³ is substituted or unsubstituted C₁-C₆ alkyl, substituted or unsubstituted C₃-C₈ cycloalkyl, or substituted or unsubstituted C₂-C₇ heterocycloalkyl; wherein if R⁴³ is substituted then it is substituted with 1, 2, or 3 substituents independently selected from fluoro, hydroxy, amino, nitro, cyano, —N(R⁴⁶)₂, —COOR⁴⁶, —C(═O)R⁴⁶, —C(═O)NH(C₁-C₆ alkyl), —C(═O)NH(substituted or unsubstituted aryl), C₁-C₄ alkyl, C₁-C₄ alkoxy, C₂-C₆ alkenyl, C₂-C₆ alkynyl, substituted or unsubstituted C₃-C₈ cycloalkyl, substituted or unsubstituted C₂-C₇ heterocycloalkyl, substituted or unsubstituted aryl, and substituted or unsubstituted heteroaryl;
each R⁴⁶ is independently hydrogen or C₁-C₆ alkyl;
r is an integer from 0-4; and
s is an integer from 0-4.
   In some embodiments, the compound is selected from the group consisting of:

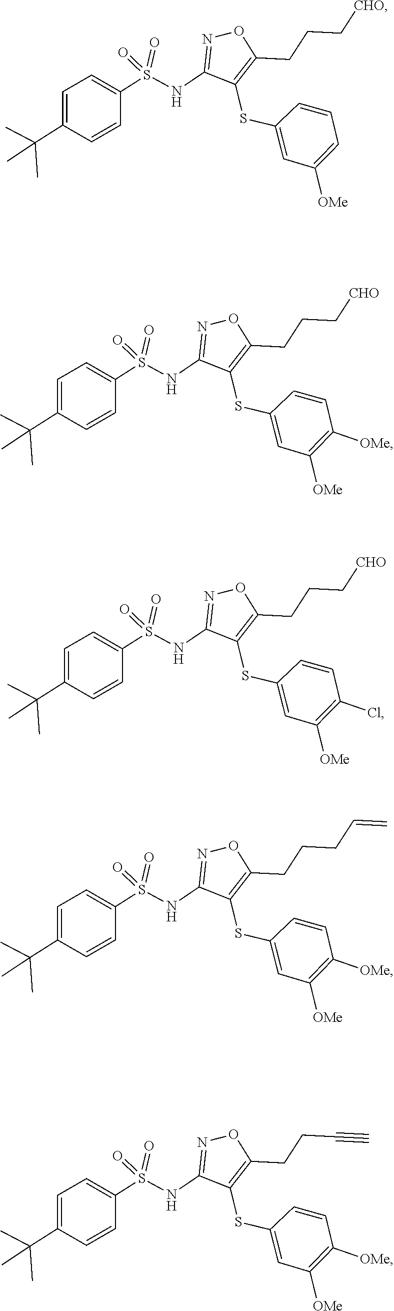

a stereoisomer thereof, a deuterated analog thereof, a fluorinated analog thereof, and a pharmaceutically acceptable salt or solvate thereof.

Also disclosed herein is a compound of Formula (9):

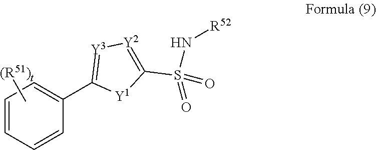

or a pharmaceutically acceptable salt or solvate thereof,
wherein,
each R⁵¹ is independently deuterium, halogen, hydroxy, nitro, cyano, —N(R⁵³)₂, —C(═O)R⁵³, —COOR⁵³, —C(═O)NHR⁵³, substituted or unsubstituted C₁-C₆ alkyl, substituted or unsubstituted C₁-C₆ alkoxy, substituted or unsubstituted C₁-C₆ haloalkyl, substituted or unsubstituted C₃-C₈ cycloalkyl, substituted or unsubstituted C₂-C₇ heterocycloalkyl, substituted or unsubstituted aryl, or substituted or unsubstituted heteroaryl; wherein if R⁵¹ is substituted then it is substituted with 1, 2, or 3 substituents independently selected from halogen, hydroxy, amino, —NH(C₁-C₄ alkyl), —N(C₁-C₄ alkyl)₂, nitro, cyano, C₁-C₄ alkyl, and C₁-C₄ alkoxy;
Y¹ is —O—, —S—, —NR⁵³—;
each Y² and Y³ is independently N or —CR⁵³—;
R⁵² is substituted or unsubstituted cycloalkyl, substituted or unsubstituted heterocycloalkyl, substituted or unsubstituted aryl, or substituted or unsubstituted heteroaryl; wherein if R⁵² is substituted then it is substituted with 1, 2, or 3 substituents independently selected from halogen, hydroxy, amino, —NH(C₁-C₄ alkyl), —N(C₁-C₄ alkyl)₂, nitro, cyano, C₁-C₄ alkyl, and C₁-C₄ alkoxy;
each R⁵³ is independently hydrogen, halogen, hydroxy, nitro, cyano, amino, C₁-C₆ alkyl, or C₁-C₄ alkoxy; and
t is an integer from 0-5.
   In some embodiments, the compound is selected from the group consisting of.

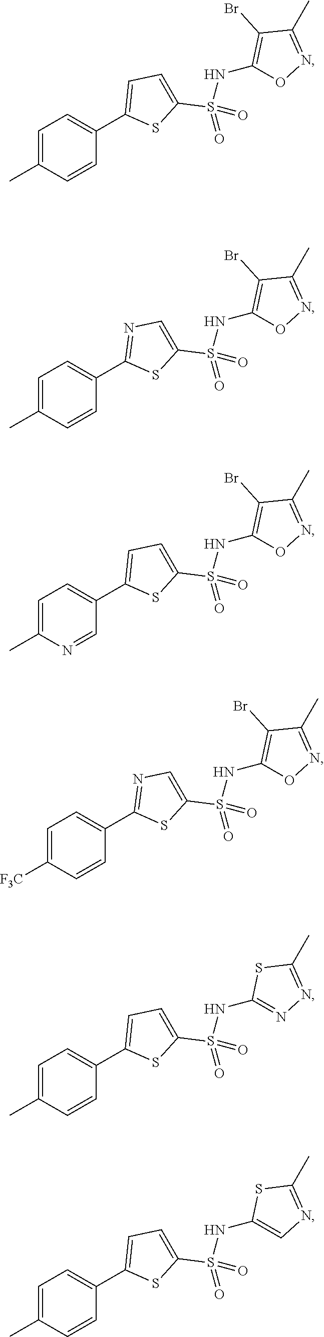

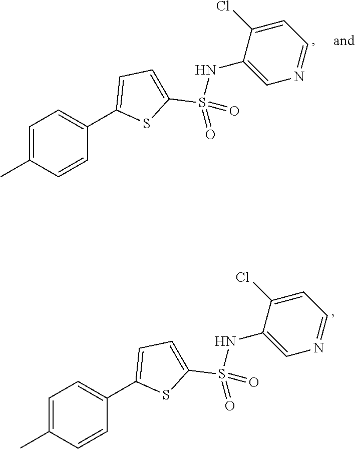 [SMILES: Cc1ccc(-c2ccc(S(=O)(=O)Nc3cnccc3Cl)s2)cc1 ; Cc1ccc(-c2ccc(S(=O)(=O)Nc3cnccc3Cl)s2)cc1]

a stereoisomer thereof, a deuterated analog thereof, a fluorinated analog thereof, and a pharmaceutically acceptable salt or solvate thereof.

Also disclosed herein are pharmaceutical compositions comprising a compound of formula (4), formula (5), formula (6), formula (7), formula (8) or formula (9) and a pharmaceutically acceptable excipient, diluent, or carrier. In some embodiments, the pharmaceutical composition comprises the pharmaceutically acceptable carrier, wherein the pharmaceutically acceptable carrier is dimethyl sulfoxide. In some embodiments, the compound is in a form of nanoparticles.

Also disclosed herein are methods of treating cancer in a subject in need thereof, comprising administering to the subject a pharmaceutical composition comprising a compound of formula (4), formula (5), formula (6), formula (7), formula (8) or formula (9), and a pharmaceutically acceptable excipient, diluent or carrier. In some embodiments, the method further comprises administering an immune checkpoint inhibitor. In some embodiments, the immune checkpoint inhibitor is an anti-PD1, or an anti-CTLA-4 agent for instance an antibody.

Also disclosed herein are methods of treating cancer in a subject in need thereof, comprising administering to the subject a compound of formula (4), formula (5), formula (6), formula (7), formula (8) or formula (9), wherein the compound is in an amount effective for treating or ameliorating at least one symptom of the cancer in the subject. In some embodiments, the method further comprises administering to the subject an additional anti-oncologic therapeutic agent, e.g., at least one immune checkpoint inhibitor. In some embodiments, the at least one immune checkpoint inhibitor comprises at least one anti-PD1 antibody, at least one anti-PD-L1 antibody, at least one anti-CTLA4 antibody, or any combination thereof. In some embodiments, the at least one anti-PD1 antibody comprises pidilizumab, BMS-936559, nivolumab, pembrolizumab, or any combination thereof. In some embodiments, the at least one anti-PD-L1 antibody comprises atezolizumab, avelumab, durvalumab, MDX-1105, or any combination thereof. In some embodiments, the compound and the additional anti-oncologic therapeutic agent are administered at different times. In some embodiments, the compound is administered 2, 3, 4, or 5 times of the frequency as the additional anti-oncologic therapeutic agent. In some embodiments, the compound is administered 3 times frequently as the additional anti-oncologic therapeutic agent. In some embodiments, the compound is administered 3 times every 2-3 weeks and the additional anti-oncologic therapeutic agent is administered 1 time every 2-3 weeks. In some embodiments, the compound is administered 3 times about every 21 days and the additional anti-oncologic therapeutic agent is administered 1 time about every 21 days. In some embodiments, the cancer is a solid tumor cancer, melanoma tumor, malignant melanoma, metastatic melanoma, malignant squamous cell carcinoma, metastatic squamous cell carcinoma, glioblastoma, brain cancer, pancreatic cancer, colon cancer, breast cancer, ovarian cancer, prostate cancer, or any combination thereof. In some embodiments, the subject is a human. In some embodiments, the subject is resistant to an immunotherapy before the treatment. In some embodiments, the administration restores Tumor Infiltrating Lymphocytes (TILs), intratumoral tertiary lymphoid organ (TLO) formation, or a combination thereof, in a tumor microenvironment.

Also disclosed herein is a method of forming a tertiary lymphoid organ (TLO) in a subject in need thereof, comprising administering to the subject a compound of formula (4), formula (5), formula (6), formula (7), formula (8) or formula (9), or a pharmaceutical composition comprising a compound of formula (4), formula (5), formula (6), formula (7), formula (8) or formula (9). In some embodiments, the method further comprises administering to the subject at least one additional therapeutic agent, for instance, an anti-oncologic therapeutic agent or an anti-microbial agent. In some embodiments, the at least one anti-oncologic agent comprises a bRAF inhibitor, an immune checkpoint inhibitor, a caspase-8 inhibitor, an ETAR antagonist, niacinamide, a chemotherapeutic agent, or any combination thereof. In some embodiments, the at least one anti-oncologic agent comprises at least one of the immune checkpoint inhibitor. In some embodiments, the at least one immune checkpoint inhibitor comprises at least one anti-PD1 antibody, at least one anti-PD-L1 antibody, at least one anti-CTLA4 antibody, or any combination thereof. In some embodiments, the at least one anti-PD1 antibody comprises pidilizumab, BMS-936559, nivolumab, pembrolizumab, or any combination thereof. In some embodiments, the at least one anti-PD-L1 antibody comprises atezolizumab, avelumab, durvalumab, MDX-1105, or any combination thereof. In some embodiments, the compound and the at least one additional anti-oncologic agent are administered at different times. In some embodiments, the compound is administered 2, 3, 4, or 5 times frequently as the immune checkpoint inhibitor. In some embodiments, the compound is administered 3 times frequently as the immune checkpoint inhibitor. In some embodiments, the compound is administered 3 times every 2-3 weeks and the immune checkpoint inhibitor is administered 1 time about every 2-3 weeks. In some embodiments, the compound is administered 3 times about every 21 days and the immune checkpoint inhibitor is administered 1 time about every 21 days. In some embodiments, the tumor is a solid tumor, melanoma tumor, solid tumor cancer, malignant melanoma, metastatic melanoma, malignant squamous cell carcinoma, metastatic squamous cell carcinoma, glioblastoma, brain cancer, pancreatic cancer, colon cancer, breast cancer, ovarian cancer, prostate cancer, or any combination thereof. In some embodiments, the subject is a human. In some embodiments, the subject is resistant to an immunotherapy before the treatment. In some embodiments, the ETBR antagonist is in a form of nanoparticles. In some embodiments, the ETBR antagonist is a non-deuterated BQ-788 analog.

Also disclosed herein are therapeutic compounds of formula (4), formula (5), formula (6), formula (7), formula (8) or formula (9), or pharmaceutically acceptable compositions comprising a compound of formula (4), formula (5), formula (6), formula (7), formula (8) or formula (9) for the treatment of solid tumors or cancer, e.g., ETBR-related cancers.

Also disclosed herein is an ETBR antagonist compound of formula (1):

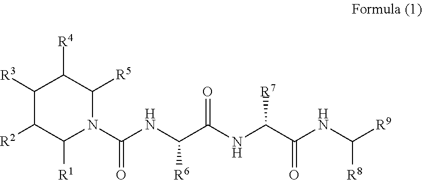

wherein
each of R¹-R⁵ is independently hydrogen, halogen, hydroxyl, deuterium, halogen, hydroxy, amino, nitro, optionally substituted C₁-C₈ alkyl, optionally substituted C₂-C₈ alkenyl, optionally substituted C₂-C₈ alkynyl, optionally substituted C₃-C₈-cycloalkyl, optionally substituted C₁-C₈ alkoxy, optionally substituted C₁-C₈ haloalkykl, optionally substituted aryl, or optionally substituted heteroaryl, e.g., an optionally substituted C₄-C₈ heteroaryl, or a deuterated form of the same, wherein one or more of the carbons in the piperidinyl group can be a heteroatom selected from O, N, or S, and/or wherein the ring may contain one or more double bonds, e.g., the group can be a pyridinyl, piperazinyl, pyridazinyl, pyrimidinyl, pyrazinyl, triazinyl, pyranyl, dioxanyl, thiazinyl, thianyl, thiopyranyl, dithianyl, trithianyl, morpholinyl, oxazinyl, or thiomorpholinyl group;
R⁶ is optionally substituted C₁-C₈ alkyl, optionally substituted C₂-C₈ alkenyl, optionally substituted C₂-C₈ alkynyl, optionally substituted C₃-C₈-cycloalkyl, optionally substituted C₁-C₈alkoxy, optionally substituted C₁-C₈ haloalkyl, optionally substituted aryl, or optionally substituted heteroaryl, wherein R⁶ optionally comprises deuterium or a group comprising deuterium;
R⁷ is optionally substituted cycloalkyl, optionally substituted heterocycloalkyl, optionally substituted aryl, optionally substituted heteroaryl, optionally substituted polycyclic ring system, optionally substituted bicyclic, optionally substituted heterobycyclic, e.g., an optionally substituted 9 or 10 membered bicyclic or heterobicyclic group, e.g., in indolinyl, imidazolyl, azaindolyl, benzofuranyl, indenyl, benzothiophenyl, purinyl, adeninyl, guaninyl, quinolinyl, quinolizinyl, phthalatyl, or phathalazinyl, wherein R7 optionally comprises deuterium;
R⁸ and R⁹ are independently optionally substituted C₁-C₈ alkyl, optionally substituted C₂-C₈ alkenyl, optionally substituted C₂-C₈ alkynyl, optionally substituted C₃-C₈-cycloalkyl, optionally substituted C₁-C₈ alkoxy, optionally substituted C₁-C₈ haloalkykl, optionally substituted aryl, optionally substituted heteroaryl, or —COOR′, or R⁸ and R⁹ may be taken together to form a optionally substituted cycloalkyl, optionally substituted cycloalkyl heterocycloalkyl, optionally substituted aryl, optionally substituted heteroaryl, or optionally substituted polycyclic ring system wherein R⁸ or R⁹ each optionally comprises deuterium; and
R′ is hydrogen, hydroxy, or C₁-C₈ alkyl;
or a stereoisomer or a pharmaceutically acceptable salt thereof.

Also disclosed herein is an ETBR antagonist compound of formula (2):

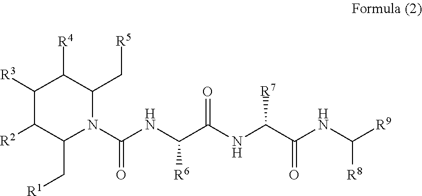

wherein
each of R¹-R⁵ is independently hydrogen, deuterium, halogen, hydroxy, amino, nitro, optionally substituted C₁-C₈ alkyl, optionally substituted C₂-C₈ alkenyl, optionally substituted C₂-C₈ alkynyl, optionally substituted C₃-C₈-cycloalkyl, optionally substituted C₁-C₈ alkoxy, optionally substituted C₁-C₈ haloalkykl, optionally substituted aryl, or optionally substituted heteroaryl;
R⁶ is optionally substituted C₁-C₈ alkyl, optionally substituted C₂-C₈ alkenyl, optionally substituted C₂-C₈ alkynyl, optionally substituted C₃-C₈-cycloalkyl, optionally substituted C₁-C₈ alkoxy, optionally substituted C₁-C₈ haloalkykl, optionally substituted aryl, or optionally substituted heteroaryl, wherein R6 optionally comprises deuterium;
R⁷ is optionally substituted cycloalkyl, optionally substituted heterocycloalkyl, optionally substituted aryl, optionally substituted heteroaryl, or a optionally substituted polycyclic ring system, wherein R⁷ optionally comprises deuterium;
R⁸ and R⁹ are independently optionally substituted C₁-C₈ alkyl, optionally substituted C₂-C₈ alkenyl, optionally substituted C₂-C₈ alkynyl, optionally substituted C₃-C₈-cycloalkyl, optionally substituted C₁-C₈ alkoxy, optionally substituted C₁-C₈ haloalkykl, optionally substituted aryl, optionally substituted heteroaryl, or —COOR′, or R⁸ and R⁹ may be taken together to form a substituted or unsubstituted cycloalkyl, substituted or unsubstituted cycloalkyl heterocycloalkyl, substituted or unsubstituted aryl, substituted or unsubstituted heteroaryl, or substituted or unsubstituted polycyclic ring system wherein R⁸ or R⁹ each optionally comprises deuterium; and
R′ is hydrogen, hydroxy, or C₁-C₈ alkyl;
or a stereoisomer or a pharmaceutically acceptable salt thereof.

Also disclosed herein is an ETBR antagonist compound of formula (3):

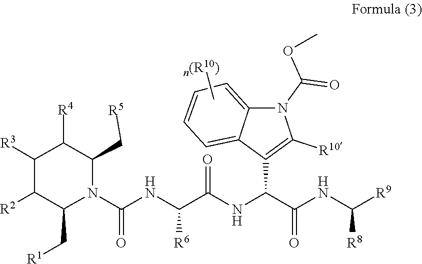

wherein
each of R¹-R⁵ is independently hydrogen, deuterium, halogen, hydroxy, amino, nitro, C₁-C₈ alkyl, C₂-C₈ alkenyl, C₂-C₈ alkynyl, C₃-C₈-cycloalkyl, C₁-C₈ alkoxy, C₁-C₈ haloalkykl, aryl, or heteroaryl;
R⁶ is C₁-C₈ alkyl, C₂-C₈ alkenyl, C₂-C₈ alkynyl, C₃-C₈-cycloalkyl, C₁-C₈ alkoxy, C₁-C₈ haloalkykl, aryl, or heteroaryl, wherein R6 optionally comprises deuterium;
R⁸ and R⁹ are independently optionally substituted C₁-C₈ alkyl, optionally substituted C2-C8 alkenyl, optionally substituted C₂-C₈ alkynyl, optionally substituted C₃-C₈-cycloalkyl, R⁸ and R⁹ are independently optionally substituted C₁-C₈ alkyl, optionally substituted C₂-C₈ alkenyl, optionally substituted C₂-C₈ alkynyl, optionally substituted C₃-C₈-cycloalkyl, optionally substituted C₁-C₈ alkoxy, optionally substituted C₁-C₈ haloalkykl, optionally substituted aryl, optionally substituted heteroaryl, or —COOR′, or R⁸ and R⁹ may be taken together to form a optionally substituted cycloalkyl, optionally substituted cycloalkyl, optionally substituted heterocycloalkyl, optionally substituted aryl, optionally substituted heteroaryl, or optionally substituted polycyclic ring system wherein R⁸ or R⁹ each optionally comprises deuterium; and
R¹⁰ and R^{10′} are independently hydrogen, deuterium, halogen, hydroxy, amino, nitro, optionally substituted C₁-C₈ alkyl, optionally substituted C₂-C₈ alkenyl, optionally substituted C₂-C₈ alkynyl, optionally substituted C₃-C₈-cycloalkyl, optionally substituted C₁-C₈ alkoxy, optionally substituted C₁-C₈ haloalkykl, optionally substituted aryl, or optionally substituted heteroaryl; and
n is an integer from 0-4;
a stereoisomer, or a pharmaceutically acceptable salt thereof.

In some embodiments, the optional substitution is independently a H, —OH, halogen, optionally substituted C₁-C₈ alkyl, optionally substituted C₁-C₈ alkoxy, optionally substituted C₂-C₈ alkenyl, optionally substituted C₂-C₈ alkynyl, optionally substituted C₃-C₈-cycloalkyl, optionally substituted C₁-C₈ alkoxy, optionally substituted C₁-C₈ haloalkykl, optionally substituted aryl, or optionally substituted heteroaryl, e.g., an optionally substituted C₄-C₈ heteroaryl, or a deuterated form of the same.

In some embodiments, R⁹ is —COOH. In some embodiments, R⁸ and R⁹ are taken together to form a tetrazolyl group.

### BRIEF DESCRIPTION OF THE DRAWINGS

FIG. 1 is an in vivo tumor growth curve over the time course of 21 days, which shows that a dual combination of an ETBR antagonist and an immune checkpoint inhibitor (anti-PD1 antibody) resulted in unexpected superior efficacy relative to the ETBR antagonist alone or the immune checkpoint inhibitor alone in a SM1 model.

FIG. 2 shows that tumor remnants after treatment of two ETBR antagonists respectively in combination with an immune checkpoint inhibitor had intratumoral TLOs (tertiary lymphoid organs). The first ETBR antagonist is deuterated BQ-788, dose 1 is 600 ng and dose 2 is 4 mg. The second ETBR antagonist is non-deuterated (nano-particle) BQ-788; dose 1 is 75 ng and dose 2 is 40 mg.

FIG. 3 is an in vivo tumor growth curve over the time course of 21 days, which shows that a dual combination of an ETBR antagonist and an immune checkpoint inhibitor (anti-PD1 antibody) results in unexpected superior efficacy relative to current standard drug combinations in a melanoma model. The ETBR antagonist induced intratumoral tertiary lymphoid organ (TLO) formation, and eradicated tumors. The syngeneic melanoma model V600E+ (BRAF mutated) SM1 tumor model was used in C57BL/6 mice to assess efficacy of the ETBR antagonist in combination with an immune checkpoint inhibitor as compared to a standard of treatment, dabrafenib with an immune checkpoint inhibitor. Dosing regime was 0.2 mg/kg 3× times per week IV.

FIG. 4 shows in high magnification that a dual combination of an ETBR antagonist (deuterated BQ-788) and an immune checkpoint inhibitor (an anti-PD1 antibody) eradicates melanoma tumors in 21 days, promotes robust CD8+ TIL infiltration and intratumoral tertiary lymphoid organ (TLO) formation. Histological examination of V600E+ melanoma tumor cells implanted into C57BL/6 mice 21 days after treatment of an ETBR antagonist and an immune checkpoint inhibitor.

FIG. 5 shows intratumoral TLO formation induced by combination therapy including an ETBR antagonist (deuterated BQ-788) and an immune checkpoint inhibitor (an anti-PD1 antibody). The staining of CD8+, CD4+ and Treg (FoxP3) lymphocytes indicates that the combination therapy promotes strong mobilization of lymphocytes to the tumor, which is associated with tumor eradication and positive patient outcomes. Histological examination of V600E+ melanoma tumor cells implanted into C57BL/6 mice 21 days after treatment with the combination therapy.

FIGS. 6A-D show peritumoral formation of TLOs following administration of an ETBR antagonist (deuterated BQ-788) monotherapy in a human melanoma SM1 mouse model.

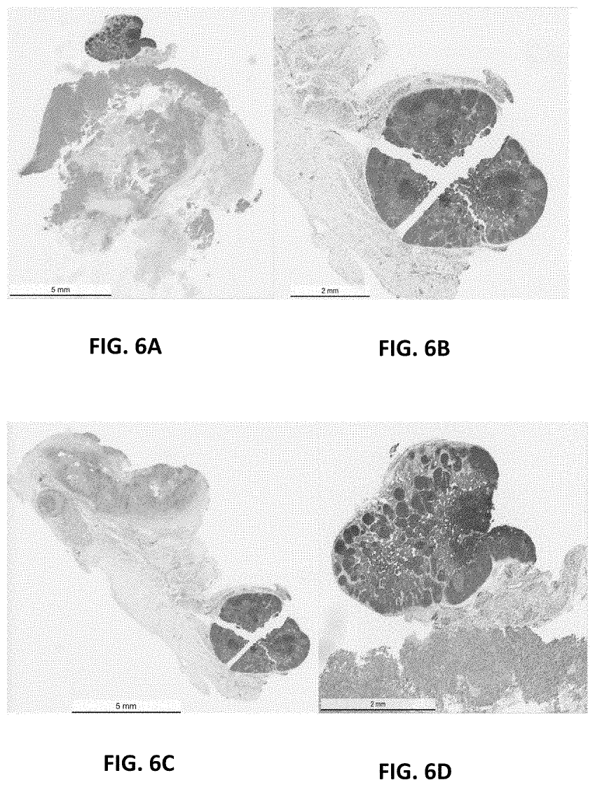

### DETAILED DESCRIPTION

Disclosed herein are ETBR antagonist compounds of formula (4), formula (5), formula (6), formula (7), formula (8) or formula (9), compositions comprising a compound of formula (4), formula (5), formula (6), formula (7), formula (8) or formula (9), and methods of using a compound of formula (4), formula (5), formula (6), formula (7), formula (8) or formula (9) for the treatment of cancer for example an ETBR-related cancer, e.g., malignant melanoma, metastatic melanoma, squamous cell carcinoma, glioblastoma, ovarian cancer, pancreatic cancer, or any combination thereof. ETBR antagonists of formula (4), formula (5), formula (6), formula (7), formula (8) or formula (9) are advantageous for treating ETBR-related cancers. The use of an ETBR antagonist of formula (4), formula (5), formula (6), formula (7), formula (8) or formula (9) improves biologic activity relative to the parent compound, as determined by measuring serum ET-1 levels, and results in at least one of increased stability, prolonged serum bioavailability, prolonged ETBR target engagement, or any combination thereof. In some embodiments, the subject treated is resistant to an immunotherapy. In some embodiments, the composition and method disclosed herein restores Tumor Infiltrating Lymphocytes (TILs) and/or intratumoral tertiary lymphoid organ (TLO) formation in a tumor microenvironment.

Also disclosed herein are methods of forming a tertiary lymphoid organ (TLO) in a subject in need thereof, comprising administering to the subject a TLO-forming compound disclosed herein. In some embodiments, the TLO-forming compound is an ETBR antagonist. In some embodiments, the ETBR antagonist is BQ-788, A192621, A-308165, IRL-1038, IRL-2500, RO-468443, BQ-017, or an analog thereof. In some embodiments, the ETBR antagonist is in a form of nanoparticles. In some embodiments, the ETBR antagonist is a non-deuterated BQ-788 analog. In some embodiments, the ETBR antagonist is not BQ-788. In some embodiments, the compound can be administered, e.g., at different times, with at least one additional anti-oncologic therapeutic agent such as an immune checkpoint inhibitor, e.g., anti-PD1 antibody or anti-PD-L1 antibody. In some embodiments, the compound can be in a pharmaceutically acceptable excipient that can comprise dimethyl sulfoxide (DMSO), LYOCELL (reversed cubic phase liquid crystal dispersion), soybean oil, INTRAVAIL (transmucosal absorption enhancement agents), PROTEK (protein stabilization excipients), or hydrogel, or any combination thereof. In some embodiments, tertiary lymphoid organs disclosed herein is formed within or adjacent to peripheral tissues, tumors, or cancers, or at or near sites of inflammation such as chronic inflammation, chronic infection, atherosclerosis, chronic kidney diseases, allograft rejection such as transplanted organs undergoing graft rejection, autoimmune diseases, pathologies, autoimmune diseases such as systemic lupus erythematosus and rheumatoid arthritis, or autoimmune-related diseases. In some embodiments, the TLO-forming compound is an endothelin A receptor (ETAR) antagonist, for example BQ123, BQ-610, A-127722, BSF-208075, BMS-182874, CI 1020, FR 139317, PD 151242, Sitaxsentan, and/or ZD4054. In some embodiments, tertiary lymphoid organ formation is not found in or after a tumor or cancer treatment. In some embodiments, tumor remnants in or after a cancer treatment do not form a tertiary lymphoid organ. In some embodiments, tertiary lymphoid organs form independently from a cancer treatment. In some embodiments, tertiary lymphoid organ formation accelerates or improves efficacy of a cancer treatment, e.g., reducing a tumor volume or eradicating a tumor, and shortening the treatment time.

Also disclosed herein are combinations that comprise at least one ETBR antagonist of formula (4), formula (5), formula (6), formula (7), formula (8) or formula (9), and at least one additional anti-oncologic therapeutic agent, administered either at the same time or at different times. In some embodiments, the at least one anti-oncologic agent can comprise a bRAF inhibitor, an immune checkpoint inhibitor, a caspase-8 inhibitor, an ETAR antagonist, niacinamide, a chemotherapeutic agent such as, e.g., a taxane, a kinase inhibitor, or other receptor antagonist or combination thereof. In some embodiments, the at least one anti-oncologic agent is an immune checkpoint inhibitor. In some embodiments, the immune checkpoint inhibitor is an anti-PD1 antibody or an anti-PD-L1 antibody. In some embodiments, the anti-PD1 antibody is nivolumab, pembrolizumab, pidilizumab, cemiplimab, or any combination thereof. In some embodiments, the anti-PD-L1 antibody is atezolizumab, MDX-1105, avelumab, durvalumab, or any combination thereof. In some embodiments, the ETRB antagonist and an anti-oncologic agent (i.e. immunocheckpoint inhibitors such as anti-anti-CTLA, anti-PDL1, and anti-PD1 antibodies) are administered at the same time (e.g. simultaneously. In some embodiments, the ETRB antagonist and the anti-oncologic agent (i.e. immunocheckpoint inhibitors such as anti-CTLA, anti-PDL1, and anti-PD1 antibodies) are administered at the different times (e.g. simultaneously. In some embodiments, the ETBR antagonist is administered once weekly, biweekly, monthly, or bimonthly. In some embodiments, the anti-oncologic agent (i.e. immunocheckpoint inhibitors such as anti-CTLA, anti-PD-L1, and anti-PD1 antibodies) is administered once weekly, biweekly, monthly, or bimonthly. In some embodiments, the ETBR antagonist is administered 2, 3, 4, or 5 times frequently as the additional anti-oncologic agent, for example that the ETBR antagonist is administered 3 times during 2-3 weeks (e.g., 21 days) while the additional anti-oncologic agent is administered 1 time during the 2-3 weeks (e.g., the 21 days). In some embodiments, the combination comprises an effective amount of the ETBR antagonist and an effective amount of an anti-oncologic agent. In some embodiments, the combination includes a pharmaceutically acceptable carrier for example dimethyl sulfoxide (DMSO), LYOCELL (reversed cubic phase liquid crystal dispersion), soybean oil, INTRAVAIL (transmucosal absorption enhancement agents), PROTEK (protein stabilization excipients), or hydrogel, or any combination thereof. In some embodiments, the combination is in separate unit dosage forms, for example, a first container that comprises the ETBR antagonist, and a second container that comprises the anti-oncologic agent. In some embodiments, the ETBR antagonist and/or the anti-oncolytic agent are in a controlled-release delivery system that comprises at least one of: (1) a biocompatible polymer, (2) a liposome preparation; (3) a DMSO solution, or a combination thereof. In some embodiments, the ETBR antagonist is in a form of nanoparticles. In some embodiments, the ETBR antagonist is a non-deuterated BQ-788 analog.

Also disclosed herein is a plurality of nanoparticles comprising an ETBR antagonist of formula (4), formula (5), formula (6), formula (7), formula (8) or formula (9). In some embodiments, a method for treating a cancer in a human subject in need thereof comprises administering the ETBR antagonist to the human subject the nanoparticles or a formulation thereof. In some embodiments, the ETBR antagonist in the nanoparticles is in an amount from about 0.01 μg to about 1 mg, for example from about 0.01 μg to about 0.1 μg. In some embodiments, the cancer is breast cancer, colon cancer, ovarian cancer, prostate cancer, melanoma, squamous cell carcinoma, glioblastoma, or any combination thereof. In some embodiments, the cancer is malignant melanoma or metastatic melanoma. In some embodiments, the cancer is ETBR-related metastatic brain cancer. In some embodiments, the ETBR-related metastatic brain cancer is metastatic melanoma-related brain cancer, metastatic squamous cell carcinoma-related brain cancer, glioblastoma, or any combination thereof. In some embodiments, the nanoparticles are administered with an additional anti-oncologic therapeutic agent, e.g., an immune checkpoint inhibitor. In some embodiments, the immune checkpoint inhibitor is administered at a same time as that of the ETBR antagonist. In some embodiments, the immune checkpoint inhibitor is administered at a time different from that of the ETBR antagonist. In some embodiments, the immune checkpoint inhibitor is an anti-PD1 antibody, e.g., nivolumab, pembrolizumab, pidilizumab, or any combination thereof. In some embodiments, the nanoparticles are administered with a cancer vaccine or a Chimeric Antigen Receptor T-Cell (CAR-T) therapy. In some embodiments, the nanoparticles are administered with a caspase-8 inhibitor.

### Definitions

Unless otherwise defined, all technical and scientific terms used herein have the same meaning as commonly understood by one of ordinary skill in the art to which this invention belongs. The terminology used in the description is for describing particular embodiments only and is not intended to be limiting of the invention.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the present disclosure.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

The term “combination therapy” refers to both concurrent administration (administration of two or more therapeutic agents at the same time) and time varied administration (administration of one or more therapeutic agents at a time different from that of the administration of an additional therapeutic agent or agents). In some embodiments, the therapeutic agents are present in the patient to some extent, for example at effective amounts, at the same time. In some embodiments, one or more of the compounds described herein, are administered in combination with at least one additional bioactive agent, especially including an anticancer agent.

The term “compound”, as used herein, unless otherwise indicated, refers to any specific chemical compound disclosed herein and includes tautomers, regioisomers, geometric isomers, and where applicable, stereoisomers, including optical isomers (enantiomers) and other stereoisomers (diastereomers) thereof, as well as pharmaceutically acceptable salts and derivatives (including prodrug forms) thereof where applicable, in context. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, in context to prodrug forms of compounds which have been modified to facilitate the administration and delivery of compounds to a site of activity. It is noted that in describing the present compounds, numerous substituents and variables associated with same, among others, are described. It is understood by those of ordinary skill that molecules which are described herein are stable compounds as generally described hereunder. When the bond is shown, both a double bond and single bond are represented within the context of the compound shown.

In some embodiments, the ETBR antagonist is BQ-788, or a pharmaceutically acceptable salt thereof. In some embodiments, BQ-788 is (2R)-2-[[(2R)-2-[[(2S)-2-[[(2R,6S)-2,6-dimethylpiperidine-1-carbonyl]amino]-4,4-dimethylpentanoyl]amino]-3-(1-methoxycarbonylindol-3-yl)propanoyl]amino]hexanoic acid.

In some embodiments, the ETBR antagonist is BQ-017, or a pharmaceutically acceptable salt thereof. In some embodiments, BQ-017 is (2R)-2-[[(2R)-3-(2-cyano-1H-indol-3-yl)-2-[[(2S)-2-[[(2R,6S)-2,6-dimethylpiperidine-1-carbonyl]amino]-3-methylbutanoyl]amino]propanoyl]amino]hexanoic acid.

In some embodiments, the ETBR antagonist is A192621, or a pharmaceutically acceptable salt thereof. In some embodiments, A192621 is (2R,3R,4S)-4-(1,3-benzodioxol-5-yl)-1-[2-(2,6-diethylanilino)-2-oxoethyl]-2-(4-propoxyphenyl)pyrrolidine-3-carboxylic acid.

In some embodiments, the ETBR antagonist is A-308165, or a pharmaceutically acceptable salt thereof. In some embodiments, A-308165 is (2R,3R,4S)-4-(1,3-benzodioxol-5-yl)-1-[2-[bis(2-methylphenyl)methylamino]-2-oxoethyl]-2-[4-(2-propan-2-yloxyethoxy)phenyl]pyrrolidine-3-carboxylic acid.

In some embodiments, the ETBR antagonist is IRL-1038, or a pharmaceutically acceptable salt thereof. In some embodiments, IRL-1038 is (3S)-3-[[(2S)-2-[[(2S)-2-[[(4R,7S,10S,13S,16R)-16-amino-7-benzyl-10-[(4-hydroxyphenyl)methyl]-6,9,12,15-tetraoxo-13-propan-2-yl-1,2-dithia-5,8,11,14-tetrazacycloheptadecane-4-carbonyl]amino]-3-(1H-imidazol-5-yl)propanoyl]amino]-4-methylpentanoyl]amino]-4-[[(2S,3S)-1-[[(2S,3S)-1-[[(1S)-1-carboxy-2-(1H-indol-3-yl)ethyl]amino]-3-methyl-1-oxopentan-2-yl]amino]-3-methyl-1-oxopentan-2-yl]amino]-4-oxobutanoic acid.

In some embodiments, the ETBR antagonist is IRL-2500, or a pharmaceutically acceptable salt thereof. In some embodiments, IRL-2500 is (2S)-2-[[(2R)-2-[(3,5-dimethylbenzoyl)-methylamino]-3-(4-phenylphenyl)propanoyl]amino]-3-(1H-indol-3-yl)propanoic acid.

In some embodiments, the ETBR antagonist is L017832, or a pharmaceutically acceptable salt thereof. In some embodiments, L017832 is 4-(tert-butyl)-N-(5-(3-methoxyphenoxy)-6-(4-oxobutoxy)pyrimidin-4-yl)benzenesulfonamide.

In some embodiments, the ETBR antagonist is RO-468443, or a pharmaceutically acceptable salt thereof. In some embodiments, RO-468443 is 4-tert-butyl-N-[6-[(2R)-2,3-dihydroxypropoxy]-5-(2-methoxyphenoxy)-2-(4-methoxyphenyl)pyrimidin-4-yl]benzenesulfonamide.

In some embodiments, the ETAR antagonist is BQ-610, or a pharmaceutically acceptable salt thereof. BQ-610 (2R)-2-[[(2R)-2-[[(2S)-2-(azepane-1-carbonylamino)-4-methylpentanoyl]amino]-3-(1-formylindol-3-yl)propanoyl]amino]-3-(1H-indol-3-yl)propanoic acid is a selective ETAR antagonist.

In some embodiments, the ETAR antagonist is A-127722, or a pharmaceutically acceptable salt thereof. A-127722 (2R,3R,4S)-4-(1,3-benzodioxol-5-yl)-1-[2-(dibutylamino)-2-oxoethyl]-2-(4-methoxyphenyl)pyrrolidine-3-carboxylic acid is a selective ETAR antagonist.

In some embodiments, the ETAR antagonist is BSF-208075, or a pharmaceutically acceptable salt thereof. BSF-208075 (2S)-2-(4,6-dimethylpyrimidin-2-yl)oxy-3-methoxy-3,3-diphenylpropanoic acid is a selective ETAR antagonist.

In some embodiments, the ETAR antagonist is BMS-182874, or a pharmaceutically acceptable salt thereof. BMS-182874 5-(dimethylamino)-N-(3,4-dimethyl-1,2-oxazol-5-yl)naphthalene-1-sulfonamide is a selective ETAR antagonist.

In some embodiments, the ETAR antagonist is CI 1020, or a pharmaceutically acceptable salt thereof. CI 1020 3-(1,3-benzodioxol-5-yl)-5-hydroxy-5-(4-methoxyphenyl)-4-[(3,4,5-trimethoxyphenyl)methyl]furan-2-one is a selective ETAR antagonist.

In some embodiments, the ETAR antagonist is FR 139317, or a pharmaceutically acceptable salt thereof. FR 139317 (2R)-2-[[(2R)-2-[[(2S)-2-(azepane-1-carbonylamino)-4-methylpentanoyl]amino]-3-(1-methylindol-3-yl)propanoyl]amino]-3-pyridin-2-ylpropanoic acid is a selective ETAR antagonist.

In some embodiments, the ETAR antagonist is PD 151242, or a pharmaceutically acceptable salt thereof. PD 151242 (2R)-2-[[(2R)-2-[[(2S)-2-(azepane-1-carbonylamino)-4-methylpentanoyl]amino]-3-(1-methylindol-3-yl)propanoyl]amino]-3-(4-hydroxyphenyl)propanoic acid is a selective ETAR antagonist.

In some embodiments, the ETAR antagonist is Sitaxsentan, or a pharmaceutically acceptable salt thereof. Sitaxsentan N-(4-chloro-3-methyl-1,2-oxazol-5-yl)-2-[2-(6-methyl-1,3-benzodioxol-5-yl)acetyl]thiophene-3-sulfonamide is a selective ETAR antagonist.

In some embodiments, the ETAR antagonist is ZD4054, or a pharmaceutically acceptable salt thereof. ZD4054 N-(3-methoxy-5-methylpyrazin-2-yl)-2-[4-(1,3,4-oxadiazol-2-yl)phenyl]pyridine-3-sulfonamide is a selective ETAR antagonist.

The term “alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (t-butyl), 3-methylhexyl, 2-methylhexyl, 1-ethyl-propyl, and the like. In some embodiments, the alkyl is methyl or ethyl. In some embodiments, the alkyl is —CH(CH₃)₂ or —C(CH₃)₃. Unless stated otherwise specifically in the specification, an alkyl group may be optionally substituted as described below. “Alkylene” or “alkylene chain” refers to a straight or branched divalent hydrocarbon chain linking the rest of the molecule to a radical group. In some embodiments, the alkylene is —CH₂—, —CH₂CH₂—, —CH₂CH(CH₃)CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—.

The term “alkoxy” refers to a radical of the formula —OR where R is an alkyl radical as defined. Unless stated otherwise specifically in the specification, an alkoxy group may be optionally substituted as described below. Representative alkoxy groups include, but are not limited to, methoxy, ethoxy, propoxy, butoxy, pentoxy. In some embodiments, the alkoxy is methoxy. In some embodiments, the alkoxy is ethoxy.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH.

The term “aryl” refers to an aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted. In some embodiments, an aryl group is partially reduced to form a cycloalkyl group defined herein. In some embodiments, an aryl group is fully reduced to form a cycloalkyl group defined herein.

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopropyl, cyclobutyl, cyclopentyl or cyclohexyl. In some embodiments, the monocyclic cycloalkyl is cyclopentenyl or cyclohexenyl. In some embodiments, the monocyclic cycloalkyl is cyclopentenyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted.

The term “fluoroalkyl” refers to an alkyl in which one or more hydrogen atoms are replaced by a fluorine atom. In one aspect, a fluoroalkyl is a C₁-C₆ fluoroalkyl. In some embodiments, a fluoroalkyl is selected from trifluoromethyl, difluoromethyl, fluoromethyl, 2,2,2-trifluoroethyl, 1-fluoromethyl-2-fluoroethyl, and the like.

The term “haloalkyl” denotes an alkyl group wherein at least one of the hydrogen atoms of the alkyl group has been replaced by same or different halogen atoms, particularly fluoro atoms. Examples of haloalkyl include monofluoro-, difluoro- or trifluoro-methyl, -ethyl or -propyl, for example 3,3,3-trifluoropropyl, 2-fluoroethyl, 2,2,2-trifluoroethyl, fluoromethyl, or trifluoromethyl. The term “perhaloalkyl” denotes an alkyl group where all hydrogen atoms of the alkyl group have been replaced by the same or different halogen atoms.

The term “heteroalkyl” refers to an alkyl group in which one or more skeletal atoms of the alkyl are selected from an atom other than carbon, e.g., oxygen, nitrogen (e.g. —NH—, —N(alkyl)-, or —N(aryl)-), sulfur (e.g. —S—, —S(═O)—, or —S(═O)₂—), or combinations thereof. In some embodiments, a heteroalkyl is attached to the rest of the molecule at a carbon atom of the heteroalkyl. In some embodiments, a heteroalkyl is attached to the rest of the molecule at a heteroatom of the heteroalkyl. In some embodiments, a heteroalkyl is a C₁-C₆ heteroalkyl. Representative heteroalkyl groups include, but are not limited to —OCH₂OMe, —OCH₂CH₂OH, —OCH₂CH₂OMe, or —OCH₂CH₂OCH₂CH₂NH₂.

The term “heterocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 12 carbons in the ring. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

The term “heteroaryl” refers to an aryl group that includes one or more ring heteroatoms selected from nitrogen, oxygen and sulfur. The heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1-4 N atoms in the ring. In some embodiments, a heteroaryl contains 4-6 N atoms in the ring. In some embodiments, a heteroaryl contains 0-4 N atoms, 0-1 O atoms, 0-1 P atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C₉ heteroaryl. In some embodiments, a heteroaryl group is partially reduced to form a heterocycloalkyl group defined herein. In some embodiments, a heteroaryl group is fully reduced to form a heterocycloalkyl group defined herein. Heteroaryl groups described herein that are substituted with a hydroxyl group may be present as tautomers. The heteroaryl groups described herein encompass all tautomers including non-aromatic tautomers.

The term “optionally substituted” or “substituted” means that the referenced group is optionally substituted with one or more additional group(s) individually and independently selected from D, halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —CO₂(C₁-C₄ alkyl), —C(═O)NH₂, —C(═O)NH(C₁-C₄ alkyl), —C(═O)N(C₁-C₄ alkyl)₂, —S(═O)₂NH₂, —S(═O)₂NH(C₁-C₄ alkyl), —S(═O)₂N(C₁-C₄ alkyl)₂, C₁-C₄ alkyl, C₃-C₆ cycloalkyl, C₁-C₄ fluoroalkyl, C₁-C₄ heteroalkyl, C₁-C₄ alkoxy, C₁-C₄ fluoroalkoxy, —SC₁-C₄ alkyl, —S(═O)C₁-C₄ alkyl, and —S(═O)₂(C₁-C₄ alkyl). In some embodiments, optional substituents are independently selected from D, halogen, —CN, —NH₂, —OH, —NH(CH₃), —N(CH₃)₂, —NH(cyclopropyl), —CH₃, —CH₂CH₃, —CF₃, —OCH₃, and —OCF3. In some embodiments, substituted groups are substituted with one or two of the preceding groups. In some embodiments, an optional substituent on an aliphatic carbon atom (acyclic or cyclic) includes oxo (═O).

The term “tautomer” refers to a proton shift from one atom of a molecule to another atom of the same molecule. The compounds presented herein may exist as tautomers. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH. Some examples of tautomeric interconversions include:

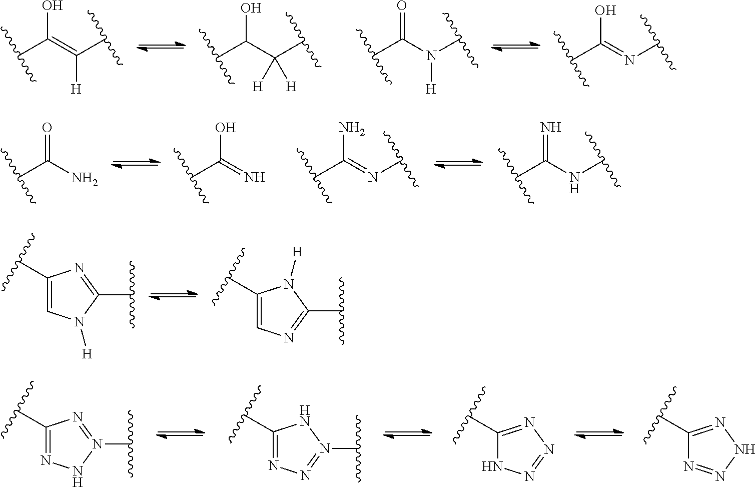

The term “anti-oncologic agent” is used to describe an anti-cancer agent, which may be combined with compounds according to the present disclosure to treat cancer. These agents include, for example, everolimus, niacinamide, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitor, an AKT inhibitor, an mTORC1/2 inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR1 KRX-0402, lucanthone, LY317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258); 3-[5-(methylsulfonylpiperadinemethyl)-indolyl-quinolone, vatalanib, AG-013736, AVE-0005, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, adriamycin, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gleevec, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonist, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

The term “pharmaceutically acceptable salt” is used throughout the specification to describe, where applicable, a salt form of one or more of the compounds described herein which are presented to increase the solubility of the compound in the gastric juices of the patient's gastrointestinal tract in order to promote dissolution and the bioavailability of the compounds. Pharmaceutically acceptable salts include those derived from pharmaceutically acceptable inorganic or organic bases and acids, where applicable. Suitable salts include those derived from alkali metals such as potassium and sodium, alkaline earth metals such as calcium, magnesium and ammonium salts. In some embodiments, sodium and potassium salts are suitable neutralization salts of the phosphates.

The term “pharmaceutically acceptable derivative” is used throughout the specification to describe any pharmaceutically acceptable prodrug form (such as an ester, amide other prodrug group), which, upon administration to a patient, provides directly or indirectly the present compound or an active metabolite of the present compound.

The term “effective” is used to describe an amount of a compound, composition or component which, when used within the context of its intended use, effects an intended result. The term “effective” subsumes all other effective amount or effective concentration terms, which are otherwise described or used in the present application.

The term “therapeutically effective amount” refers to that amount which is sufficient to effect treatment, as defined herein, when administered to a mammal in need of such treatment.

The term “patient” or “subject” is used throughout the specification to describe an animal, for example a human, or a domesticated animal, to whom treatment, including prophylactic treatment, with the compositions according to the present disclosure is provided. Treatment does not require the supervision of a medical professional and may be done by the subject apart from a medical professional. For treatment of those infections, conditions or disease states which are specific for a specific animal such as a human patient, the term patient refers to that specific animal, including a domesticated animal such as a dog or cat or a farm animal such as a horse, cow, sheep, etc. In general, in the present disclosure, the term patient refers to a human patient unless otherwise stated or implied from the context of the use of the term. Activation of the ETBR by endothelins such as ET-1 and ET-3, results in a variety of molecular events that promote melanoma invasion and metastasis. Without being bound by any particular theory, it is hypothesized that while the majority of melanomas express ETBR, a subset of these also expresses the ETBR activator ET-1 and/or ET-3. It is this subset that is therefore most likely dependent upon ETBR activation for viability, invasive potential and metastatic potential. Thus, this subset of patients is most likely to respond to ETBR blockade. Furthermore, this subset of patients is least likely to response to immune based therapy.

TLOs

Disclosed herein are methods of forming a tertiary lymphoid organ (TLO) in a subject in need thereof, comprising administering to the subject a TLO-forming compound. Tertiary lymphoid organs are accumulations of lymphocytes and stromal cells in an organized structure that occur outside of secondary lymphoid organs (SLOs). The tertiary lymphoid organs disclosed herein are formed within (intratumoral) or adjacent (peritumoral) to tumors, or cancers, or at or near sites of inflammation such as chronic inflammation, chronic infection, atherosclerosis, chronic kidney diseases, allograft rejection such as transplanted organs undergoing graft rejection, autoimmune diseases, pathologies, autoimmune diseases such as systemic lupus erythematosus and rheumatoid arthritis, or autoimmune-related diseases. In some embodiments, the TLO is intratumoral. In some embodiments, the TLO is peritumoral. In some embodiments, tertiary lymphoid organ formation accelerates or improves efficacy of a cancer treatment, e.g., reducing a tumor volume or eradicating a tumor, and shortening the treatment time.

In some embodiments, the TLO-forming compound is an ETBR antagonist. In some embodiments, the ETBR antagonist is BQ-788, A192621, A-308165, IRL-1038, IRL-2500, RO-468443, BQ-017, or an analog thereof. In some embodiments, the ETBR antagonist is a compound of Formula (1), Formula (2), Formula (3), Formula (4), Formula (5), Formula (6), Formula (7), Formula (8) or Formula (9). In some embodiments, the ETBR antagonist is a non-deuterated BQ-788 analog. In some embodiments, the ETBR antagonist is not BQ-788.

In some embodiments, the TLO-forming compound is an endothelin A receptor (ETAR) antagonist. In some embodiments, the compound is BQ123, BQ-610, A-127722, BSF-208075, BMS-182874, CI 1020, FR 139317, PD 151242, Sitaxsentan, and/or ZD4054.

In some embodiments, the TLO-forming compound is in a form of nanoparticles. In some embodiments, the TLO-forming compound is in a pharmaceutically acceptable excipient that comprises dimethyl sulfoxide (DMSO), LYOCELL (reversed cubic phase liquid crystal dispersion), soybean oil, INTRAVAIL (transmucosal absorption enhancement agents), PROTEK (protein stabilization excipients), or hydrogel, or any combination thereof. In some embodiments, the TLO-forming compound is BQ-788 and is in the form of nano-particles.

In some embodiments, the ETBR antagonist is administered by IV. In some embodiments, a composition comprising BQ-788 is administered by IV.

In some embodiments, the ETBR antagonist is administered at a low dose. In some embodiments, the ETBR antagonist is administered at a dose of about 50 ug/day to about 500 ug/day, about 50 ug/day to about 400 ug/day, about 50 ug/day to about 300 ug/day, about 50 ug/day to about 200 ug/day, about 100 ug/day to about 150 ug/day. In some embodiments, the ETBR antagonist is administered 3 days per week (i.e., 1 cycle). In some embodiments, the ETBR antagonist is administered for 6 cycles.

In some embodiments, the TLO-forming compound is administered with at least one additional anti-oncologic therapeutic agent. In some embodiments, the additional anti-oncolytic agent is an immune checkpoint inhibitor. In some embodiments, the immune checkpoint inhibitor is an anti-PD1 antibody. In some embodiments, the immune checkpoint inhibitor is an anti-PD-L1 antibody.

In some embodiments, tertiary lymphoid organ formation is not found in the subject after completion of treatment.

In some embodiments, the method comprises administering BQ-788 and an anti-PD-1 antibody. In some embodiments, the BQ-788 is administered as nanoparticles. In some embodiments, BQ-788 is not deuterated. In some embodiments, BQ-788 is administered as an IV formulation. In some embodiments, the BQ-788 is administered at a dose of between 50 ug and 200 ug/day for 3 days in a week (i.e., 1 cycle). In some embodiments, administration of a cycle of BQ-788 is repeated 1 time, 2 times, 3 times, 4 times, or 5 times.

Compounds

Disclosed herein is an ETBR antagonist, e.g., an analog of BQ-788 as described herein. In some embodiments, the description provides a composition comprising at least one ETBR antagonist, e.g., an analog of BQ-788 as described herein, and a pharmaceutically acceptable carrier. In some embodiments, the description provides a composition, e.g., a pharmaceutical composition, comprising an effective amount of at least one ETBR antagonist, e.g., an analog of BQ-788 as described herein, and a pharmaceutically acceptable carrier. In some embodiments, the pharmaceutical composition as described herein can be in unit dosage form configured for administration one or more times, for example, one or more times per day, per week, or per month. In some embodiments, the ETBR antagonist is not BQ-788. In some embodiments, the ETBR antagonist is a non-deuterated BQ-788 analog.

In some embodiments, a compound disclosed herein is of Formula (1):

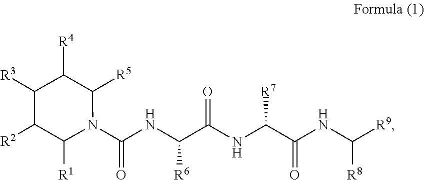

a stereoisomer thereof, a deuterated analog, a fluorinated analog, or a pharmaceutically acceptable salt thereof,
wherein:
each of R¹, R², R³, R⁴, or R⁵ is independently hydrogen, halogen, hydroxyl, deuterium, halogen, hydroxy, amino, nitro, optionally substituted C₁-C₈ alkyl, optionally substituted C₂-C₈ alkenyl, optionally substituted C₂-C₈ alkynyl, optionally substituted C₃-C₈ cycloalkyl, optionally substituted C₁-C₈ alkoxy, optionally substituted C₁-C₈ haloalkykl, optionally substituted aryl, or optionally substituted heteroaryl, optionally wherein one or more of the carbons in the piperidinyl ring can be a heteroatom selected from O, N, or S, or wherein the piperidinyl ring may contain one or more double bonds;
R⁶ is optionally substituted C₁-C₈ alkyl, optionally substituted C₂-C₈ alkenyl, optionally substituted C₂-C₈ alkynyl, optionally substituted C₃-C₈-cycloalkyl, optionally substituted C₁-C₈ alkoxy, optionally substituted C₁-C₈ haloalkykl, optionally substituted aryl, or optionally substituted heteroaryl, wherein R⁶ optionally comprises deuterium;
R⁷ is optionally substituted cycloalkyl, optionally substituted heterocycloalkyl, optionally substituted aryl, optionally substituted heteroaryl, optionally substituted polycyclic ring system, optionally substituted bicyclic, optionally substituted heterobicyclic, wherein R⁷ optionally comprises deuterium;
R⁸ and R⁹ are independently optionally substituted C₁-C₈ alkyl, optionally substituted C₂-C₈ alkenyl, optionally substituted C₂-C₈ alkynyl, optionally substituted C₃-C₈ cycloalkyl, optionally substituted C₁-C₈ alkoxy, optionally substituted C₁-C₈ haloalkyl, optionally substituted aryl, optionally substituted heteroaryl, or —COOR′, or R⁸ and R⁹ may be taken together to form a optionally substituted cycloalkyl, optionally substituted cycloalkyl heterocycloalkyl, optionally substituted aryl, optionally substituted heteroaryl, or optionally substituted polycyclic ring system, wherein R⁸ or R⁹ each optionally comprises deuterium; and
R′ is hydrogen, hydroxy, or C₁-C₈ alkyl.

In some embodiments, a compound disclosed herein is of Formula (2):

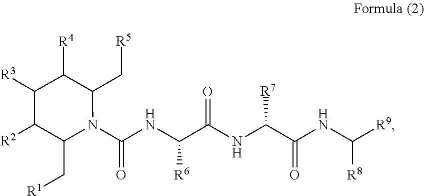

a stereoisomer thereof, a deuterated analog, a fluorinated analog, or a pharmaceutically acceptable salt thereof,
wherein:
each of R¹, R², R³, R⁴, or R⁵ is independently hydrogen, deuterium, halogen, hydroxy, amino, nitro, C₁-C₈ alkyl, C₂-C₈ alkenyl, C₂-C₈ alkynyl, C₃-C₈ cycloalkyl, C₁-C₈ alkoxy, C₁-C₈ haloalkykl, aryl, or heteroaryl;
R⁶ is C₁-C₈ alkyl, C₂-C₈ alkenyl, C₂-C₈ alkynyl, C₃-C₈ cycloalkyl, C₁-C₈ alkoxy, C₁-C₈ haloalkykl, aryl, or heteroaryl, wherein R⁶ optionally comprises deuterium;
R⁷ is substituted or unsubstituted cycloalkyl, substituted or unsubstituted heterocycloalkyl, substituted or unsubstituted aryl, substituted or unsubstituted heteroaryl, or a substituted or unsubstituted polycyclic ring system, wherein R⁷ optionally comprises deuterium;
R⁸ and R⁹ are independently C₁-C₈ alkyl, C₂-C₈ alkenyl, C₂-C₈ alkynyl, C₃-C₈-cycloalkyl, C₁-C₈ alkoxy, C₁-C₈ haloalkykl, aryl, heteroaryl, or —COOR′, or R⁸ and R⁹ may be taken together to form a substituted or unsubstituted cycloalkyl, substituted or unsubstituted cycloalkyl heterocycloalkyl, substituted or unsubstituted aryl, substituted or unsubstituted heteroaryl, or substituted or unsubstituted polycyclic ring system, wherein R⁸ or R⁹ each optionally comprises deuterium; and
R′ is hydrogen, hydroxy, or C₁-C₈ alkyl.

In some embodiments, a compound disclosed herein is of Formula (3):

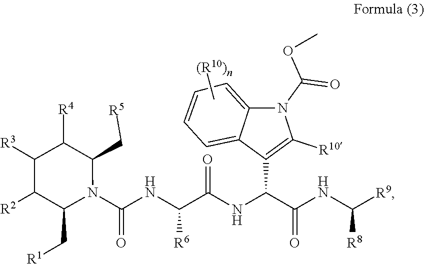

a stereoisomer thereof, a deuterated analog, a fluorinated analog, or a pharmaceutically acceptable salt thereof,
wherein:
each of R¹R², R³, R⁴, or R⁵ is independently hydrogen, deuterium, halogen, hydroxy, amino, nitro, C₁-C₈ alkyl, C₂-C₈ alkenyl, C₂-C₈ alkynyl, C₃-C₈ cycloalkyl, C₁-C₈ alkoxy, C₁-C₈ haloalkyl, aryl, or heteroaryl;
R⁶ is C₁-C₈ alkyl, C₂-C₈ alkenyl, C₂-C₈ alkynyl, C₃-C₈-cycloalkyl, C₁-C₈ alkoxy, C₁-C₈ haloalkykl, aryl, or heteroaryl, wherein R⁶ optionally comprises deuterium;
R⁸ and R⁹ are independently C₁-C₈ alkyl, C₂-C₈ alkenyl, C₂-C₈ alkynyl, C₃-C₈ cycloalkyl, R⁸ and R⁹ are independently C₁-C₈ alkyl, C₂-C₈ alkenyl, C₂-C₈ alkynyl, C₃-C₈-cycloalkyl, C₁-C₈ alkoxy, C₁-C₈ haloalkykl, aryl, heteroaryl, or —COOR′, or R⁸ and R⁹ may be taken together to form a substituted or unsubstituted cycloalkyl, substituted or unsubstituted cycloalkyl heterocycloalkyl, substituted or unsubstituted aryl, substituted or unsubstituted heteroaryl, or substituted or unsubstituted polycyclic ring system, wherein R⁸ or R⁹ each optionally comprises deuterium;
R¹⁰ and R^{10′} are independently hydrogen, deuterium, halogen, hydroxy, amino, nitro, C₁-C₈ alkyl, C₂-C₈ alkenyl, C₂-C₈ alkynyl, C₃-C₈-cycloalkyl, C₁-C₈ alkoxy, C₁-C₈ haloalkykl, aryl, or heteroaryl; and
n is an integer from 0-4.

In some embodiments, disclosed herein is a compound of Formula (4):

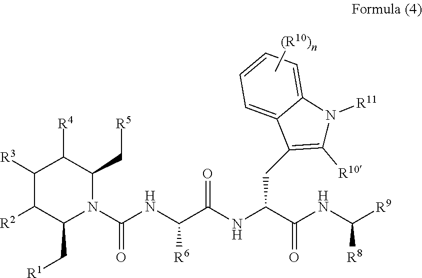

a stereoisomer thereof, a deuterated analog, a fluorinated analog, or a pharmaceutically acceptable salt thereof,
wherein,
each of R¹-R⁵ is independently hydrogen, deuterium, halogen, hydroxy, amino, nitro, cyano, C₁-C₈ alkyl, C₂-C₈ alkenyl, C₂-C₈ alkynyl, C₃-C₈ cycloalkyl, C₁-C₈ alkoxy, C₁-C₈ haloalkyl, aryl, or heteroaryl;
R⁶ is C₁-C₈ alkyl, C₂-C₈ alkenyl, C₂-C₈ alkynyl, C₃-C₈ cycloalkyl, C₁-C₈ alkoxy, C₁-C₈ haloalkyl, aryl, or heteroaryl, wherein R⁶ optionally comprises deuterium;
R⁸ and R⁹ are each independently C₁-C₈ alkyl, C₂-C₈ alkenyl, C₂-C₈ alkynyl, C₃-C₈ cycloalkyl, C₁-C₈ alkoxy, C₁-C₈ haloalkyl, aryl, heteroaryl, or —COOR′, or R⁸ and R⁹ may be taken together to form substituted or unsubstituted C₃-C₈ cycloalkyl, substituted or unsubstituted C₂-C₇ heterocycloalkyl, substituted or unsubstituted aryl, substituted or unsubstituted heteroaryl, or substituted or unsubstituted polycyclic ring system, wherein R⁸ or R⁹ each optionally comprises deuterium;
R¹⁰ and R^{10′} are each independently hydrogen, deuterium, halogen, hydroxy, amino, nitro, cyano, C₁-C₈ alkyl, C₂-C₈ alkenyl, C₂-C₈ alkynyl, C₃-C₈ cycloalkyl, C₁-C₈ alkoxy, C₁-C₈ haloalkyl, aryl, or heteroaryl;
R¹¹ is hydrogen, deuterium, C₁-C₈ alkyl, C₁-C₈ haloalkyl, C₃-C₈ cycloalkyl, aryl, heteroaryl, or —COOR′;
each R′ is independently hydrogen or C₁-C₈ alkyl; and
n is an integer from 0-4.

In some embodiments, disclosed herein is a compound of Formula (4a):

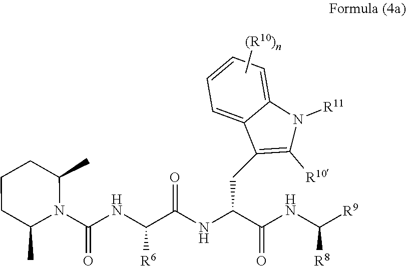

a stereoisomer thereof, a deuterated analog, a fluorinated analog, or a pharmaceutically acceptable salt thereof,
wherein
R⁶ is C₁-C₈ alkyl, C₂-C₈ alkenyl, C₂-C₈ alkynyl, C₃-C₈ cycloalkyl, C₁-C₈ alkoxy, C₁-C₈ haloalkyl, aryl, or heteroaryl, wherein R⁶ optionally comprises deuterium;
R⁸ and R⁹ are each independently C₁-C₈ alkyl, C₂-C₈ alkenyl, C₂-C₈ alkynyl, C₃-C₈ cycloalkyl, C₁-C₈ alkoxy, C₁-C₈ haloalkyl, aryl, heteroaryl, or —COOR′, or R⁸ and R⁹ may be taken together to form substituted or unsubstituted C₃-C₈ cycloalkyl, substituted or unsubstituted C₂-C₇ heterocycloalkyl, substituted or unsubstituted aryl, substituted or unsubstituted heteroaryl, or substituted or unsubstituted polycyclic ring system wherein R⁸ or R⁹ each optionally comprises deuterium;
R¹⁰ and R^{10′} are each independently hydrogen, deuterium, halogen, hydroxy, amino, nitro, cyano, C₁-C₈ alkyl, C₂-C₈ alkenyl, C₂-C₈ alkynyl, C₃-C₈ cycloalkyl, C₁-C₈ alkoxy, C₁-C₈ haloalkyl, aryl, or heteroaryl;
R¹¹ is hydrogen, deuterium, C₁-C₈ alkyl, C₁-C₈ haloalkyl, C₃-C₈ cycloalkyl, aryl, heteroaryl, or —COOR′;
each R′ is independently hydrogen or C₁-C₈ alkyl; and
n is an integer from 0-4.

In some embodiments, disclosed herein is a compound of Formula (4b):

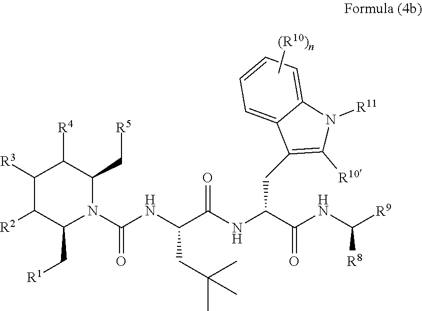

a stereoisomer thereof, a deuterated analog, a fluorinated analog, or a pharmaceutically acceptable salt thereof,
wherein
each of R¹-R⁵ is independently hydrogen, deuterium, halogen, hydroxy, amino, nitro, cyano, C₁-C₈ alkyl, C₂-C₈ alkenyl, C₂-C₈ alkynyl, C₃-C₈ cycloalkyl, C₁-C₈ alkoxy, C₁-C₈ haloalkyl, aryl, or heteroaryl;
R⁸ and R⁹ are each independently C₁-C₈ alkyl, C₂-C₈ alkenyl, C₂-C₈ alkynyl, C₃-C₈ cycloalkyl, C₁-C₈ alkoxy, C₁-C₈ haloalkyl, aryl, heteroaryl, or —COOR′, or R⁸ and R⁹ may be taken together to form substituted or unsubstituted C₃-C₈ cycloalkyl, substituted or unsubstituted C₂-C₇ heterocycloalkyl, substituted or unsubstituted aryl, substituted or unsubstituted heteroaryl, or substituted or unsubstituted polycyclic ring system, wherein R⁸ or R⁹ each optionally comprises deuterium;
R¹⁰ and R^{10′} are each independently hydrogen, deuterium, halogen, hydroxy, amino, nitro, cyano, C₁-C₈ alkyl, C₂-C₈ alkenyl, C₂-C₈ alkynyl, C₃-C₈ cycloalkyl, C₁-C₈ alkoxy, C₁-C₈ haloalkyl, aryl, or heteroaryl;
R¹¹ is hydrogen, deuterium, C₁-C₈ alkyl, C₁-C₈ haloalkyl, C₃-C₈ cycloalkyl, aryl, heteroaryl, or —COOR′;
each R′ is independently hydrogen or C₁-C₈ alkyl; and
n is an integer from 0-4.

In some embodiments, disclosed herein is a compound of Formula (4c):

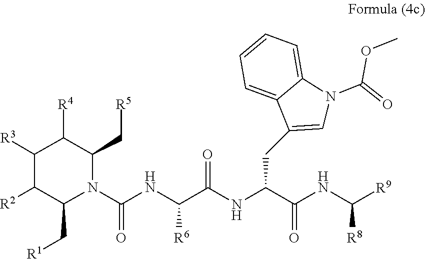

a stereoisomer thereof, a deuterated analog, a fluorinated analog, or a pharmaceutically acceptable salt thereof,
wherein
each of R¹-R⁵ is independently hydrogen, deuterium, halogen, hydroxy, amino, nitro, cyano, C₁-C₈ alkyl, C₂-C₈ alkenyl, C₂-C₈ alkynyl, C₃-C₈ cycloalkyl, C₁-C₈ alkoxy, C₁-C₈ haloalkyl, aryl, or heteroaryl;
R⁶ is C₁-C₈ alkyl, C₂-C₈ alkenyl, C₂-C₈ alkynyl, C₃-C₈ cycloalkyl, C₁-C₈ alkoxy, C₁-C₈ haloalkyl, aryl, or heteroaryl, wherein R⁶ optionally comprises deuterium;
R⁸ and R⁹ are each independently C₁-C₈ alkyl, C₂-C₈ alkenyl, C₂-C₈ alkynyl, C₃-C₈ cycloalkyl, C₁-C₈ alkoxy, C₁-C₈ haloalkyl, aryl, heteroaryl, or —COOR′, or R⁸ and R⁹ may be taken together to form substituted or unsubstituted C₃-C₈ cycloalkyl, substituted or unsubstituted C₂-C₇ heterocycloalkyl, substituted or unsubstituted aryl, substituted or unsubstituted heteroaryl, or substituted or unsubstituted polycyclic ring system, wherein R⁸ or R⁹ each optionally comprises deuterium; and
R′ is hydrogen or C₁-C₈ alkyl.

In some embodiments, disclosed herein is a compound of Formula (4d):

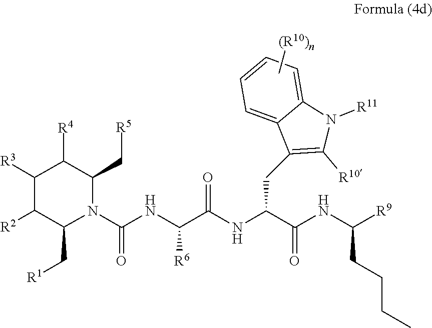

a stereoisomer thereof, a deuterated analog, a fluorinated analog, or a pharmaceutically acceptable salt thereof,
wherein
each of R¹-R⁵ is independently hydrogen, deuterium, halogen, hydroxy, amino, nitro, cyano, C₁-C₈ alkyl, C₂-C₈ alkenyl, C₂-C₈ alkynyl, C₃-C₈ cycloalkyl, C₁-C₈ alkoxy, C₁-C₈ haloalkyl, aryl, or heteroaryl;
R⁶ is C₁-C₈ alkyl, C₂-C₈ alkenyl, C₂-C₈ alkynyl, C₃-C₈ cycloalkyl, C₁-C₈ alkoxy, C₁-C₈ haloalkyl, aryl, or heteroaryl, wherein R⁶ optionally comprises deuterium;
R⁹ is C₁-C₈ alkyl, C₂-C₈ alkenyl, C₂-C₈ alkynyl, C₃-C₈ cycloalkyl, C₁-C₈ alkoxy, C₁-C₈ haloalkyl, aryl, heteroaryl, or —COOR′, wherein R⁹ each optionally comprises deuterium;
each R¹⁰ and R^{10′} is independently hydrogen, deuterium, halogen, hydroxy, amino, nitro, cyano, C₁-C₈ alkyl, C₂-C₈ alkenyl, C₂-C₈ alkynyl, C₃-C₈ cycloalkyl, C₁-C₈ alkoxy, C₁-C₈ haloalkyl, aryl, or heteroaryl;
R¹¹ is hydrogen, deuterium, C₁-C₈ alkyl, C₁-C₈ haloalkyl, C₃-C₈ cycloalkyl, aryl, heteroaryl, or —COOR′;
each R′ is independently hydrogen or C₁-C₈ alkyl; and
n is an integer from 0-4.

In some embodiments, disclosed herein is a compound, or a pharmaceutically acceptable salt or solvate thereof, selected from:

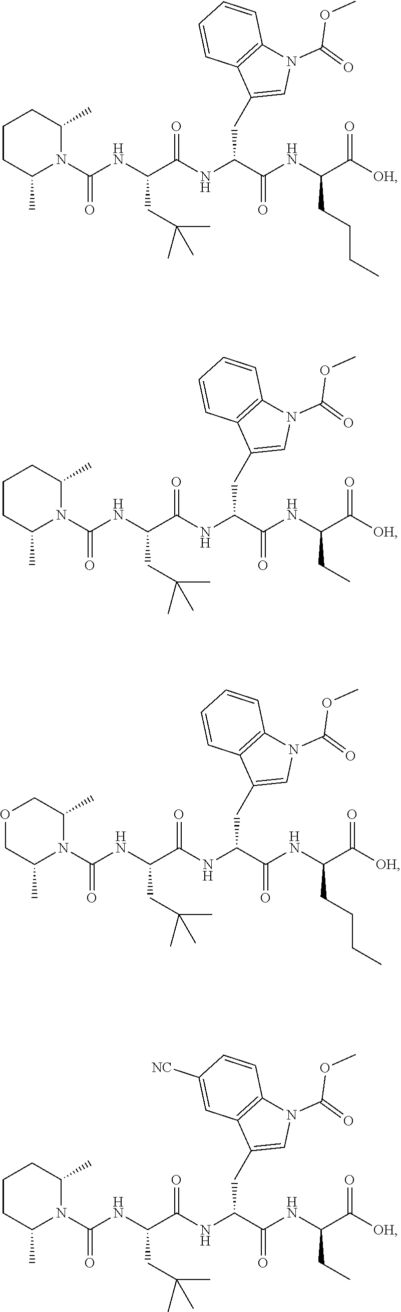 [SMILES: CCCC[C@@H](NC(=O)[C@@H](Cc1cn(C(=O)OC)c2ccccc12)NC(=O)[C@H](CC(C)(C)C)NC(=O)N1[C@H](C)CCC[C@@H]1C)C(=O)O ; CC[C@@H](NC(=O)[C@@H](Cc1cn(C(=O)OC)c2ccccc12)NC(=O)[C@H](CC(C)(C)C)NC(=O)N1[C@H](C)CCC[C@@H]1C)C(=O)O ; CCCC[C@@H](NC(=O)[C@@H](Cc1cn(C(=O)OC)c2ccccc12)NC(=O)[C@H](CC(C)(C)C)NC(=O)N1[C@H](C)COC[C@@H]1C)C(=O)O ; CC[C@@H](NC(=O)[C@@H](Cc1cn(C(=O)OC)c2ccc(C#N)cc12)NC(=O)[C@H](CC(C)(C)C)NC(=O)N1[C@H](C)CCC[C@@H]1C)C(=O)O]

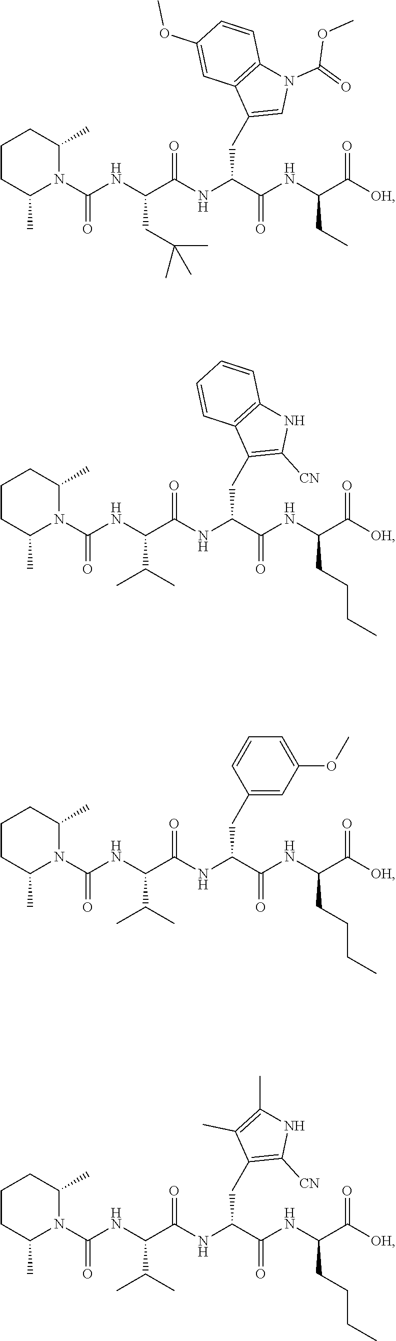 [SMILES: CC[C@@H](NC(=O)[C@@H](Cc1cn(C(=O)OC)c2ccc(OC)cc12)NC(=O)[C@H](CC(C)(C)C)NC(=O)N1[C@H](C)CCC[C@@H]1C)C(=O)O ; CCCC[C@@H](NC(=O)[C@@H](Cc1c(C#N)[nH]c2ccccc12)NC(=O)[C@@H](NC(=O)N1[C@H](C)CCC[C@@H]1C)C(C)C)C(=O)O ; CCCC[C@@H](NC(=O)[C@@H](Cc1cccc(OC)c1)NC(=O)[C@@H](NC(=O)N1[C@H](C)CCC[C@@H]1C)C(C)C)C(=O)O ; CCCC[C@@H](NC(=O)[C@@H](Cc1c(C#N)[nH]c(C)c1C)NC(=O)[C@@H](NC(=O)N1[C@H](C)CCC[C@@H]1C)C(C)C)C(=O)O]

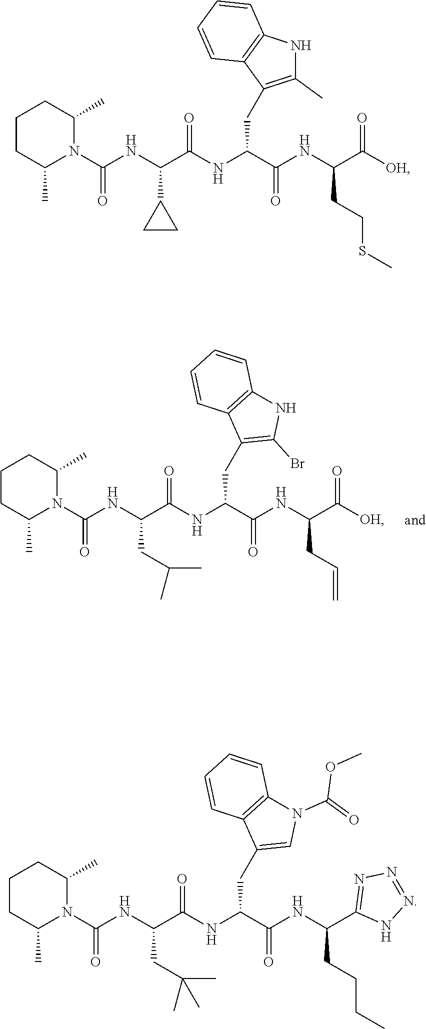 [SMILES: CSCC[C@@H](NC(=O)[C@@H](Cc1c(C)[nH]c2ccccc12)NC(=O)[C@@H](NC(=O)N1[C@H](C)CCC[C@@H]1C)C1CC1)C(=O)O ; C=CC[C@@H](NC(=O)[C@@H](Cc1c(Br)[nH]c2ccccc12)NC(=O)[C@H](CC(C)C)NC(=O)N1[C@H](C)CCC[C@@H]1C)C(=O)O ; "and" ; CCCC[C@@H](NC(=O)[C@@H](Cc1cn(C(=O)OC)c2ccccc12)NC(=O)[C@H](CC(C)(C)C)NC(=O)N1[C@H](C)CCC[C@@H]1C)c1nnn[nH]1]

In some embodiments, disclosed herein is a compound of Formula (5), or a pharmaceutically acceptable salt or solvate thereof:

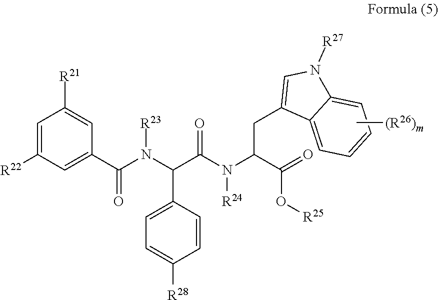

wherein,
each R²¹ and R²² is independently hydrogen, deuterium, halogen, hydroxy, amino, nitro, cyano, C₁-C₆ alkyl, C₃-C₈ cycloalkyl, or C₁-C₆ alkoxy;
each R²³ and R²⁴ is independently hydrogen or C₁-C₄ alkyl;
R²⁵ is hydrogen or C₁-C₆ alkyl;
each R²⁶ is independently deuterium, halogen, hydroxy, amino, nitro, cyano, C₁-C₆ alkyl, C₂-C₆ alkenyl, C₂-C₆ alkynyl, C₃-C₈ cycloalkyl, C₁-C₆ alkoxy, C₁-C₆ haloalkyl, aryl, or heteroaryl;
R²⁷ is hydrogen, deuterium, C₁-C₆ alkyl, C₁-C₆ haloalkyl, C₃-C₈ cycloalkyl, aryl, heteroaryl, or —COOR²⁹;
R²⁸ is substituted or unsubstituted C₃-C₈ cycloalkyl, substituted or unsubstituted C₂-C₇ heterocycloalkyl, substituted or unsubstituted aryl, or substituted or unsubstituted heteroaryl;
R²⁹ is hydrogen or C₁-C₆ alkyl; and
m is an integer from 0-4.

In some embodiments, disclosed herein is a compound, or a pharmaceutically acceptable salt or solvate thereof, selected from:

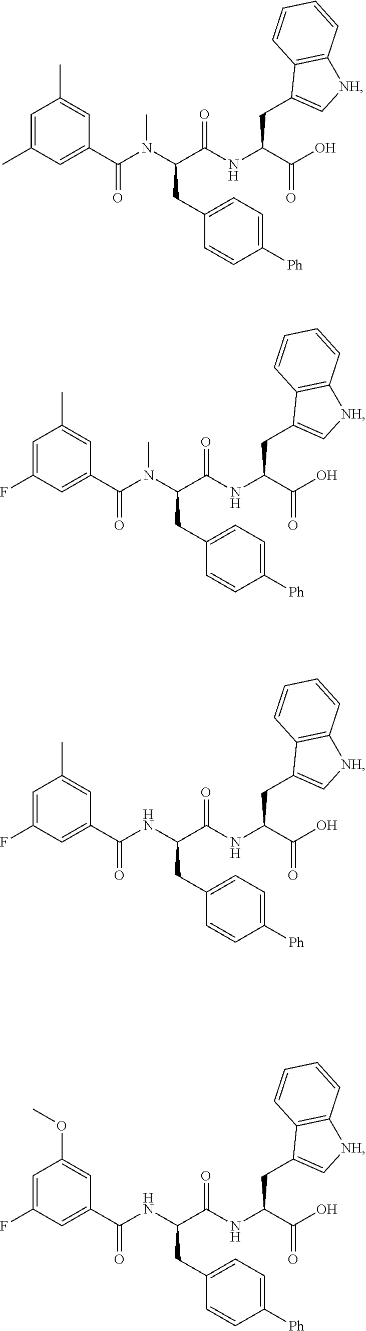 [SMILES: Cc1cc(C)cc(C(=O)N(C)[C@H](Cc2ccc(-c3ccccc3)cc2)C(=O)N[C@@H](Cc2c[nH]c3ccccc23)C(=O)O)c1 ; Cc1cc(F)cc(C(=O)N(C)[C@H](Cc2ccc(-c3ccccc3)cc2)C(=O)N[C@@H](Cc2c[nH]c3ccccc23)C(=O)O)c1 ; Cc1cc(F)cc(C(=O)N[C@H](Cc2ccc(-c3ccccc3)cc2)C(=O)N[C@@H](Cc2c[nH]c3ccccc23)C(=O)O)c1 ; COc1cc(F)cc(C(=O)N[C@H](Cc2ccc(-c3ccccc3)cc2)C(=O)N[C@@H](Cc2c[nH]c3ccccc23)C(=O)O)c1]

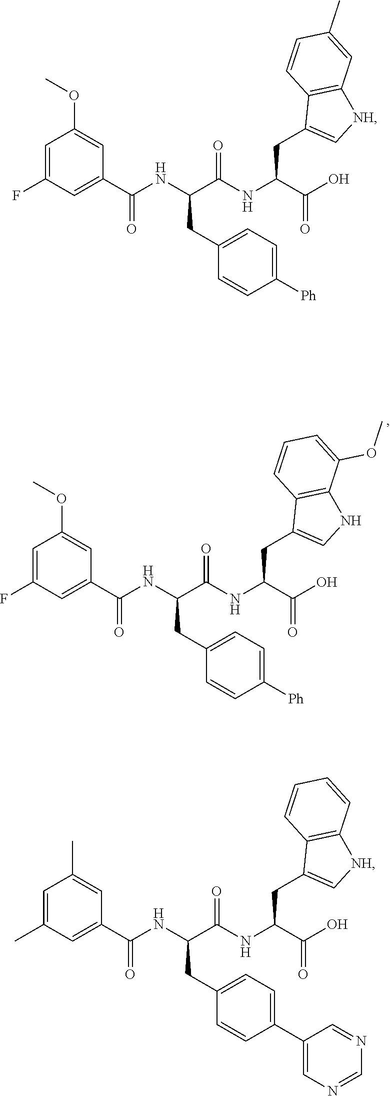

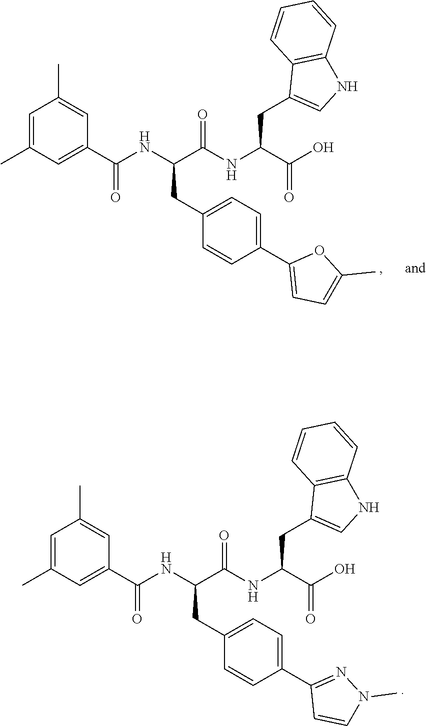

In some embodiments, disclosed herein is a compound of Formula (6), or a pharmaceutically acceptable salt or solvate thereof:

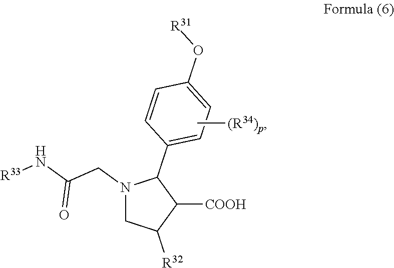

or a pharmaceutically acceptable salt or solvate thereof,
wherein,
R³¹ is substituted or unsubstituted C₁-C₆ alkyl, substituted or unsubstituted C₃-C₈ cycloalkyl, or substituted or unsubstituted C₂-C₇ heterocycloalkyl; wherein if R³¹ is substituted then it is substituted with 1, 2, or 3 substituents independently selected from fluoro, hydroxy, amino, —NH(C₁-C₄ alkyl), —N(C₁-C₄ alkyl)₂, nitro, cyano, C₁-C₄ alkyl, and C₁-C₄ alkoxy;
R³² is substituted or unsubstituted cycloalkyl, substituted or unsubstituted heterocycloalkyl, substituted or unsubstituted aryl, or substituted or unsubstituted heteroaryl;
R³³ is substituted or unsubstituted cycloalkyl, substituted or unsubstituted heterocycloalkyl, substituted or unsubstituted aryl, substituted or unsubstituted heteroaryl, or —CH(CR³⁵)₂, wherein each R³⁵ is independently substituted or unsubstituted C₁-C₆ alkyl, substituted or unsubstituted C₃-C₈ cycloalkyl, substituted or unsubstituted C₂-C₇ heterocycloalkyl, substituted or unsubstituted aryl, or substituted or unsubstituted heteroaryl; wherein if R³³ or R³⁵ is substituted then it is substituted with 1, 2, or 3 substituents independently selected from fluoro, hydroxy, amino, —NH(C₁-C₄ alkyl), —N(C₁-C₄ alkyl)₂, nitro, cyano, C₁-C₄ alkyl, and C₁-C₄ alkoxy;
each R³⁴ is independently deuterium, halogen, hydroxy, amino, nitro, cyano, C₁-C₆ alkyl, C₂-C₆ alkenyl, C₂-C₆ alkynyl, C₃-C₈ cycloalkyl, C₁-C₆ alkoxy, C₁-C₆ haloalkyl, aryl, or heteroaryl; and
p is an integer from 0-4.

In some embodiments, disclosed herein is a compound, or a pharmaceutically acceptable salt or solvate thereof, selected from:

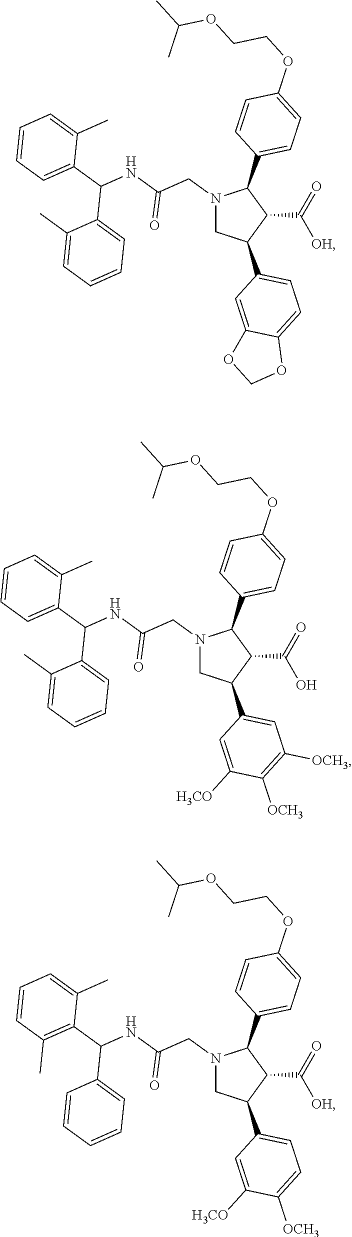 [SMILES: Cc1ccccc1C(NC(=O)CN1C[C@H](c2ccc3c(c2)OCO3)[C@@H](C(=O)O)[C@@H]1c1ccc(OCCOC(C)C)cc1)c1ccccc1C ; COc1cc([C@H]2CN(CC(=O)NC(c3ccccc3C)c3ccccc3C)[C@@H](c3ccc(OCCOC(C)C)cc3)[C@@H]2C(=O)O)cc(OC)c1OC ; COc1ccc([C@H]2CN(CC(=O)NC(c3ccccc3)c3c(C)cccc3C)[C@@H](c3ccc(OCCOC(C)C)cc3)[C@@H]2C(=O)O)cc1OC]

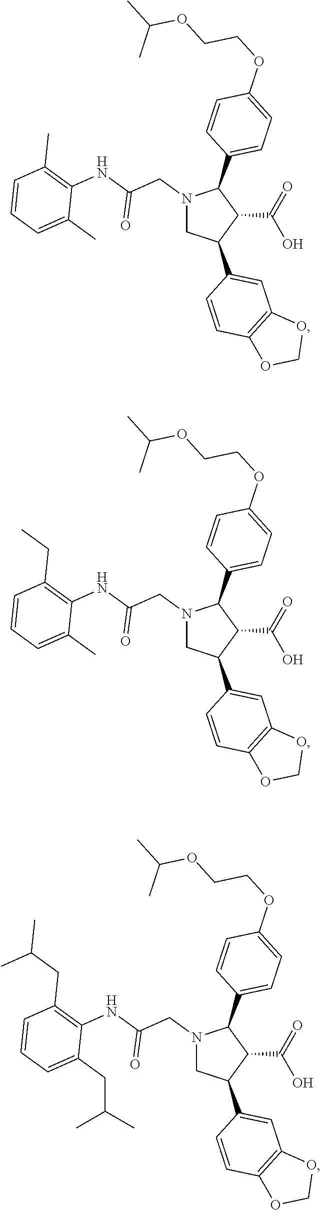 [SMILES: Cc1cccc(C)c1NC(=O)CN1C[C@H](c2ccc3c(c2)OCO3)[C@@H](C(=O)O)[C@@H]1c1ccc(OCCOC(C)C)cc1 ; CCc1cccc(C)c1NC(=O)CN1C[C@H](c2ccc3c(c2)OCO3)[C@@H](C(=O)O)[C@@H]1c1ccc(OCCOC(C)C)cc1 ; CC(C)Cc1cccc(CC(C)C)c1NC(=O)CN1C[C@H](c2ccc3c(c2)OCO3)[C@@H](C(=O)O)[C@@H]1c1ccc(OCCOC(C)C)cc1]

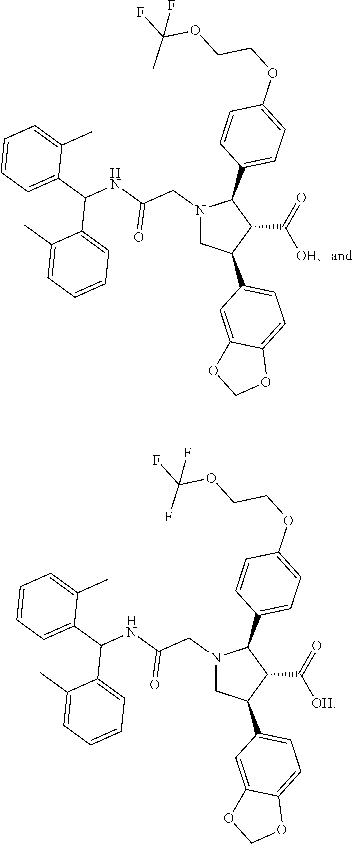 [SMILES: Cc1ccccc1C(NC(=O)CN1C[C@H](c2ccc3c(c2)OCO3)[C@@H](C(=O)O)[C@@H]1c1ccc(OCCOC(C)(F)F)cc1)c1ccccc1C ; "and" ; Cc1ccccc1C(NC(=O)CN1C[C@H](c2ccc3c(c2)OCO3)[C@@H](C(=O)O)[C@@H]1c1ccc(OCCOC(F)(F)F)cc1)c1ccccc1C]

In some embodiments, disclosed herein is a compound of Formula (7), or a pharmaceutically acceptable salt or solvate thereof:

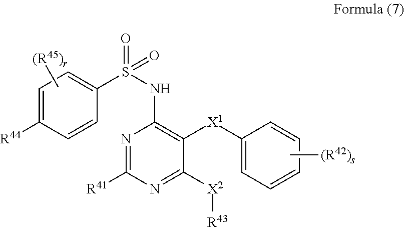

wherein,
R⁴¹ is hydrogen, halogen, —N(R⁴⁶)₂, —COOR⁴⁶, substituted or unsubstituted C₁-C₆ alkyl, substituted or unsubstituted C₃-C₈ cycloalkyl, substituted or unsubstituted C₂-C₇ heterocycloalkyl, substituted or unsubstituted aryl, or substituted or unsubstituted heteroaryl; wherein if R⁴¹ is substituted then it is substituted with 1, 2, or 3 substituents independently selected from fluoro, hydroxy, amino, —NH(C₁-C₄ alkyl), —N(C₁-C₄ alkyl)₂, nitro, cyano, C₁-C₄ alkyl, and C₁-C₄ alkoxy;
each X¹ and X² is independently —O—, —S—, —NR⁴⁶—, —CH₂—, or —(C═O)—;
each R⁴² and R⁴⁵ is independently deuterium, halogen, hydroxy, amino, nitro, cyano, C₁-C₆ alkyl, C₁-C₆ alkoxy, or C₁-C₆ haloalkyl;
R⁴⁴ is hydrogen, halogen, substituted or unsubstituted C₁-C₆ alkyl, substituted or unsubstituted C₁-C₆ fluoroalkyl, or substituted or unsubstituted C₁-C₆ alkoxy;
R⁴³ is substituted or unsubstituted C₁-C₆ alkyl, substituted or unsubstituted C₃-C₈ cycloalkyl, or substituted or unsubstituted C₂-C₇ heterocycloalkyl; wherein if R⁴³ is substituted then it is substituted with 1, 2, or 3 substituents independently selected from fluoro, hydroxy, amino, nitro, cyano, —N(R⁴⁶)₂, —COOR⁴⁶, —C(═O)R⁴⁶, —C(═O)NH(C₁-C₆ alkyl), —C(═O)NH(substituted or unsubstituted aryl), C₁-C₄ alkyl, C₁-C₄ alkoxy, C₂-C₆ alkenyl, C₂-C₆ alkynyl, substituted or unsubstituted C₃-C₈ cycloalkyl, substituted or unsubstituted C₂-C₇ heterocycloalkyl, substituted or unsubstituted aryl, and substituted or unsubstituted heteroaryl;
each R⁴⁶ is independently hydrogen or C₁-C₆ alkyl;
r is an integer from 0-4; and
s is an integer from 0-4.

In some embodiments, disclosed herein is a compound, or a pharmaceutically acceptable salt or solvate thereof, selected from:

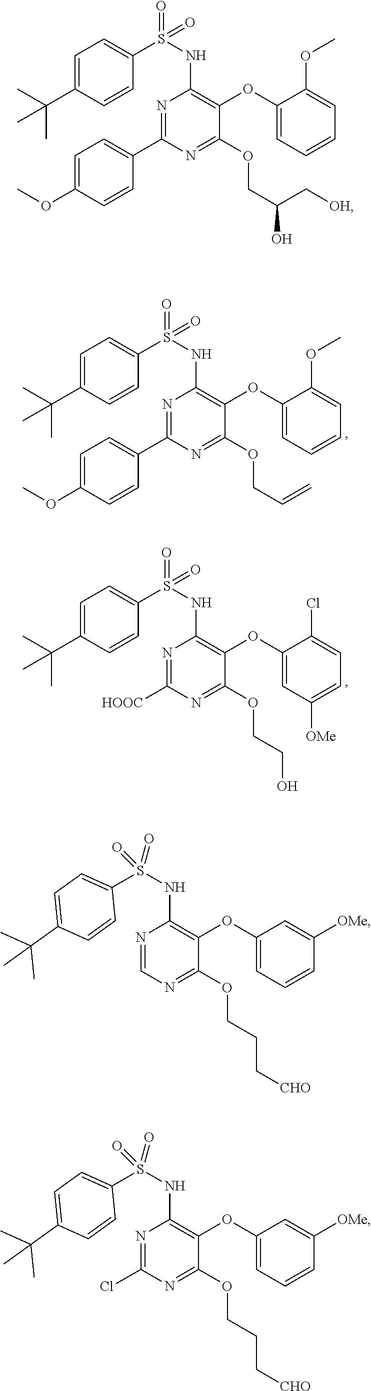

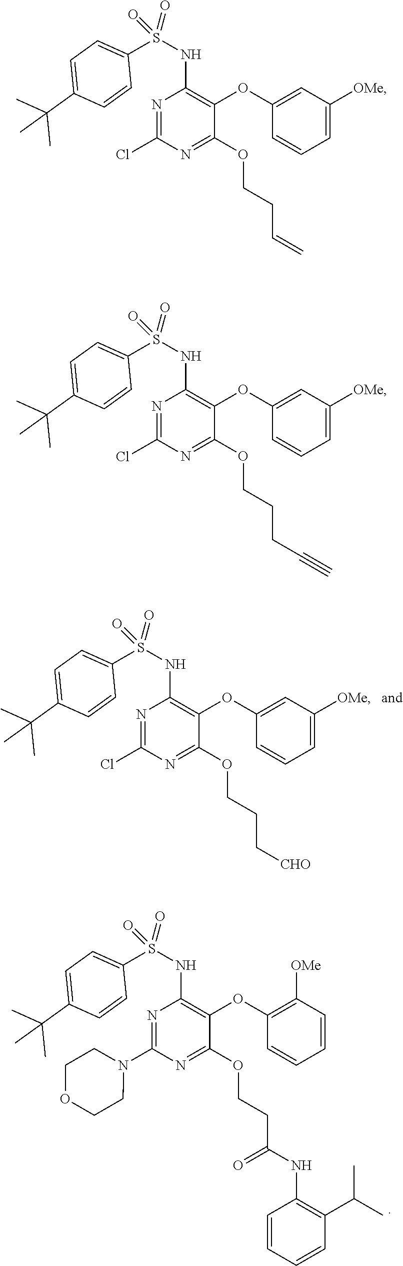 [SMILES: C=CCCOc1nc(Cl)nc(NS(=O)(=O)c2ccc(C(C)(C)C)cc2)c1Oc1cccc(OC)c1 ; C#CCCCOc1nc(Cl)nc(NS(=O)(=O)c2ccc(C(C)(C)C)cc2)c1Oc1cccc(OC)c1 ; COc1cccc(Oc2c(NS(=O)(=O)c3ccc(C(C)(C)C)cc3)nc(Cl)nc2OCCCC=O)c1 ; "and" ; COc1ccccc1Oc1c(NS(=O)(=O)c2ccc(C(C)(C)C)cc2)nc(N2CCOCC2)nc1OCCC(=O)Nc1ccccc1C(C)C]

In some embodiments, disclosed herein is a compound of Formula (8), or a pharmaceutically acceptable salt or solvate thereof:

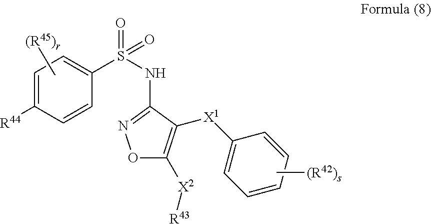

wherein,
each X¹ and X² is independently —O—, —S—, —NR⁴⁶—, —CH₂—, or —(C═O)—;
each R⁴² and R⁴⁵ is independently deuterium, halogen, hydroxy, amino, nitro, cyano, C₁-C₆ alkyl, C₁-C₆ alkoxy, or C₁-C₆ haloalkyl;
R⁴⁴ is hydrogen, halogen, substituted or unsubstituted C₁-C₆ alkyl, substituted or unsubstituted C₁-C₆ fluoroalkyl, or substituted or unsubstituted C₁-C₆ alkoxy;
R⁴³ is substituted or unsubstituted C₁-C₆ alkyl, substituted or unsubstituted C₃-C₈ cycloalkyl, or substituted or unsubstituted C₂-C₇ heterocycloalkyl; wherein if R⁴³ is substituted then it is substituted with 1, 2, or 3 substituents independently selected from fluoro, hydroxy, amino, nitro, cyano, —N(R⁴⁶)₂, —COOR⁴⁶, —C(═O)R⁴⁶, —C(═O)NH(C₁-C₆ alkyl), —C(═O)NH(substituted or unsubstituted aryl), C₁-C₄ alkyl, C₁-C₄ alkoxy, C₂-C₆ alkenyl, C₂-C₆ alkynyl, substituted or unsubstituted C₃-C₈ cycloalkyl, substituted or unsubstituted C₂-C₇ heterocycloalkyl, substituted or unsubstituted aryl, and substituted or unsubstituted heteroaryl;
each R⁴⁶ is independently hydrogen or C₁-C₆ alkyl;
r is an integer from 0-4; and
s is an integer from 0-4.

In some embodiments, disclosed herein is a compound, or a pharmaceutically acceptable salt or solvate thereof, selected from:

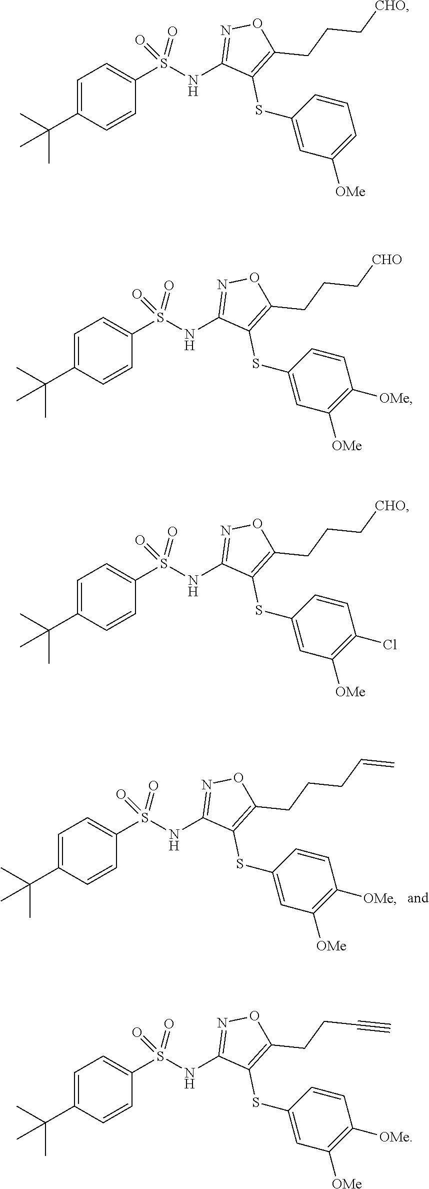

In some embodiments, disclosed herein is a compound of Formula (9), or a pharmaceutically acceptable salt or solvate thereof:

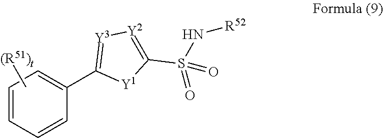

wherein,
each R⁵¹ is independently deuterium, halogen, hydroxy, nitro, cyano, —N(R⁵³)₂, —C(═O)R⁵³, —COOR⁵³, —C(═O)NHR⁵³, substituted or unsubstituted C₁-C₆ alkyl, substituted or unsubstituted C₁-C₆ alkoxy, substituted or unsubstituted C₁-C₆ haloalkyl, substituted or unsubstituted C₃-C₈ cycloalkyl, substituted or unsubstituted C₂-C₇ heterocycloalkyl, substituted or unsubstituted aryl, or substituted or unsubstituted heteroaryl; wherein if R⁵¹ is substituted then it is substituted with 1, 2, or 3 substituents independently selected from halogen, hydroxy, amino, —NH(C₁-C₄ alkyl), —N(C₁-C₄ alkyl)₂, nitro, cyano, C₁-C₄ alkyl, and C₁-C₄ alkoxy;
Y¹ is —O—, —S—, —NR⁵³—;
each Y² and Y³ is independently N or —CR⁵³—;
R⁵² is substituted or unsubstituted cycloalkyl, substituted or unsubstituted heterocycloalkyl, substituted or unsubstituted aryl, or substituted or unsubstituted heteroaryl; wherein if R⁵² is substituted then it is substituted with 1, 2, or 3 substituents independently selected from halogen, hydroxy, amino, —NH(C₁-C₄ alkyl), —N(C₁-C₄ alkyl)₂, nitro, cyano, C₁-C₄ alkyl, and C₁-C₄ alkoxy;
each R⁵³ is independently hydrogen, halogen, hydroxy, nitro, cyano, amino, C₁-C₆ alkyl, or C₁-C₄ alkoxy; and
t is an integer from 0-5.

In some embodiments, disclosed herein is a compound, or a pharmaceutically acceptable salt or solvate thereof, selected from:

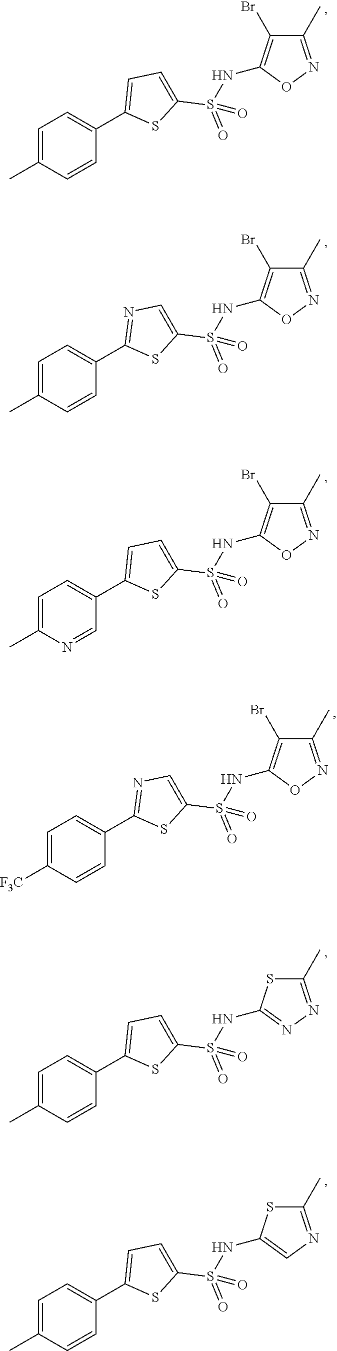

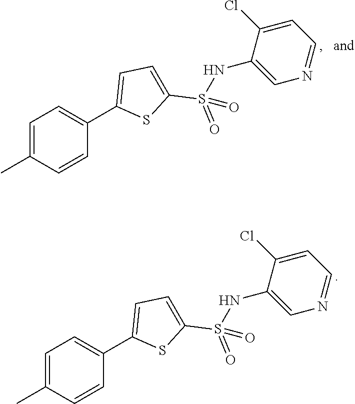 [SMILES: Cc1ccc(-c2ccc(S(=O)(=O)Nc3cnccc3Cl)s2)cc1 ; Cc1ccc(-c2ccc(S(=O)(=O)Nc3cnccc3Cl)s2)cc1]

Further Forms of Compounds

In some embodiments, a compound of Formula (1)-Formula (9) possesses one or more stereocenters and each stereocenter exists independently in either the R or S configuration. The compounds presented herein include all diastereomeric, enantiomeric, and epimeric forms as well as the appropriate mixtures thereof. The compounds and methods provided herein include all cis, trans, syn, anti, entgegen (E), and zusammen (Z) isomers as well as the appropriate mixtures thereof. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In one aspect, stereoisomers are obtained by stereoselective synthesis.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine and chlorine, such as, for example, ²H, ³H, ¹³C, ¹⁴C, ¹⁵N, ¹⁸O, ¹⁷O, ³⁵S, ¹⁸F, ³⁶C. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

The term “pharmaceutically acceptable salt” refers to a formulation of a compound that does not cause significant irritation to an organism to which it is administered and does not abrogate the biological activity and properties of the compound. In some embodiments, pharmaceutically acceptable salts are obtained by reacting a compound of Formula (1)-Formula (9) with acids. Pharmaceutically acceptable salts are also obtained by reacting a compound of Formula (1)-Formula (9) with a base to form a salt.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g. lithium, sodium, potassium), an alkaline earth ion (e.g. magnesium, or calcium), or an aluminum ion. In some cases, compounds described herein may coordinate with an organic base, such as, but not limited to, ethanolamine, diethanolamine, triethanolamine, tromethamine, N-methylglucamine, dicyclohexylamine, tris(hydroxymethyl)methylamine. In other cases, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms, particularly solvates. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

Pharmaceutical Compositions

Provided herein are pharmaceutical compositions comprising at least one ETBR antagonist of formula (4), formula (5), formula (6), formula (7), formula (8) or formula (9), and a pharmaceutically acceptable carrier. In some embodiments, the compositions herein are formulated in a unit dosage form, including any desired carrier or excipient, and configured for administration via any desired route, e.g., oral, intravenous, subcutaneous, intramuscular, intraperitoneal, parenteral, intranasal, intracranial. In some embodiments, the compositions as described herein are useful for the treatment of ETBR-related cancer in a patient. In some embodiments, the cancer is a solid tumor. In some embodiments, the cancer is at least one of breast cancer, melanoma, SCC, glioblastoma, ovarian cancer, pancreatic cancer, or a combination thereof. In some embodiments, the compositions comprise a polymorph of an ETBR antagonist. In some embodiments, the compositions comprise a dosage of the ETBR antagonist of about 0.1 mg to about 500 mg (e.g., about 10 mg to about 100 mg), and/or a concentration of the ETBR antagonist of about 0.01 g/mL to about 1000 mg/mL (e.g., about 0.1 mg/mL to about 5 mg/mL).

In some embodiments, the compositions as described herein are formulated in a conventional manner using one or more pharmaceutically acceptable carriers or excipients and may also be administered in controlled-release formulations. Pharmaceutically acceptable carriers or excipients that may be used in these pharmaceutical compositions include, but are not limited to, dimethyl sulfoxide (DMSO), LYOCELL (reversed cubic phase liquid crystal dispersion), INTRAVAIL (transmucosal absorption enhancement agents), PROTEK (protein stabilization excipients), or hydrogel, soybean oil, or any combination thereof as a carrier, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

In some embodiments, the compositions include at least one of soybean oil, dimethyl sulfoxide (DMSO), hydrogel, LYOCELL (reversed cubic phase liquid crystal dispersion), soybean oil, INTRAVAIL (transmucosal absorption enhancement agents), PROTEK (protein stabilization excipients), or hydrogel, or any combination thereof or a combination thereof. Any of the embodiments described herein can be a single-component oil phase formulation, as described above, wherein each active ingredient can be at any of the dosages or concentrations described herein. The single-component oil phase can be a fixed oil, such as soybean oil. For example, the formulation can comprise about 0.1 mg to about 5.0 mg of each active ingredient in 1 mL of the single-component oil (i.e., about 0.5 mg/mL, about 1 mg/mL, or about 1.5 mg/mL of each active ingredient in the single-component oil). The single-component oil phase formulation can be prepared by adding each active ingredient (e.g., about 1 mg to about 50 mg of each of the active ingredient(s)) to about 10 mL of the single-component oil solution.

In some embodiments, pharmaceutical compositions herein comprise a DMSO, e.g., in a DMSO solution that is about 5% to about 100% DMSO (e.g., about 10% to about 100%, about 20% to about 100%, about 30% to about 100%, about 40% to about 100%, about 50% to about 100%, about 60% to about 100%, about 70% to about 100%, about 80% to about 100%, about 90% to about 100%, about 30% to about 95%, about 45% to about 95%, about 75% to about 95%, about 30% to about 90%, about 45% to about 90%, about 75% to about 90%, about 30% to about 85%, about 45% to about 85%, or about 75% to about 85%). For example, the pharmaceutical compositions can comprise about 0.1 mg to about 5.0 mg of each active ingredient in 1 mL of DMSO (i.e., about 0.5 mg/mL, about 1 mg/mL, or about 1.5 mg/mL of each active ingredient in DMSO). The DMSO pharmaceutical compositions can be prepared by adding each active ingredient (e.g., about 1 mg to about 50 mg of each of the active ingredient(s)) to about 10 mL of the DMSO solution. For example, the DMSO is a DMSO solution comprising about 5% to about 100% DMSO, about 25% to about 100% DMSO, about 50% to about 100% DMSO, about 75% to about 100% DMSO, about 5% to about 75% DMSO, about 25% to about 75% DMSO, about 50% to about 75% DMSO, about 5% to about 50% DMSO, about 25% to about 50% DMSO, or about 5% to about 25% DMSO.

In some embodiments, the description provides a controlled release subcutaneous or intramuscular dosage formulation comprising a uniform dispersion of an ETBR antagonist (e.g., BQ-788, A192621, A-308165, IRL-1038, IRL-2500, RO-468443, BQ-017, a structural analog such as a deuterated or fluorinated analog thereof, or combinations thereof) and an ETAR antagonist (e.g., BQ123) in a biocompatible delivery system whereby following administration the ETBR and ETAR antagonists are released slowly and simultaneously from the formulation into the systemic circulation.

In some embodiments, the pharmaceutical composition as described herein is formulated into a controlled release delivery system comprising at least one biocompatible polymer. In some embodiments, the active compounds are prepared with carriers that will protect the compound against rapid elimination from the body, such as a controlled release formulation, including implants, hydrogels, thermo-sensitive hydrogels, and microencapsulated delivery systems. Biodegradable, biocompatible polymers can be used, such as ethylene vinyl acetate, acrylates, polycarboxylic acids, polyanhydrides, polyglycolic acid, collagen, polyorthoesters, and polylactic acid. In some embodiments, the biocompatible polymer is at least one of a poly(lactide), poly(glycolide), poly(lactide-co-glycolide), poly(lactic acid), poly(glycolic acid), poly(lactic acid-co-glycolic acid), polycaprolactone, polycarbonate, polyesteramide, polyanhydride, poly(amino acid), polyorthoester, polycyanoacrylate, poly(p-dioxanone), poly(alkylene oxalate), biodegradable polyurethane, blend, or a copolymer thereof.

In some embodiments, the pharmaceutically acceptable carrier comprises or is a liposome. For example, the pharmaceutical composition or formulation may comprise a liposome having an interior volume comprising an ETBR antagonist. In some embodiments, the liposome is configured to effectuate the controlled release of the ETBR antagonist, e.g., rapid release, extended release, or a combination thereof.

In some embodiments, the liposome is configured to effectuate the controlled release of the pharmaceutical compositions. In some embodiments, the liposome is configured to effectuate rapid release of the pharmaceutical compositions. In other embodiments, the liposome is configured or formulated to effectuate extended release the pharmaceutical compositions. In some embodiments, the liposome is configured to result in both the rapid and extended release of pharmaceutical compositions.

In some embodiments, the liposome is configured to effectuate the controlled release of the ETBR antagonist or the caspase-8 inhibitor or a combination thereof. In some embodiments, the liposome is configured to effectuate rapid release of the ETBR antagonist or the caspase-8 inhibitor or a combination thereof. In other embodiments, the liposome is configured or formulated to effectuate extended release the ETBR antagonist or the caspase-8 inhibitor or a combination thereof. In some embodiments, the liposome is configured to result in both the rapid and extended release of the ETBR antagonist or the caspase-8 inhibitor or a combination thereof.

In some embodiments, liposomal suspensions are pharmaceutically acceptable carriers. For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound is then introduced into the container. The container is then swirled by hand to free lipid material from the sides of the container and to disperse lipid aggregates, thereby forming the liposomal suspension.

In some embodiments, the pharmaceutical compositions comprise a liposome having an interior volume comprising an ETBR antagonist or a caspase-8 inhibitor or a combination thereof, and an effective amount of at least one of an ETAR antagonist, an anti-PD1 antibody, a bRAF inhibitor, niacinamide or a combination thereof. In some embodiments, the liposome comprises at least one of a neutral lipid, a basic (having a net positive charge) lipid, an acidic (having a net negative charge) lipid, cholesterol, or a combination thereof. In some embodiments, the liposome further comprises a polymeric component. In some embodiments, the interior volume of the liposome is at least partially aqueous, and comprises an ETBR antagonist.

In some embodiments, the description provides the pharmaceutical composition as described herein in a liposomal delivery system, e.g., at least one of a phosphatidylethanolamine (PE) such as dipalmitoyl PE (DPPE), and partially unsaturated phosphatidylcholine (PC), such as egg PC (EPC) or SPC, fully unsaturated PC such as HSPC, PG, phosphatidylserine (PS), phosphatidylinositol (PI) or a combination thereof. In some embodiments, the phospholipid is at least one of a partially unsaturated PG, dipalmitoylphosphatidylglycerol (DPPG), cholesterol, DSPE-PEG2000, polysorbate-80 or combination thereof. In some embodiments, the liposomal delivery system is a controlled release system, e.g., at least one of rapid release, extended release, rapid and extended release, delayed release, sustained release, slow release, and combinations thereof.

In some embodiments, the pharmaceutical compositions herein comprise pharmaceutically acceptable salts, in particular, acid or base addition salts of compounds as described herein. The acids which are used to prepare the pharmaceutically acceptable acid addition salts of the aforementioned base compounds useful according to this aspect are those which form non-toxic acid addition salts, i.e., salts containing pharmacologically acceptable anions, such as the hydrochloride, hydrobromide, hydroiodide, nitrate, sulfate, bisulfate, bitartrate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3 naphthoate)]salts, among numerous others. Pharmaceutically acceptable base addition salts may also be used to produce pharmaceutically acceptable salt forms of the compounds or derivatives according to the present disclosure. The chemical bases that may be used as reagents to prepare pharmaceutically acceptable base salts of the present compounds that are acidic in nature are those that form nontoxic base salts with such compounds. Such non-toxic base salts include, but are not limited to those derived from such pharmacologically acceptable cations such as alkali metal cations (eg., potassium and sodium) and alkaline earth metal cations (e.g., calcium, zinc and magnesium), ammonium or water-soluble amine addition salts such as N-methylglucamine-(meglumine), and the lower alkanolammonium and other base salts of pharmaceutically acceptable organic amines, among others.

In some embodiments, compositions include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or enteric agents.

In some embodiments, the active compound or pharmaceutically acceptable salt thereof is administered as a component of an elixir, suspension, syrup, wafer, chewing gum or the like. A syrup may contain, in addition to the active compounds, sucrose as a sweetening agent and certain preservatives, dyes and colorings and flavors.

In some embodiments, solutions or suspensions used for parenteral, intradermal, subcutaneous, intravenous, intramuscular, or topical application include the following components: a sterile diluent such as water for injection, saline solution, fixed oils (e.g., soybean oil), polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic. In some embodiments, carriers for intravenous administration are physiological saline or phosphate buffered saline (PBS).

Combination Therapy

Disclosed herein are pharmaceutical compositions for therapeutic combinations, in a single dosage form or separate dosage forms administered concurrently or separately, comprising at least one ETBR antagonist of formula (4), formula (5), formula (6), formula (7), formula (8) or formula (9), and at least one additional anti-oncologic agent. In some embodiments, the at least one additional anti-oncologic agent is an immune checkpoint inhibitor, e.g., an anti-PD1 antibody or anti-PD-L1 antibody. In some embodiments, the ETBR antagonist is administered 2, 3, 4, or 5 times frequently as the additional anti-oncologic agent, for example that the ETBR antagonist is administered 3 times during 1-3 weeks (e.g, about 2-3 weeks or about 21 days) while the additional anti-oncologic agent is administered 1 time during the 1-3 weeks (e.g., about 2-3 weeks or about 21 days).

In some embodiments, the description provides pharmaceutical compositions comprising a first composition comprising an ETBR antagonist of formula (4), formula (5), formula (6), formula (7), formula (8) or formula (9) in an amount effective when administered with at least one additional anticancer or anti-oncologic agent; and a second composition comprising an effective amount of the at least one additional anticancer or anti-oncologic agent as described herein.

In some embodiments, the description provides a combination comprising at least one ETBR antagonist of formula (4), formula (5), formula (6), formula (7), formula (8) or formula (9), and at least one additional anti-oncologic therapeutic agent. In some embodiments, the at least one anti-oncologic agent is a bRaf inhibitor, an immune checkpoint inhibitor, a caspase-8 inhibitor, an ETAR antagonist, niacinamide, a chemotherapeutic agent such as, e.g., a taxane, a kinase inhibitor, or other receptor antagonist or combination thereof. In some embodiments, the pharmaceutical compositions comprise an effective amount of at least two of ETBR antagonist, bRaf inhibitor, an immune checkpoint inhibitor, a caspase-8 inhibitor, an ETAR antagonist, niacinamide, a chemotherapeutic agent such as, e.g., a taxane, a kinase inhibitor, or other receptor antagonist or combination thereof.

In some embodiments, the ETBR antagonist and the at least one additional anti-oncologic therapeutic agent are separate pharmaceutical compositions. In some embodiments, the ETBR antagonist and the at least one additional anti-oncologic therapeutic agent are comprised in the same pharmaceutical composition.

In some embodiments, the description provides methods comprising administering an ETBR antagonist of formula (4), formula (5), formula (6), formula (7), formula (8) or formula (9) in an amount effective for treating cancer and an anti-oncologic agent, and a pharmaceutically acceptable excipient or carrier.

In some embodiments, the description provides a pharmaceutical composition comprising an ETBR antagonist of formula (4), formula (5), formula (6), formula (7), formula (8) or formula (9) in an amount effective for treating cancer, and a pharmaceutically acceptable carrier. In some embodiments, the amount is effective to treat cancer when also administered with at least one additional anti-oncologic agent, and a pharmaceutically acceptable excipient or carrier. In some embodiments, the description provides a therapeutic combination comprising, in the same or separate dosage forms, an effective amount of the at least one ETBR antagonist of formula (4), formula (5), formula (6), formula (7), formula (8) or formula (9) and an effective amount of at least one anti-oncologic agent. In some embodiments, the combination includes a pharmaceutical acceptable carrier. In some embodiments, the combination or formulation is comprised in one or more unit dosage forms. In further embodiments, the combination is comprised in separate unit dosage forms, for example, a first container comprising the at least one ETBR antagonist, and a second container comprising the at least one anti-oncologic agent.

In some embodiments, the at least one anti-oncologic agent is an immune checkpoint inhibitor. In some embodiments, the immune checkpoint inhibitor is an anti-PD1 antibody or an anti-PD-L1 antibody. In some embodiments, the anti-PD1 antibody is at least one of nivolumab, pembrolizumab, pidilizumab, or any combination thereof. In some embodiments, the anti-PD-L1 antibody is atezolizumab, MDX-1105, avelumab, durvalumab, or any combination thereof.

In some embodiments, the bRAF inhibitor is at least one of dabrafenib, sorafenib, or vemurafenib, or any combination thereof.

In some embodiments, caspase-8 is a downstream effector of the ETBR, and caspase-8 inhibitors block molecular events that promote invasion and metastasis that are triggered as a result of ETBR activation. As such, caspase-8 inhibitors can be classified as a caspase-8 antagonist or an antagonist/inhibitor of ETBR signaling. In some embodiments, the caspase-8 inhibitor peptide has a sequence of Ac-AAVALLPAVLLAALAPIETD-CHO (SEQ ID NO:1), which is commercially available from EMD Millipore (Billerica, Mass. 01821, USA).

In some embodiments, the physiologic role of the ETBR is to clear excess levels of endothelin-1 (ET-1), from the circulation. Without being bound by any particular theory, it is hypothesized that administering an ETBR antagonist prevents ET-1 clearance and elevates serum ET-1 levels. Elevated serum levels of ET-1 are associated with a variety of adverse effects due to its activation of the Endothelin A receptor (ETAR) including, hypertension, pulmonary hypertension and renal vasoconstriction. In some embodiments, in order to minimize the unwanted effect of ETAR activation, the description provides pharmaceutical compositions and methods for combination therapy (in a single dosage form or separate dosage forms administered approximately contemporaneously) of an ETBR antagonist with an ETAR antagonist. The formulations as described herein are useful for the treatment of cancer in a patient, for example, breast cancer, melanoma, SCC, glioblastoma; solid tumors or a combination thereof.

In some embodiments, the ETAR antagonist is BQ123. BQ123 (2-[(3R,6R,9S,12R,15S)-6-(1H-indol-3-ylmethyl)-9-(2-methylpropyl)-2,5,8,11,14-pentaoxo-12-propan-2-yl-1,4,7,10,13-pentazabicyclo[13.3.0]octadecan-3-yl]acetic acid or cyclo(D-Trp-D-Asp-Pro-D-Val-Leu)) is a selective ETAR antagonist.

In some embodiments, pharmaceutical compositions herein comprise an effective amount of an ETBR antagonist of formula (4), formula (5), formula (6), formula (7), formula (8) or formula (9) in combination with an effective amount of an ETAR antagonist, and a pharmaceutically acceptable carrier. In some embodiments, the ETAR antagonist is BQ123, including analogs, derivatives, polymorphs, prodrugs, and salts thereof.

In some embodiments, the additional anti-oncologic agent is at least one of apx005m, ipilimumab, vemurafenib, dacarbazine, nivolumab, pembrolizumab, niacinamide, interleukin-2, DEDN6526, Talimogene laherparepvec, tumor infiltrating lymphocytes, an anti-angiogenic agent, adriamycin, camptothecin, carboplatin, cisplatin, daunorubicin, doxorubicin, alpha, beta, or gamma interferon, irinotecan, docetaxel, paclitaxel, topotecan, atrasentan, tezosentan, bosentan, sitaxsentan, enrasentan, zibotentan, Ro468443, TBC10950, TBC10894, A192621, A308165, SB209670, SB17242, A182086, (s)-Lu302872, J-104132, TAK-044, Sarafotoxin 56c, IRL2500, RES7011, Aselacins A, B, and C, Ro470203, Ro462005, sulfamethoxazole, cochinmicin I, II, and III, L749329, L571281, L754142, J104132, CGS27830, PD142893, PD143296, PD145065, PD156252, PD159020, PD160672, PD160874, TM-ET-1, IRL3630, Ro485695, L75037, LU224332, PD142893, LU302872, PD145065, Ro610612, SB217242, or a combinations thereof. In some embodiments, the additional anti-oncologic agent is a RAF kinase antagonist, a MEK antagonist or a combination thereof. In some embodiments, the anti-oncologic agent is at least one of an IDO inhibitor, HDAC inhibitor, DNMT inhibitor, adenosine receptor inhibitor, CXCR4/CXCL12 axis inhibitor or a combination thereof. In some embodiments, the DNMT inhibitor is vidaza. In some embodiments, the HDAC inhibitor is at least one of entinostat, mocetinostat, inostat, romidepsin, ACY-241, farydak or a combination thereof. In some embodiments, the adenosine receptor inhibitor is at least one of CPI-444 (V81444), PBF-509, MEDI9447, MK-3814, AZD4635, BMS-986179 or a combination thereof. In some embodiments, the CXCR4/CXCL12 axis inhibitor is at least one of ulocuplumab, BL-8040, PF-06747143, POL6326, plerixafor, ALX-0651, LY2510924, AMD11070, X4P-001, Q122, USL311, burixafor hyrobromid, CX-01, CTCE 9908, GMI-1359 or a combination thereof. In some embodiments, the anti-oncologic agent is an anti-angiogenic agent selected from thalidomide, marimastat, COL-3, BMS275291, squalamine, 2-ME, SU6668, neovastat, Medi522, EMD121974, CAI, celecoxib, interleukin-12, IM862, TNP470, avastin, gleevac, herceptin, or a combination thereof. In some embodiments, the anti-oncologic agent is a cell CDK4/6 cycle inhibitor, for example, ribociclib, palbociclib, milciclib, voruciclib, abemaciclib, flavopiridol or a combination thereof.

In some embodiments, a dosage of the ETBR antagonist is about 0.1 μg to about 500 mg (e.g., about 100 μg to about 4000 μg) and/or a concentration of the ETBR antagonist is about 0.01 μg/mL to about 1000 mg/mL of the composition (e.g., about 0.1 mg/mL to about 5 mg/mL).

In some embodiments, a dosage of the ETAR antagonist is about 0.1 μg to about 500 mg (e.g., about 100 μg to about 4000 μg) and/or a concentration of the ETAR antagonist is about 0.01 μg/mL to about 1000 mg/mL of the composition (e.g., about 0.1 mg/mL to about 5 mg/mL).

In some embodiments, a dosage of the anti-PD1 antibody is about 0.1 μg to about 500 mg (e.g., about 100 μg to about 4000 μg) and/or a concentration of the anti-PD1 antibody is about 0.01 μg/mL to about 1000 mg/mL of the composition (e.g., about 0.1 mg/mL to about 5 mg/mL).

In some embodiments, a dosage of the bRAF inhibitor is about 0.1 μg to about 500 mg (e.g., about 100 μg to about 4000 μg) and/or a concentration of the bRAF inhibitor is about 0.01 μg/mL to about 1000 mg/mL of the composition (e.g., about 0.1 mg/mL to about 5 mg/mL).

In some embodiments, a dosage of the niacinamide is about 0.1 μg to about 500 mg (e.g., about 100 μg to about 4000 μg) and/or a concentration of the niacinamide is about 0.01 μg/mL to about 1000 mg/mL of the composition (e.g., about 0.1 mg/mL to about 5 mg/mL).

In some embodiments, a dosage of the caspase-8 inhibitor is about 0.1 μg to about 500 mg (e.g., about 100 μg to about 4000 μg or about 1 μg to about 4000 μg) and/or a concentration of the caspase-8 inhibitor is about 0.01 μg/mL to about 1000 mg/mL of the composition (e.g., about 0.1 mg/mL to about 5 mg/mL).

In some embodiments, the concentration of the at least one ETBR antagonist, and/or the at least one anti-oncologic agent can independently be about 0.01 μg/mL to about 1000 mg/mL, about 0.01 μg/mL to about 750 mg/mL, about 0.01 μg/mL to about 500 mg/mL, about 0.01 μg/mL to about 300 mg/mL, about 0.01 μg/mL to about 150 mg/mL, about 0.01 μg/mL to about 100 mg/mL, about 0.01 μg/mL to about 50 mg/mL, about 0.01 μg/mL to about 25 mg/mL, about 0.01 μg/mL to about 10 mg/mL, about 0.01 μg/mL to about 1.0 mg/mL, about 0.01 μg/mL to about 0.1 μg/mL, about 0.1 μg/mL to about 750 mg/mL, about 0.1 μg/mL to about 500 mg/mL, about 0.1 μg/mL to about 300 mg/mL, about 0.1 μg/mL to about 150 mg/mL, about 0.1 μg/mL to about 100 mg/mL, about 0.1 μg/mL to about 50 mg/mL, about 0.1 μg/mL to about 25 mg/mL, about 0.1 μg/mL to about 10 mg/mL, about 0.1 μg/mL to about 1.0 mg/mL, about 1.0 μg/mL to about 750 mg/mL, about 1.0 μg/mL to about 500 mg/mL, about 1.0 μg/mL to about 300 mg/mL, about 1.0 μg/mL to about 150 mg/mL, about 1.0 μg/mL to about 100 mg/mL, about 1.0 μg/mL to about 50 mg/mL, about 1.0 μg/mL to about 25 mg/mL, about 1.0 μg/mL to about 10 mg/mL, about 10 μg/mL to about 750 mg/mL, about 10 μg/mL to about 500 mg/mL, about 10 μg/mL to about 300 mg/mL, about 10 μg/mL to about 150 mg/mL, about 10 μg/mL to about 100 mg/mL, about 10 μg/mL to about 50 mg/mL, about 10 μg/mL to about 25 mg/mL, about 25 μg/mL to about 750 mg/mL, about 25 μg/mL to about 500 mg/mL, about 25 μg/mL to about 300 mg/mL, about 25 μg/mL to about 150 mg/mL, about 25 μg/mL to about 100 mg/mL, about 25 μg/mL to about 50 mg/mL, about 50 μg/mL to about 750 mg/mL, about 50 μg/mL to about 500 mg/mL, about 50 μg/mL to about 300 mg/mL, about 50 μg/mL to about 150 mg/mL, about 50 μg/mL to about 100 mg/mL, about 100 μg/mL to about 750 mg/mL, about 100 μg/mL to about 500 mg/mL, about 100 μg/mL to about 300 mg/mL, about 100 μg/mL to about 150 mg/mL, about 150 μg/mL to about 750 mg/mL, about 150 μg/mL to about 500 mg/mL, about 150 μg/mL to about 300 mg/mL, about 300 μg/mL to about 750 mg/mL, about 300 μg/mL to about 500 mg/mL, or about 500 μg/mL to about 750 mg/mL.

In some embodiments, the dosage of the at least one ETBR antagonist, and/or at least one anti-oncologic agent can independently be about 0.1 μg to about 5000 μg, about 0.1 μg to about 4500 μg, about 0.1 μg to about 4000 μg, about 0.1 μg to about 3500 μg, about 0.1 μg to about 3000 μg, about 0.1 μg to about 2500 μg, about 0.1 μg to about 2000 μg, about 0.1 μg to about 1500 μg, about 0.1 μg to about 1000 μg, about 0.1 μg to about 500 μg, about 1.0 μg to about 5000 μg, about 1.0 μg to about 4500 μg, about 1.0 μg to about 4000 μg, about 1.0 μg to about 3500 μg, about 1.0 μg to about 3000 μg, about 1.0 μg to about 2500 μg, about 1.0 μg to about 2000 μg, about 1.0 μg to about 1500 μg, about 1.0 g to about 1000 μg, about 1.0 μg to about 500 μg, about 100 μg to about 5000 μg, about 100 μg to about 4500 μg, about 100 μg to about 4000 μg, about 100 μg to about 3500 μg, about 100 μg to about 3000 μg, about 100 μg to about 2500 μg, about 100 μg to about 2000 μg, about 100 μg to about 1500 μg, about 100 μg to about 1000 μg, about 100 μg to about 500 μg, about 250 μg to about 5000 μg, about 250 μg to about 4500 μg, about 250 μg to about 4000 μg, about 250 μg to about 3500 μg, about 250 μg to about 3000 μg, about 250 μg to about 2500 μg, about 250 μg to about 2000 μg, about 250 μg to about 1500 μg, about 250 μg to about 1000 μg, about 250 μg to about 500 μg, about 500 μg to about 5000 μg, about 500 μg to about 4500 μg, about 500 μg to about 4000 μg, about 500 μg to about 3500 μg, about 500 μg to about 3000 μg, about 500 μg to about 2500 μg, about 500 μg to about 2000 μg, about 500 μg to about 1500 μg, about 500 μg to about 1000 μg, about 750 μg to about 5000 μg, about 750 μg to about 4500 μg, about 750 μg to about 4000 μg, about 750 μg to about 3500 μg, about 750 μg to about 3000 μg, about 750 μg to about 2500 μg, about 750 μg to about 2000 μg, about 75 μg to about 1500 μg, about 750 μg to about 1000 μg, about 1500 μg to about 5000 μg, about 1500 μg to about 4500 μg, about 1500 μg to about 4000 μg, about 1500 μg to about 3500 μg, about 1500 μg to about 3000 μg, about 1500 μg to about 2500 μg, about 1500 μg to about 2000 μg, about 2000 μg to about 5000 μg, about 2000 μg to about 4500 μg, about 2000 μg to about 4000 μg, about 2000 μg to about 3500 μg, about 2000 μg to about 3000 μg, about 2000 μg to about 2500 μg, about 2500 μg to about 5000 μg, about 2500 μg to about 4500 μg, about 2500 μg to about 4000 μg, about 2500 μg to about 3500 μg, about 2500 μg to about 3000 μg, about 3000 μg to about 5000 μg, about 3000 μg to about 4500 μg, about 3500 μg to about 4000 μg, about 3500 μg to about 5000 μg, about 3500 μg to about 4500 μg, about 3500 μg to about 4000 μg, about 4000 μg to about 5000 μg, about 4000 μg to about 4500 μg, or about 4500 μg to about 5000 μg.

In some embodiments, a dosage of the anti-PD1 antibody is about 0.1 mg/kg to about 9.0 mg/kg. For example, the dosage of the anti-PD1 antibody is about 0.1 mg/kg to about 9.0 mg/kg, about 0.1 mg/kg to about 8.0 mg/kg, about 0.1 mg/kg to about 7.0 mg/kg, about 0.1 mg/kg to about 6.0 mg/kg, about 0.1 mg/kg to about 5.0 mg/kg, about 0.1 mg/kg to about 4.0 mg/kg, about 0.1 mg/kg to about 3.0 mg/kg, about 0.1 mg/kg to about 2.0 mg/kg, about 0.1 mg/kg to about 1.0 mg/kg, about 1.0 mg/kg to about 9.0 mg/kg, about 1.0 mg/kg to about 8.0 mg/kg, about 1.0 mg/kg to about 7.0 mg/kg, about 1.0 mg/kg to about 6.0 mg/kg, about 1.0 mg/kg to about 5.0 mg/kg, about 1.0 mg/kg to about 4.0 mg/kg, about 1.0 mg/kg to about 3.0 mg/kg, about 1.0 mg/kg to about 2.0 mg/kg, about 2.0 mg/kg to about 9.0 mg/kg, about 2.0 mg/kg to about 8.0 mg/kg, about 2.0 mg/kg to about 7.0 mg/kg, about 2.0 mg/kg to about 6.0 mg/kg, about 2.0 mg/kg to about 5.0 mg/kg, about 2.0 mg/kg to about 4.0 mg/kg, about 2.0 mg/kg to about 3.0 mg/kg, about 3.0 mg/kg to about 9.0 mg/kg, about 3.0 mg/kg to about 8.0 mg/kg, about 3.0 mg/kg to about 7.0 mg/kg, about 3.0 mg/kg to about 6.0 mg/kg, about 3.0 mg/kg to about 5.0 mg/kg, about 3.0 mg/kg to about 4.0 mg/kg, about 4.0 mg/kg to about 9.0 mg/kg, about 4.0 mg/kg to about 8.0 mg/kg, about 4.0 mg/kg to about 7.0 mg/kg, about 4.0 mg/kg to about 6.0 mg/kg, about 4.0 mg/kg to about 5.0 mg/kg, about 5.0 mg/kg to about 9.0 mg/kg, about 5.0 mg/kg to about 8.0 mg/kg, about 5.0 mg/kg to about 7.0 mg/kg, about 5.0 mg/kg to about 6.0 mg/kg, about 6.0 mg/kg to about 9.0 mg/kg, about 6.0 mg/kg to about 8.0 mg/kg, about 6.0 mg/kg to about 7.0 mg/kg, about 7.0 mg/kg to about 9.0 mg/kg, about 7.0 mg/kg to about 8.0 mg/kg, or about 8.0 mg/kg to about 9.0 mg/kg.

In some embodiments, a dosage of the bRAF inhibitor is about 1 mg to about 1500 mg. For example, the dosage of the bRAF inhibitor about 1 mg to about 1500 mg, about 1 mg to about 1250 mg, about 1 mg to about 1000 mg, about 1 mg to about 750 mg, about 1 mg to about 500 mg, about 1 mg to about 250 mg, about 250 mg to about 1500 mg, about 250 mg to about 1250 mg, about 250 mg to about 1000 mg, about 250 mg to about 750 mg, about 250 mg to about 500 mg, about 500 mg to about 1500 mg, about 500 mg to about 1250 mg, about 500 mg to about 1000 mg, about 500 mg to about 750 mg, about 750 mg to about 1500 mg, about 750 mg to about 1250 mg, about 750 mg to about 1000 mg, about 1000 mg to about 1500 mg, about 1000 mg to about 1250 mg, or about 1250 mg to about 1500 mg.

In some embodiments, a dosage of the niacinamide is about 1 mg to about 3000 mg. For example, the dosage of the niacinamide is about 1 mg to about 3000 mg, about 1 mg to about 2750 mg, about 1 mg to about 2500 mg, about 1 mg to about 2250 mg, about 1 mg to about 2000 mg, about 1 mg to about 1750 mg, about 1 mg to about 1500 mg, about 1 mg to about 1250 mg, about 1 mg to about 1000 mg, about 1 mg to about 750 mg, about 1 mg to about 500 mg, about 1 mg to about 250 mg, about 250 mg to about 3000 mg, about 250 mg to about 2750 mg, about 250 mg to about 2500 mg, about 250 mg to about 2250 mg, about 250 mg to about 2000 mg, about 250 mg to about 1750 mg, about 250 mg to about 1500 mg, about 250 mg to about 1250 mg, about 250 mg to about 1000 mg, about 250 mg to about 750 mg, about 250 mg to about 500 mg, about 500 mg to about 3000 mg, about 500 mg to about 2750 mg, about 500 mg to about 2500 mg, about 500 mg to about 2250 mg, about 500 mg to about 2000 mg, about 500 mg to about 1750 mg, about 500 mg to about 1500 mg, about 500 mg to about 1250 mg, about 500 mg to about 1000 mg, about 500 mg to about 750 mg, about 750 mg to about 3000 mg, about 750 mg to about 2750 mg, about 750 mg to about 2500 mg, about 750 mg to about 2250 mg, about 750 mg to about 2000 mg, about 750 mg to about 1750 mg, about 750 mg to about 1500 mg, about 750 mg to about 1250 mg, about 750 mg to about 1000 mg, about 1000 mg to about 3000 mg, about 1000 mg to about 2750 mg, about 1000 mg to about 2500 mg, about 1000 mg to about 2250 mg, about 1000 mg to about 2000 mg, about 1000 mg to about 1750 mg, about 1000 mg to about 1500 mg, about 100 mg to about 1250 mg, about 1250 mg to about 3000 mg, about 1250 mg to about 2750 mg, about 1250 mg to about 2500 mg, about 1250 mg to about 2250 mg, about 1250 mg to about 2000 mg, about 1250 mg to about 1750 mg, about 1250 mg to about 1500 mg, about 1500 mg to about 3000 mg, about 1500 mg to about 2750 mg, about 1500 mg to about 2500 mg, about 1500 mg to about 2250 mg, about 1500 mg to about 2000 mg, about 1500 mg to about 1750 mg, about 1750 mg to about 3000 mg, about 1750 mg to about 2750 mg, about 1750 mg to about 2500 mg, about 1750 mg to about 2250 mg, about 1750 mg to about 2000 mg, about 2000 mg to about 3000 mg, about 2000 mg to about 2750 mg, about 2000 mg to about 2500 mg, about 2000 mg to about 2250 mg, about 2250 mg to about 3000 mg, about 2250 mg to about 2750 mg, about 2250 mg to about 2500 mg, about 2500 mg to about 3000 mg, about 2500 mg to about 2750 mg, or about 2750 mg to about 3000 mg.

Kits

Disclosed herein is a kit or pharmaceutical compositions for treatment of a solid tumor cancer in a subject, e.g., a human subject, comprising at least one ETBR antagonist in an amount effective for use in a combination therapy with at least one immune checkpoint inhibitor, and a pharmaceutically acceptable carrier. In some embodiments, the at least one ETBR antagonist is at least one ETBR antagonist, e.g., an analog of BQ-788, A192621, A-308165, IRL-1038, IRL-2500, RO-468443, BQ-017, as described herein. In some embodiments, the at least one ETBR antagonist is disposed in a single container with the immune checkpoint inhibitor. In some embodiments, the at least one ETBR antagonist is disposed in a first container, and the immune checkpoint inhibitor is disposed in a second container, wherein the at least one ETBR antagonist and the immune checkpoint inhibitor are to be administered approximately contemporaneously.

In some embodiments, the description provides a kit for treatment of a solid tumor cancer in a human subject, comprising an amount of at least one immune checkpoint inhibitor, an ETBR antagonist for example an analog of BQ-788, A192621, A-308165, IRL-1038, IRL-2500, RO-468443, BQ-017, and a pharmaceutically acceptable carrier or excipient for example dimethyl sulfoxide (DMSO), LYOCELL (reversed cubic phase liquid crystal dispersion), soybean oil, INTRAVAIL (transmucosal absorption enhancement agents), PROTEK (protein stabilization excipients), or hydrogel, or any combination thereof. In some embodiments, the at least one checkpoint inhibitor is an anti-PD1 antibody or anti-PD-L1 antibody.

Routes of Administration

Disclosed herein is a variety of routes of administration for the pharmaceutical compositions disclosed herein. The compounds as described herein may, in accordance with the disclosure, be administered in single or divided doses by the oral, parenteral or topical routes. Administration of the active compound may range from continuous (intravenous drip) to several oral administrations per day (for example, Q.O.D. or Q.I.D.) and may include oral, topical, parenteral, intramuscular, intravenous, sub-cutaneous, transdermal (which may include a penetration enhancement agent), buccal, sublingual and suppository administration, among other routes of administration. Enteric coated oral tablets may also be used to enhance bioavailability of the compounds from an oral route of administration. The most effective dosage form will depend upon the pharmacokinetics of the particular agent(s) chosen as well as the severity of disease in the patient. Administration of compounds according to the present disclosure as sprays, mists, or aerosols for intra-nasal, intra-tracheal or pulmonary administration may also be used. The present disclosure therefore also is directed to pharmaceutical compositions comprising an effective amount of compound as described herein, optionally in combination with a pharmaceutically acceptable carrier, additive or excipient. Compounds according to the present disclosure may be administered in immediate release, intermediate release or sustained or controlled release forms. In some embodiments, sustained or controlled release forms are y administered orally, but also in suppository and transdermal or other topical forms. Intramuscular injections in liposomal form may also be used to control or sustain the release of compound at an injection site.

In some embodiments, the pharmaceutical compositions as described herein is administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously.

In some embodiments, sterile injectable forms of the compositions as described herein are aqueous or oleaginous suspension. These suspensions may be formulated using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil, castor oil or soybean oil, especially in their polyoxyethylated versions. These oil solutions or suspensions may also contain a long-chain alcohol diluent or dispersant, such as Ph. Helv or similar alcohol.

In some embodiments, the pharmaceutical compositions as described herein are orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are used orally, the active ingredient is combined with emulsifying and suspending agents. If desired, certain sweetening, flavoring or coloring agents may also be added.

In some embodiments, the pharmaceutical compositions as described herein are administered in the form of suppositories for rectal administration. These can be prepared by mixing the agent with a suitable non-irritating excipient, which is solid at room temperature but liquid at rectal temperature and therefore will melt in the rectum to release the drug. Such materials include cocoa butter, beeswax and polyethylene glycols.

In some embodiments, the pharmaceutical compositions as described herein are administered topically. Suitable topical formulations are readily prepared for each of these areas or organs. Topical application for the lower intestinal tract can be effected in a rectal suppository formulation (see above) or in a suitable enema formulation. Topically-acceptable transdermal patches may also be used.

In some embodiments, for topical applications, the pharmaceutical compositions are formulated in a suitable ointment containing the active component suspended or dissolved in one or more carriers. Carriers for topical administration of the compounds of this disclosure include, but are not limited to, mineral oil, liquid petrolatum, DMSO, white petrolatum, propylene glycol, polyoxyethylene, polyoxypropylene compound, emulsifying wax and water. In some embodiments, the compounds may be coated onto a stent which is to be surgically implanted into a patient in order to inhibit or reduce the likelihood of occlusion occurring in the stent in the patient.

In some embodiments, the pharmaceutical compositions are formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

In some embodiments, for ophthalmic use, the pharmaceutical compositions are formulated as micronized suspensions in isotonic, pH adjusted sterile saline, or as solutions in isotonic, pH adjusted sterile saline, either with or without a preservative such as benzylalkonium chloride. Alternatively, for ophthalmic uses, the pharmaceutical compositions may be formulated in an ointment such as petrolatum.

In some embodiments, the pharmaceutical compositions as described herein are administered by nasal aerosol or inhalation. Such compositions are prepared according to techniques described herein relating to pharmaceutical compositions and may be prepared as solutions in saline, employing benzyl alcohol or other suitable preservatives, absorption promoters to enhance bioavailability, fluorocarbons, and/or other conventional solubilizing or dispersing agents. In some embodiments, the description provides formulations comprising liposomes including an effective amount of at least one of an ETBR antagonist or a caspase-8 inhibitor or a combination thereof, and/or an effective amount of at least one of an ETAR antagonist, an anti-PD1 antibody, a bRAF inhibitor, niacinamide or a combination thereof, wherein the liposome formulation is configured or adapted for intranasal delivery or sublingual delivery. In a further embodiment, the liposomes further comprise an additional anti-cancer agent as described above.

In some embodiments, the compositions should be formulated to contain between about 0.05 milligram to about 750 milligrams or more, for example about 1 milligram to about 600 milligrams, or about 10 milligrams to about 500 milligrams of active ingredient, alone or in combination with at least one other compound according to the present disclosure. It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

In some embodiments, a patient or subject in need of therapy using compounds according to the methods described herein is treated by administering to the patient (subject) an effective amount of the compound according to the present disclosure including pharmaceutically acceptable salts, solvates or polymorphs thereof, optionally in a pharmaceutically acceptable carrier or diluent, either alone, or in combination with other known erythopoiesis stimulating agents as otherwise identified herein.

In some embodiments, the compounds or compositions herein are administered orally, parenterally, intradermally, by an injection (intravenously, subcutaneously, or intramuscularly), topically, including transdermally, in liquid, cream, gel, or solid form, or by aerosol form.

In some embodiments, the active ingredients are included in the pharmaceutically acceptable carrier or diluent in an amount sufficient to deliver to a patient a therapeutically effective amount for the desired indication, without causing serious toxic effects in the patient treated. An exemplary dose of the active compound for all of the herein-mentioned conditions is in the range from about 10 ng/kg to 300 ng/kg, about 10 ng/kg to 1 μg/kg, about 1 μg/kg to 10 μg/kg, about 10 μg/kg to 100 μg/kg, about 100 μg/kg to 1000 μg/kg, about 1 mg/kg to 30 mg/kg, about 1 mg/kg to 300 mg/kg, or 0.1 to 100 mg/kg per day, more generally 0.5 to about 25 mg per kilogram body weight of the recipient/patient per day. A typical topical dosage will range from 0.01-5% wt/wt in a suitable carrier.

In some embodiments, the active ingredient herein is conveniently administered in any suitable unit dosage form, including but not limited to, one containing less than 1 mg, 1 mg to 3000 mg, for example 5 to 500 mg of active ingredient per unit dosage form. An oral dosage of about 25-250 mg is often convenient.

In some embodiments, the active ingredient is administered to achieve peak plasma concentrations of the active compound of about 0.00001-30 mM, for example about 0.1-30 μM. This may be achieved, for example, by the intravenous injection of a solution or formulation of the active ingredient, optionally in saline, or an aqueous medium or administered as a bolus of the active ingredient. Oral administration is also appropriate to generate effective plasma concentrations of active agent.

Methods for Treatment

Disclosed herein are methods for treating or ameliorating a disease, disorder or symptom thereof in a subject or a patient, e.g., an animal such as a human, comprising administering to a subject in need thereof an effective amount, e.g., a therapeutically effective amount of a pharmaceutical composition comprising a compound of formula (4), formula (5), formula (6), formula (7), formula (8) or formula (9), wherein the composition is effective for treating or ameliorating the disease or disorder or symptom thereof in the subject. In some embodiments, the disease or disorder is an ETBR-related cancer or a cancer that is insensitive to immune based therapy or both. In some embodiments, the ETBR-related cancer is at least one of breast cancer, metastatic breast cancer, melanoma, squamous cell carcinoma, glioblastoma or a combination thereof. In some embodiments, the cancer is a solid tumor cancer. In some embodiments, the ETBR-related cancer to be treated does not include breast cancer, melanoma, metastatic breast cancer or metastatic melanoma.

In some embodiments, the administration of an ETBR antagonist of formula (4), formula (5), formula (6), formula (7), formula (8) or formula (9) alone or in a combination with administration of at least one ETBR antagonist and an immune checkpoint inhibitor is sufficient to effectuate the treatment or amelioration of at least one symptom of cancer. In some embodiments, administration of the ETBR antagonist of formula (4), formula (5), formula (6), formula (7), formula (8) or formula (9) alone or in a combination with immune checkpoint inhibitor effectuates stimulation or enhancement of tumor infiltrating lymphocytes, macrophages, tertiary lymphoid organ formation or a combination thereof. In some embodiments, treatment or amelioration of cancer or stimulation or enhancement of tumor infiltrating lymphocytes, macrophages, induce tertiary lymphoid organ formation or a combination thereof, as determined using a V600E+ SM1 cancer model in mice, e.g., C57BL/6 mouse model. In some embodiments, the at least one ETBR antagonist and immune checkpoint inhibitor (whether in single formulation or separate) are administered in unit dosage forms. In some embodiments, the unit dosage form or forms comprises a therapeutically effective amount of each of the at least one ETBR antagonist, and the immune checkpoint inhibitor.

In some embodiments, the description provides methods for treating cancer in a subject, e.g., a solid tumor cancer, comprising administering to a subject in need thereof an effective dose of an ETBR antagonist of formula (4), formula (5), formula (6), formula (7), formula (8) or formula (9) alone or in a combination with an immune checkpoint inhibitor, wherein the administering effectuates the treatment or amelioration of at least one symptom of the cancer.

In some embodiments, the description provides methods of treating cancer in a subject comprising administering to a subject in need thereof an effective dose of an ETBR antagonist of formula (4), formula (5), formula (6), formula (7), formula (8) or formula (9), and administering to the subject an immune checkpoint inhibitor, wherein the administrations effectuate at least one of:

a. enhancement or stimulation of tumor infiltrating lymphocytes (TILs),

b. increased tumor associated macrophages (TAMs),

c. enhancement or stimulation of tertiary lymphoid organ (TLO) formation or

d. a combination thereof, and

thereby treating or ameliorating at least one symptom of the cancer. In some embodiments, (a)-(d) are determined in a human by biopsy or in an animal model. In some embodiments, the animal model is a V600E+ SM1 cancer model in mice, e.g., C57BL/6 mouse model.

In some embodiments, a method for treating cancer herein comprises administering to a patient in need thereof at least one ETBR antagonist of formula (4), formula (5), formula (6), formula (7), formula (8) or formula (9), wherein the at least one ETBR antagonist is effective in treating or ameliorating at least one symptom of the cancer in the patient. In some embodiments, the cancer is an ETBR-related cancer, e.g., an ETBR-related solid tumor cancer. In some embodiments, the ETBR-related cancer is at least one of breast cancer, melanoma, squamous cell carcinoma, glioblastoma, ovarian cancer, pancreatic cancer or a combination thereof. In some embodiments, the cancer is a solid tumor cancer. In further embodiments, the cancer is not breast cancer, melanoma, metastatic breast cancer or metastatic melanoma.

In some embodiments, the method comprises administering a composition comprising an effective amount of at least one ETBR antagonist of formula (4), formula (5), formula (6), formula (7), formula (8) or formula (9), and a pharmaceutically acceptable carrier or excipient for example dimethyl sulfoxide (DMSO), LYOCELL (reversed cubic phase liquid crystal dispersion), soybean oil, INTRAVAIL (transmucosal absorption enhancement agents), PROTEK (protein stabilization excipients), or hydrogel, or any combination thereof. In some embodiments, the composition is administered in unit dosage form.

In some embodiments, the method further comprises administering an additional anti-oncologic agent in combination with, e.g., either in the same or separate formulations, an ETBR antagonist described herein. In some embodiments, the anti-oncologic agent is an anti-PD1 antibody or anti-PD-L1 antibody. In some embodiments, the anti-oncologic agent, e.g., anti-PD1 or anti-PD-L1 antibody is administered as a composition comprising a pharmaceutically acceptable carrier or excipient.

In some embodiments, the method comprises administering a combination comprising at least one ETBR antagonist of formula (4), formula (5), formula (6), formula (7), formula (8) or formula (9), and at least one additional anti-oncologic agent as described herein. In some embodiments, the combination comprises a pharmaceutically acceptable carrier or excipient. In some embodiments, the combination comprises an amount of an immune checkpoint inhibitor and a therapeutically effective amount of the at least one ETBR antagonist. In some embodiments, the immune checkpoint inhibitor is an anti-PD1 antibody.

In some embodiments, the pharmaceutical compositions are delivered intravenously, intramuscularly, subcutaneously, orally, intranasally, sublingually, transdermally, topically, intraperitoneally, parenterally, intranasally, or intracranially.

In some embodiments, a method for treating ETBR-related metastatic brain cancer is provided. The method comprises administering an effective amount to a subject in need thereof a pharmaceutical composition of the present disclosure, wherein the pharmaceutical composition is effective for treating or ameliorating a symptom of ETBR-related metastatic brain cancer. In some embodiments, the ETBR-related metastatic brain cancer is metastatic melanoma-related brain cancer, metastatic squamous cell carcinoma-related brain cancer, glioblastoma or a combination thereof. In some embodiments, the composition comprises an effective amount of an ETBR antagonist of formula (4), formula (5), formula (6), formula (7), formula (8) or formula (9), and a pharmaceutically acceptable carrier.

In some embodiments, the description provides methods for treating a solid tumor cancer in a human subject, comprising administering effective doses of an ETBR antagonist of formula (4), formula (5), formula (6), formula (7), formula (8) or formula (9) and further administering an immune checkpoint inhibitor to the subject in need thereof, wherein the administration of the ETBR antagonist and immune checkpoint inhibitor effectuates at least one of: (i) enhancement or stimulation of tumor infiltrating lymphocytes (TILs), (ii) increased tumor associated macrophages (TAMs), (iii) enhancement or stimulation of tertiary lymphoid organ (TLO) formation or (iv) a combination thereof, wherein the ETBR antagonist and immune checkpoint inhibitor effectuate the treatment or alleviation of at least one symptom of the solid tumor cancer. In some embodiments, the formation of (i)-(iv) is performed in a mouse model. In some embodiments, the mouse model is the V600E+ SM1 cancer model in C57BL/6 mice. In some embodiments, the immune checkpoint inhibitor is an anti-PD1 or anti-PD-L1 antibody. In some embodiments, the ETBR antagonist and immune checkpoint inhibitor are administered separately. In some embodiments, the ETBR antagonist and immune checkpoint inhibitor are administered in the same formulation.

In some embodiments, the description provides a method of inhibiting melanoma invasion and metastasis in a subject in need thereof comprising administering to the subject in need thereof an effective amount, e.g., a therapeutically effective amount of a compound of formula (4), formula (5), formula (6), formula (7), formula (8) or formula (9), wherein the composition is effective for inhibiting melanoma invasion and metastasis.

In some embodiments, the description provides a method of inducing melanoma cell death (apoptosis) comprising administering to a subject in need thereof an effective amount, of a compound of formula (4), formula (5), formula (6), formula (7), formula (8) or formula (9), wherein the composition is effective for inducing melanoma cell death.

In some embodiments, the description provides a method of inhibiting blood supply to melanoma tumors in a patient comprising administering to a subject in need thereof an effective amount of a compound of formula (4), formula (5), formula (6), formula (7), formula (8) or formula (9), wherein the composition is effective for inhibiting blood supply to melanoma tumors.

In some embodiments, the pharmaceutical composition comprises about 1% to about 95% of the active ingredient, single-dose forms of administration comprising about 20% to about 90% of the active ingredient and administration forms which are not single-dose comprising about 5% to about 20% of the active ingredient. Unit dose forms are, for example, coated tablets, tablets, ampoules, vials, suppositories or capsules. Other forms of administration are, for example, ointments, creams, pastes, foams, tinctures, lipsticks, drops, sprays, dispersions and the like. Examples are capsules containing from about 0.05 g to about 1.0 g of the active ingredient.

In some embodiments, the active ingredient is included in the pharmaceutically acceptable carrier or diluent in an amount sufficient to deliver to a patient a therapeutically effective amount for the desired indication, without causing serious toxic effects in the patient treated. An exemplary dose of the active compound for all of the herein-mentioned conditions is in the range from about 10 ng/kg to 300 mg/kg, for example 0.1 to 100 mg/kg per day, more generally 0.5 to about 25 mg per kilogram body weight of the recipient/patient per day. A typical topical dosage will range from 0.01-5% wt/wt in a suitable carrier. The compound is conveniently administered in any suitable unit dosage form, including but not limited to one containing less than 1 mg, 1 mg to 3000 mg, for example 5 to 500 mg of active ingredient per unit dosage form. An oral dosage of about 25-250 mg is often convenient. In some embodiments, the active ingredient is administered to achieve peak plasma concentrations of the active compound of about 0.00001-30 mM, for example about 0.1-30 μM.

Dosage Regimen

Disclosed herein is a treatment regimen. In some embodiments, the treatment regimen includes a dosage pharmaceutical composition with about 100 μg to about 4000 μg of each included active ingredient (i.e., at least one ETBR antagonist of a compound of formula (4), formula (5), formula (6), formula (7), formula (8) or formula (9), the ETAR antagonist, the anti-PD1 antibody, the bRAF inhibitor, the niacinamide, or the caspase-8 inhibitor). The dosage can be a sustained release dosage in which about 50 μg to about 3000 μg of each of the active ingredients is an initial burst, while about 50 μg to about 3000 μg of the each of the active ingredients is a sustained release over 2 hours.

In some embodiments, the compound of formula (4), formula (5), formula (6), formula (7), formula (8) or formula (9) can be present in any of the dosage formulation (e.g., initial burst, sustained release dosage, etc.) in about 100 μg to about 4000 μg, about 100 μg to about 3750 μg, about 100 μg to about 3500 μg, about 100 μg to about 3250 μg, about 100 μg to about 3000 μg, about 100 μg to about 2750 μg, about 100 μg to about 2500 μg, about 100 μg to about 2250 μg, about 100 μg to about 2000 μg, about 100 μg to about 1750 μg, about 100 μg to about 1500 μg, about 100 μg to about 1250 μg, about 100 μg to about 1000 μg, about 100 μg to about 750 μg, about 100 μg to about 500 μg, about 250 μg to about 4000 μg, about 250 μg to about 3750 μg, about 250 μg to about 3500 μg, about 250 μg to about 3250 μg, about 250 μg to about 3000 μg, about 250 μg to about 2750 μg, about 250 μg to about 2500 μg, about 250 μg to about 2250 μg, about 250 μg to about 2000 μg, about 250 μg to about 1750 μg, about 250 μg to about 1500 μg, about 250 μg to about 1250 μg, about 250 μg to about 1000 μg, about 250 μg to about 750 μg, about 250 μg to about 500 μg, about 500 μg to about 4000 μg, about 500 μg to about 3750 μg, about 500 μg to about 3500 μg, about 500 μg to about 3250 μg, about 500 μg to about 3000 μg, about 500 μg to about 2750 μg, about 500 μg to about 2500 μg, about 500 μg to about 2250 μg, about 500 μg to about 2000 μg, about 500 μg to about 1750 μg, about 500 μg to about 1500 μg, about 500 μg to about 1250 μg, about 500 μg to about 1000 μg, about 500 μg to about 750 μg, about 750 μg to about 4000 μg, about 750 μg to about 3750 μg, about 750 μg to about 3500 μg, about 750 μg to about 3250 μg, about 750 μg to about 3000 μg, about 750 μg to about 2750 μg, about 750 μg to about 2500 μg, about 750 μg to about 2250 μg, about 750 μg to about 2000 μg, about 750 μg to about 1750 μg, about 750 μg to about 1500 μg, about 750 μg to about 1250 μg, about 750 μg to about 1000 μg, about 1000 μg to about 4000 μg, about 1000 μg to about 3750 μg, about 1000 μg to about 3500 μg, about 1000 μg to about 3250 μg, about 1000 μg to about 3000 μg, about 1000 μg to about 2750 μg, about 1000 μg to about 2500 μg, about 1000 μg to about 2250 μg, about 1000 μg to about 2000 μg, about 1000 μg to about 1750 μg, about 1000 μg to about 1500 μg, about 1000 μg to about 1250 μg, about 1250 μg to about 4000 μg, about 1250 μg to about 3750 μg, about 1250 μg to about 3500 μg, about 1250 μg to about 3250 μg, about 1250 μg to about 3000 μg, about 1250 μg to about 2750 μg, about 1250 μg to about 2500 μg, about 1250 μg to about 2250 μg, about 1250 μg to about 2000 μg, about 1250 μg to about 1750 μg, about 1250 μg to about 1500 μg, about 1500 μg to about 4000 μg, about 1500 μg to about 3750 μg, about 1500 μg to about 3500 μg, about 1500 μg to about 3250 μg, about 1500 μg to about 3000 μg, about 1500 μg to about 2750 μg, about 1500 μg to about 2500 μg, about 1500 μg to about 2250 μg, about 1500 μg to about 2000 μg, about 1500 μg to about 1750 μg, about 1750 μg to about 4000 μg, about 1750 μg to about 3750 μg, about 1750 μg to about 3500 μg, about 1750 μg to about 3250 μg, about 1750 μg to about 3000 μg, about 1750 μg to about 2750 μg, about 1750 μg to about 2500 μg, about 1750 μg to about 2250 μg, about 1750 μg to about 2000 μg, about 2000 μg to about 4000 μg, about 2000 μg to about 3750 μg, about 2000 μg to about 3500 μg, about 2000 μg to about 3250 μg, about 2000 μg to about 3000 μg, about 2000 μg to about 2750 μg, about 2000 μg to about 2500 μg, about 2000 μg to about 2250 μg, about 2250 μg to about 4000 μg, about 2250 μg to about 3750 μg, about 2250 μg to about 3500 μg, about 2250 μg to about 3250 μg, about 2250 μg to about 3000 μg, about 2250 μg to about 2750 μg, about 2250 μg to about 2500 μg, about 2500 μg to about 4000 μg, about 2500 μg to about 3750 μg, about 2500 μg to about 3500 μg, about 2500 μg to about 3250 μg, about 2500 μg to about 3000 μg, about 2500 μg to about 2750 μg, about 2750 μg to about 4000 μg, about 2750 μg to about 3750 μg, about 2750 μg to about 3500 μg, about 2750 μg to about 3250 μg, about 2750 μg to about 3000 μg, about 3000 μg to about 4000 μg, about 3000 μg to about 3750 μg, about 3000 μg to about 3500 μg, about 3000 μg to about 3250 μg, about 3250 μg to about 4000 μg, about 3250 μg to about 3750 μg, about 3250 μg to about 3500 μg, about 3500 μg to about 4000 μg, about 3500 μg to about 3750 μg, or about 3750 μg to about 4000 μg.

In some embodiments, each active ingredient of a pharmaceutical composition of the present disclosure is present in about 0.1 mg/mL to about 50 mg/mL, about 0.1 mg/mL to about 25 mg/mL, about 0.1 mg/mL to about 10 mg/mL, about 1 mg/mL to about 50 mg/mL, about 1 mg/mL to about 25 mg/mL, about 1 mg/mL to about 10 mg/mL, about 0.1 mg/mL to about 5.0 mg/mL (e.g., about 0.1 mg/mL to about 4.5 mg/mL, about 0.1 mg/mL to about 4.0 mg/mL, about 0.1 mg/mL to about 3.5 mg/mL, about 0.1 mg/mL to about 3.0 mg/mL, about 0.1 mg/mL to about 2.5 mg/mL, about 0.1 mg/mL to about 2.0 mg/mL, about 0.1 mg/mL to about 1.5 mg/mL, about 0.1 mg/mL to about 1.0 mg/mL, about 0.1 mg/mL to about 0.5 mg/mL, about 0.5 mg/mL to about 4.5 mg/mL, about 0.5 mg/mL to about 4.0 mg/mL, about 0.5 mg/mL to about 3.5 mg/mL, about 0.5 mg/mL to about 3.0 mg/mL, about 0.5 mg/mL to about 2.5 mg/mL, about 0.5 mg/mL to about 2.0 mg/mL, about 0.5 mg/mL to about 1.5 mg/mL, about 0.5 mg/mL to about 1.0 mg/mL, about 1.0 mg/mL to about 4.5 mg/mL, about 1.0 mg/mL to about 4.0 mg/mL, about 1.0 mg/mL to about 3.5 mg/mL, about 1.0 mg/mL to about 3.0 mg/mL, about 1.0 mg/mL to about 2.5 mg/mL, about 1.0 mg/mL to about 2.0 mg/mL, about 1.0 mg/mL to about 1.5 mg/mL, about 1.5 mg/mL to about 4.5 mg/mL, about 1.5 mg/mL to about 4.0 mg/mL, about 1.5 mg/mL to about 3.5 mg/mL, about 1.5 mg/mL to about 3.0 mg/mL, about 1.5 mg/mL to about 2.5 mg/mL, about 1.5 mg/mL to about 2.0 mg/mL, about 2.0 mg/mL to about 4.5 mg/mL, about 2.0 mg/mL to about 4.0 mg/mL, about 2.0 mg/mL to about 3.5 mg/mL, about 2.0 mg/mL to about 3.0 mg/mL, about 2.0 mg/mL to about 2.5 mg/mL, about 2.5 mg/mL to about 4.5 mg/mL, about 2.5 mg/mL to about 4.0 mg/mL, about 2.5 mg/mL to about 3.5 mg/mL, about 2.5 mg/mL to about 3.0 mg/mL, about 3.0 mg/mL to about 4.5 mg/mL, about 3.0 mg/mL to about 4.0 mg/mL, about 3.0 mg/mL to about 3.5 mg/mL, about 3.5 mg/mL to about 4.5 mg/mL, about 3.5 mg/mL to about 4.0 mg/mL, or about 3.5 mg/mL to about 4.5 mg/mL, relative to the pharmaceutical composition).

In some embodiments, each active ingredient of a pharmaceutical composition of the present disclosure is present in about 0.1 μg/mL to about 50 μg/mL, about 0.1 μg/mL to about 25 μg/mL, about 0.1 μg/mL to about 10 μg/mL, about 1 μg/mL to about 50 μg/mL, about 1 μg/mL to about 25 μg/mL, about 1 μg/mL to about 10 μg/mL, about 0.1 μg/mL to about 5.0 μg/mL, e.g., about 1 μg/mL to about 5 μg/mL, about 0.1 μg/mL to about 4.0 μg/mL, about 0.1 μg/mL to about 3.5 μg/mL, about 0.1 μg/mL to about 3.0 μg/mL, about 0.1 μg/mL to about 2.5 μg/mL, about 0.1 μg/mL to about 2.0 μg/mL, about 0.1 μg/mL to about 1.5 μg/mL, about 0.1 μg/mL to about 1.0 μg/mL, about 0.1 μg/mL to about 0.5 μg/mL, about 0.5 μg/mL to about 4.5 μg/mL, about 0.5 μg/mL to about 4.0 μg/mL, about 0.5 μg/mL to about 3.5 μg/mL, about 0.5 μg/mL to about 3.0 μg/mL, about 0.5 μg/mL to about 2.5 μg/mL, about 0.5 μg/mL to about 2.0 μg/mL, about 0.5 μg/mL to about 1.5 μg/mL, about 0.5 μg/mL to about 1.0 μg/mL, about 1.0 μg/mL to about 4.5 μg/mL, about 1.0 μg/mL to about 4.0 μg/mL, about 1.0 μg/mL to about 3.5 μg/mL, about 1.0 μg/mL to about 3.0 μg/mL, about 1.0 μg/mL to about 2.5 μg/mL, about 1.0 μg/mL to about 2.0 μg/mL, about 1.0 μg/mL to about 1.5 μg/mL, about 1.5 μg/mL to about 4.5 μg/mL, about 1.5 μg/mL to about 4.0 μg/mL, about 1.5 μg/mL to about 3.5 μg/mL, about 1.5 μg/mL to about 3.0 μg/mL, about 1.5 μg/mL to about 2.5 μg/mL, about 1.5 μg/mL to about 2.0 μg/mL, about 2.0 μg/mL to about 4.5 μg/mL, about 2.0 μg/mL to about 4.0 μg/mL, about 2.0 μg/mL to about 3.5 μg/mL, about 2.0 μg/mL to about 3.0 μg/mL, about 2.0 μg/mL to about 2.5 μg/mL, about 2.5 μg/mL to about 4.5 μg/mL, about 2.5 μg/mL to about 4.0 μg/mL, about 2.5 μg/mL to about 3.5 μg/mL, about 2.5 μg/mL to about 3.0 μg/mL, about 3.0 μg/mL to about 4.5 μg/mL, about 3.0 μg/mL to about 4.0 μg/mL, about 3.0 μg/mL to about 3.5 μg/mL, about 3.5 μg/mL to about 4.5 μg/mL, about 3.5 μg/mL to about 4.0 μg/mL, or about 3.5 μg/mL to about 4.5 μg/mL, relative to the pharmaceutical composition.

### EXAMPLES

Unless otherwise noted, reagents and solvents are used as received from commercial suppliers. Anhydrous solvents and oven-dried glassware are used for synthetic transformations sensitive to moisture and/or oxygen. Yields are not optimized. Reaction times are approximate and are not optimized. Column chromatography and thin layer chromatography (TLC) are performed on silica gel unless otherwise noted.

In some embodiments, ETBR Antagonist compounds disclosed herein are synthesized according to the following examples.

General Scheme 1 for the Synthesis of ETBR Antagonists

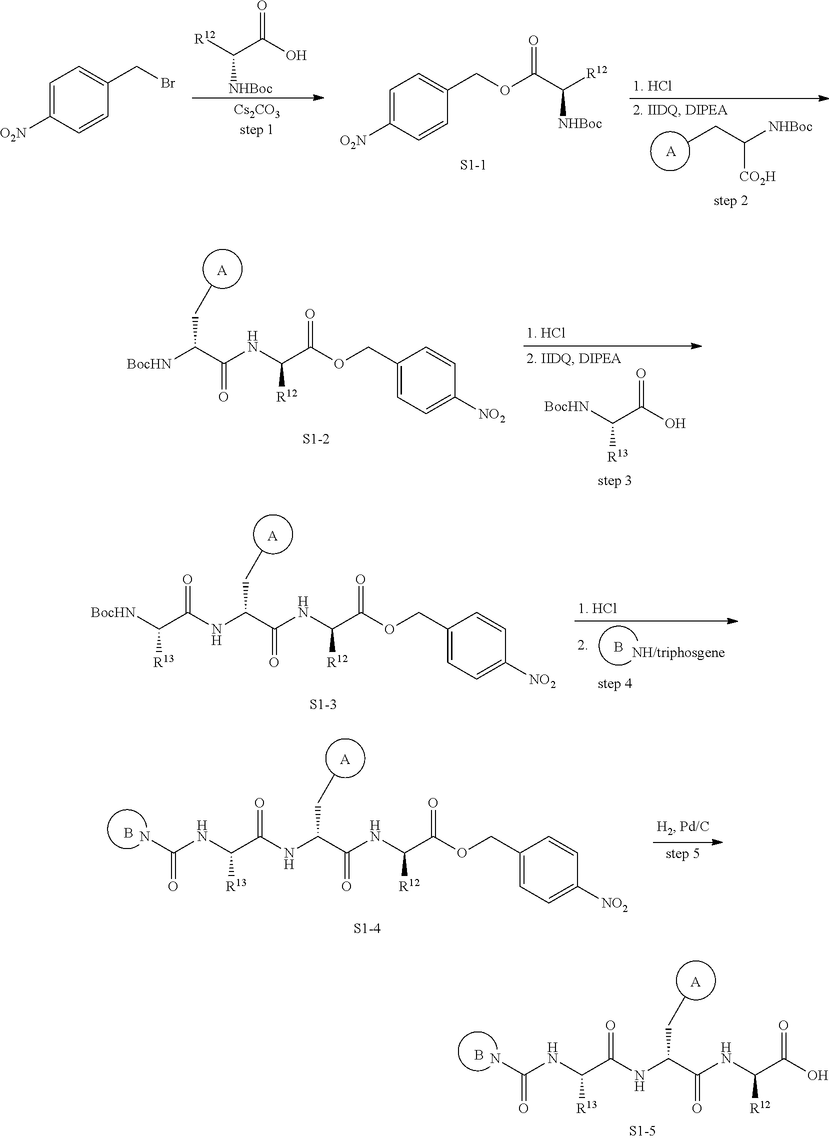

wherein
R¹² is C₁-C₆ alkyl or C₁-C₆ heteroalkyl;
R¹³ is C₁-C₆ alkyl or C₁-C₆ heteroalkyl;
ring A is aryl; and
ring B is C₂-C₈ heterocycloalkyl.

General Scheme 2 for the Synthesis of ETBR Antagonists—BQ-788 Analogues

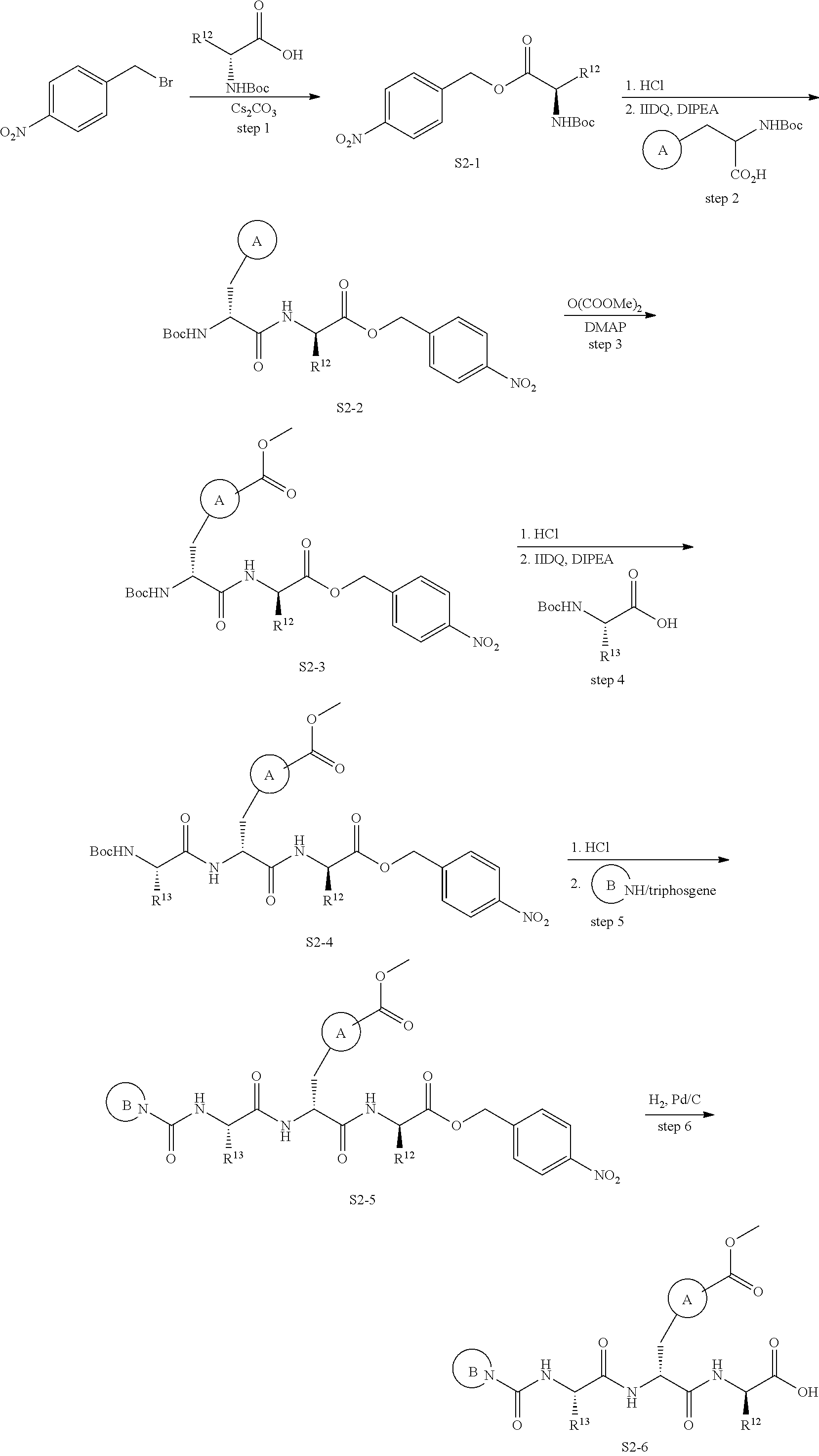

wherein
R¹² is C₁-C₆ alkyl or C₁-C₆ heteroalkyl;
R¹³ is C₁-C₆ alkyl or C₁-C₆ heteroalkyl;
ring A is aryl; and
ring B is C₂-C₈ heterocycloalkyl.

General Scheme 3 for the Synthesis of Tetracyclic ETBR Antagonists

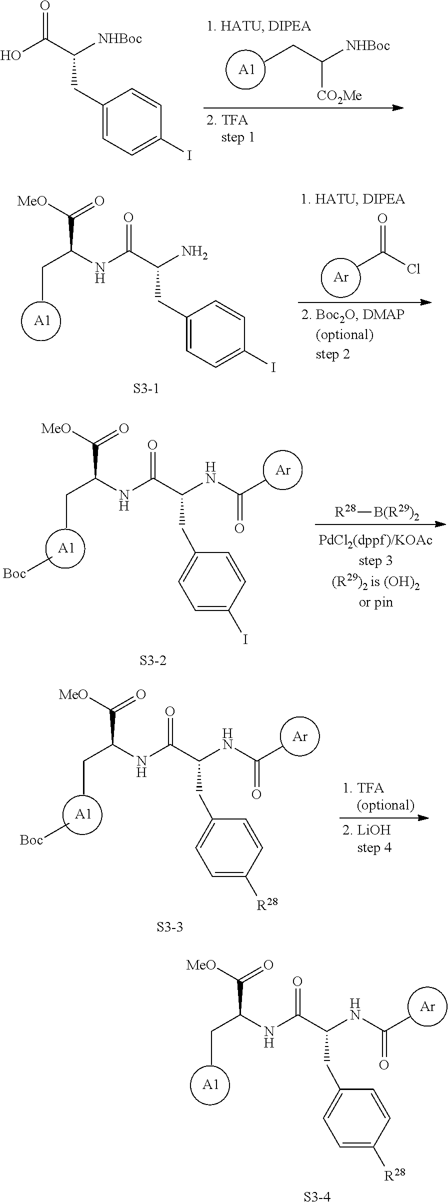

wherein
ring A1 is substituted or unsubstituted monocyclic or bicyclic heteroaryl
ring Ar is phenyl substituted with 1, 2, or 3 substituents selected from halogen, cyano, C₁-C₆ alkyl, C₃-C₈ cycloalkyl, or C₁-C₆ alkoxy; and
R²⁸ is phenyl, substituted or unsubstituted 5-membered heteroaryl, or substituted or unsubstituted 6-membered heteroaryl.

General Scheme 4 for the Synthesis of Pyrrolidine ETBR Antagonists

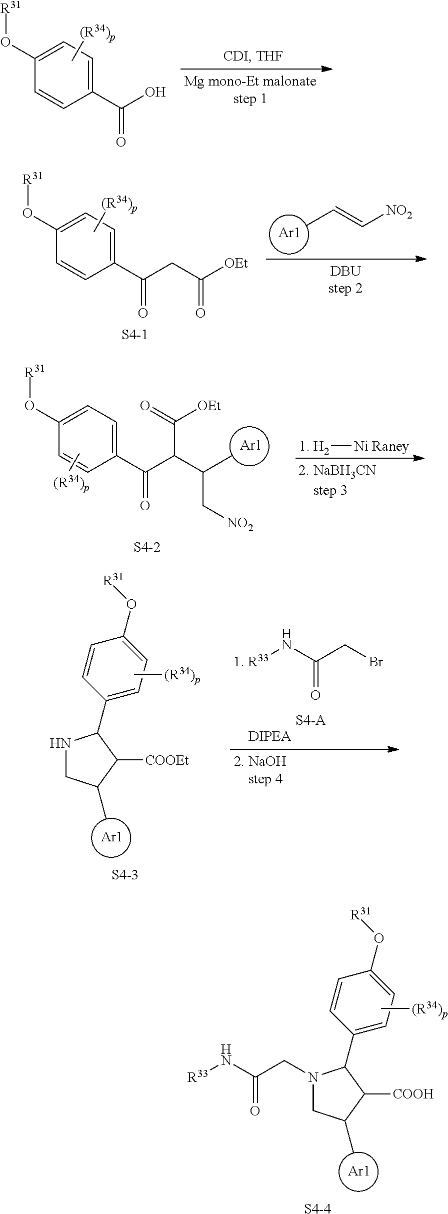

wherein
ring Ar1 is substituted or unsubstituted phenyl;
R³¹ is substituted or unsubstituted C₁-C₆ alkyl;
R³³ is substituted or unsubstituted aryl, substituted or unsubstituted heteroaryl, or —CH(R³³)₂,
wherein each R³⁵ is substituted or unsubstituted aryl;
each R³⁴ is independently deuterium, halogen, cyano, C₁-C₆ alkyl, C₁-C₆ haloalkyl, and C₁-C₆ alkoxy; and
p is an integer from 0-2.

General Scheme 5 for the Synthesis of Pyrimidine ETBR Antagonists

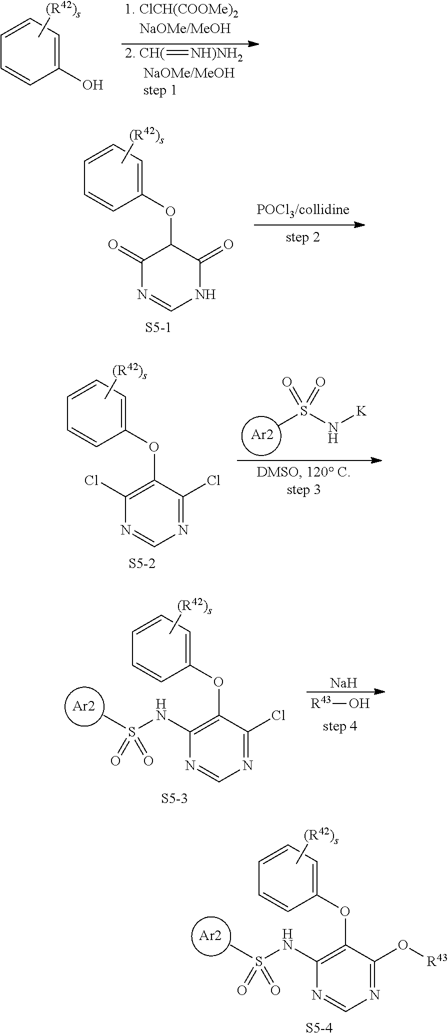

wherein
ring Ar2 is substituted or unsubstituted phenyl;
each R⁴² is independently deuterium, halogen, cyano, C₁-C₆ alkyl, C₁-C₆ haloalkyl, and C₁-C₆ alkoxy;
R⁴³ is substituted C₁-C₆ alkyl substituted or unsubstituted C₁-C₆ alkyl, substituted C₃-C₈ cycloalkyl, or substituted or unsubstituted C₂-C₇ heterocycloalkyl; and
s is an integer from 0-2.

General Scheme 6 for the Synthesis of Heteroaryl ETBR Antagonists

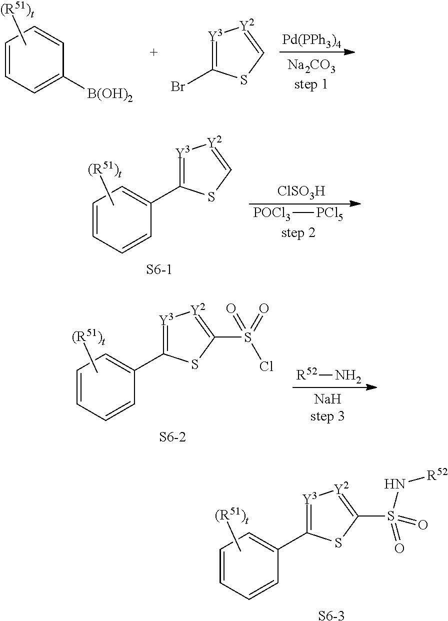

wherein
R⁵¹ is halogen, cyano, C₁-C₆ alkyl, C₁-C₆ haloalkyl, or C₁-C₆ alkoxy;
each Y² and Y³ is independently N or CH;
R⁵² is substituted or unsubstituted 5-membered heteroaryl or substituted or unsubstituted 6-membered heteroaryl; wherein if R⁵² is substituted then it is substituted with 1, 2, or 3 substituents selected from halogen, cyano, C₁-C₆ alkyl, C₁-C₆ haloalkyl, and C₁-C₆ alkoxy; and
t is an integer from 0-3.

### Example 1. Synthesis and Characterization of ETBR Antagonists

### Step 1:

Cs₂CO₃ (5.86 g; 18.00 mmol) and p-nitrobenzyl bromide (2.04 g; 9.45 mmol) are added to a solution of R¹²CH(NHBoc)COOH (9.00 mmol) in DMF (10 mL). The reaction is stirred for 2.5 h at room temperature and EtOAc (100 mL) is added. The solution is then washed with saturated NaHCO₃ (2×50 mL). Aqueous layers are combined and extracted with EtOAc (2×50 mL). The organic layers are combined, are washed with brine, are dried with anhydrous MgSO₄, are filtered and concentrated under reduced pressure to give a yellow oily product. The crude material is purified using silica gel chromatography to yield compound 51-1.

### Step 2:

HCl (g) anhydrous is bubbled for 1 h in a flask containing ethyl ether (400 mL). Part of this solution (200 mL) is transferred into another flask containing compound S1-1 (8.55 mmol) and is stirred for 24 h. The reacting mixture is then concentrated under reduced pressure. Additional ethyl ether (3×100 mL) is added and evaporated under reduced pressure repeatedly to remove the excess HCl. DCM (2×100 mL) is added and evaporated under reduced pressure repeatedly. The resulting solid is used without further purification.

A solution of IIDQ (5.24 g; 17.25 mmol) in DCM (10 mL) is added to a suspension of A-CH₂—CH(NHBoc)COOH (8.62 mmol) in DCM (30 mL) and is allowed to stir for 15 min at room temperature. A solution of the crude product from the previous step (8.62 mmol) and DIPEA (3.75 mL; 21.57 mmol) in DCM (20 mL) is added to the reaction. The reacting mixture is stirred for 24 h at room temperature. Then, it is washed with 10% citric acid (3×50 mL). The aqueous layers are combined and extracted with DCM (2×50 mL). The organic layers are combined, are washed with saturated NaHCO₃ and brine, are dried with anhydrous MgSO₄, are filtered and concentrated under reduced pressure to give an orange oily product. The crude material is purified using silica gel chromatography to yield compound S1-2.

### Step 3:

HCl (g) anhydrous is bubbled for 1 h in a flask containing ethyl ether (400 mL). Part of this solution (200 mL) is transferred into another flask containing compound S1-2 (6.35 mmol) and is stirred for 24 h. The reacting mixture is then concentrated under reduced pressure. Additional ethyl ether (3×100 mL) is added and evaporated under reduced pressure repeatedly to remove the excess HCl. DCM (2×100 mL) is added and evaporated under reduced pressure repeatedly. The resulting solid is used without further purification.

A solution of IIDQ (3.83 g; 12.61 mmol) in DCM (10 mL) is added to a solution of R¹³CH(NHBoc)COOH (6.30 mmol) in DCM (30 mL) and is allowed to stir for 15 min at room temperature. A solution of the crude product from the previous step (6.30 mmol) and DIPEA (2.3 mL; 15.70 mmol) in DCM (20 mL) is added to the reaction. The reacting mixture is allowed to stir for 24 h at room temperature. Then, it is washed with 10% citric acid (3×50 mL). The aqueous layers are combined and extracted with DCM (2×50 mL). The organic layers are combined and are washed with saturated NaHCO₃ and brine, are dried with anhydrous MgSO₄, are filtered and concentrated under reduced pressure to give an orange oily product. The crude material is purified using silica gel chromatography to yield compound S1-3.

### Step 4:

HCl(g) anhydrous is bubbled for 1 h in a flask containing ethyl ether (400 mL). Part of this solution (200 mL) is transferred into another flask containing compound S1-3 (4.60 mmol) and is stirred for 24 h. Then, the reacting mixture is concentrated under reduced pressure. Additional ethyl ether (3×100 mL) is added and evaporated under reduced pressure repeatedly to remove the excess HCl. DCM (2×100 mL) is added and evaporated under reduced pressure repeatedly. The resulting solid is used without further purification.

To a flame dried round bottom flask under nitrogen atmosphere, amine B (9.20 mmol) and DIPEA (2.00 mL; 11.50 mmol) are allowed to dissolve in DCM (15 mL) in an ice/NaCl bath at −15° C. Then, an immediately prepared solution of triphosgene (0.916 g; 3.08 mmol) in DCM (5 mL) is added over a 5 min period and is allowed to stir for another 15 min at −15° C. The bath is removed and the reaction is warmed up to room temperature for 1 h. The reacting mixture is then concentrated under reduced pressure and is dissolved in MeCN (20 mL) to yield amine B-carbonyl chloride. NaI (3.45 g; 23.00 mmol) is added and allowed to stir for 5 min. Subsequently, the crude product from the previous step (4.60 mmol) and DIPEA (2.00 mL; 11.50 mmol) are dissolved in MeCN (10 mL) and this solution is added to the flask under nitrogen atmosphere. The reacting mixture is allowed to stir for 24 h at room temperature. Reaction progress is monitored by TLC and fresh portions of amine B-carbonyl chloride are added to drive the reaction to completion if needed. The reaction mixture is then concentrated under reduced pressure, is diluted with DCM and is washed with 1N HCl (3×100 mL). Aqueous layers are combined and extracted with DCM (3×100 mL). The organic layers are combined and are washed with highly concentrated Na₂S₂O₅ and brine, are dried with anhydrous MgSO₄, are filtered and concentrated under reduced pressure to give a yellow pale solid product. The crude material is purified using silica gel chromatography to yield compound S1-4.

### Step 5:

5% Pd/C (catalytic amount ˜100 mg) is carefully added to a solution of compound S1-4 (2.40 mmol) in ethyl ether (15 mL) containing formic acid (0.300 mL; 2%). The solution is shaken under a hydrogen atmosphere (50 psi). After 2 h, the resulting mixture is filtered on Celite and is evaporated under reduced pressure to yield a yellow oily product. The crude material is purified using silica gel chromatography to yield compound S1-5.

### Example 2. Synthesis and Characterization of ETBR Antagonists—BQ-788 Analogues

### Step 1:

Cs₂CO₃ (5.86 g; 18.00 mmol) and p-nitrobenzyl bromide (2.04 g; 9.45 mmol) are added to a solution of R¹²CH(NHBoc)COOH (9.00 mmol) in DMF (10 mL). The reaction is stirred for 2.5 h at room temperature and EtOAc (100 mL) is added. The solution is then washed with saturated NaHCO₃ (2×50 mL). Aqueous layers are combined and extracted with EtOAc (2×50 mL). The organic layers are combined, are washed with brine, are dried with anhydrous MgSO₄, are filtered and concentrated under reduced pressure to give a yellow oily product. The crude material is purified using silica gel chromatography to yield compound S2-1.

### Step 2:

HCl (g) anhydrous is bubbled for 1 h in a flask containing ethyl ether (400 mL). Part of this solution (200 mL) is transferred into another flask containing compound S2-1 (8.55 mmol) and is stirred for 24 h. The reacting mixture is then concentrated under reduced pressure. Additional ethyl ether (3×100 mL) is added and evaporated under reduced pressure repeatedly to remove the excess HCl. DCM (2×100 mL) is added and evaporated under reduced pressure repeatedly. The resulting solid is used without further purification.

A solution of IIDQ (5.24 g; 17.25 mmol) in DCM (10 mL) is added to a suspension of A-CH₂—CH(NHBoc)COOH (8.62 mmol) in DCM (30 mL) and is allowed to stir for 15 min at room temperature. A solution of the crude product from the previous step (8.62 mmol) and DIPEA (3.75 mL; 21.57 mmol) in DCM (20 mL) is added to the reaction. The reacting mixture is stirred for 24 h at room temperature. Then, it is washed with 10% citric acid (3×50 mL). The aqueous layers are combined and extracted with DCM (2×50 mL). The organic layers are combined, are washed with saturated NaHCO₃ and brine, are dried with anhydrous MgSO₄, are filtered and concentrated under reduced pressure to give an orange oily product. The crude material is purified using silica gel chromatography to yield compound S2-2.

### Step 3:

DMAP (0.143 g; 1.16 mmol) followed by dimethyldicarbonate (5.21 g; 38.80 mmol) are added to a solution of compound S2-2 (7.77 mmol) in MeCN (125 mL). The resulting solution is allowed to stir for 24 h. Then, it is concentrated under reduced pressure. It is dissolved with EtOAc (100 mL) and is washed with 1N HCl (2×50 mL). The aqueous layers are combined and extracted with EtOAc (2×50 mL). The organic layers are combined and washed with water, are dried with anhydrous MgSO₄, are filtered and concentrated under reduced pressure to give a yellow pale solid product. The crude material is purified using silica gel chromatography to yield compound S2-3.

### Step 4:

HCl (g) anhydrous is bubbled for 1 h in a flask containing ethyl ether (400 mL). Part of this solution (200 mL) is transferred into another flask containing compound S2-3 (6.35 mmol) and is stirred for 24 h. The reacting mixture is then concentrated under reduced pressure. Additional ethyl ether (3×100 mL) is added and evaporated under reduced pressure repeatedly to remove the excess HCl. DCM (2×100 mL) is added and evaporated under reduced pressure repeatedly. The resulting solid is used without further purification.

A solution of IIDQ (3.83 g; 12.61 mmol) in DCM (10 mL) is added to a solution of R¹³CH(NHBoc)COOH (6.30 mmol) in DCM (30 mL) and is allowed to stir for 15 min at room temperature. A solution of the crude product from the previous step (6.30 mmol) and DIPEA (2.3 mL; 15.70 mmol) in DCM (20 mL) is added to the reaction. The reacting mixture is allowed to stir for 24 h at room temperature. Then, it is washed with 10% citric acid (3×50 mL). The aqueous layers are combined and extracted with DCM (2×50 mL). The organic layers are combined and are washed with saturated NaHCO₃ and brine, are dried with anhydrous MgSO₄, are filtered and concentrated under reduced pressure to give an orange oily product. The crude material is purified using silica gel chromatography to yield compound S2-4.

### Step 5:

HCl(g) anhydrous is bubbled for 1 h in a flask containing ethyl ether (400 mL). Part of this solution (200 mL) is transferred into another flask containing compound S2-4 (4.60 mmol) and is stirred for 24 h. Then, the reacting mixture is concentrated under reduced pressure. Additional ethyl ether (3×100 mL) is added and evaporated under reduced pressure repeatedly to remove the excess HCl. DCM (2×100 mL) is added and evaporated under reduced pressure repeatedly. The resulting solid is used without further purification.

To a flame dried round bottom flask under nitrogen atmosphere, amine B (9.20 mmol) and DIPEA (2.00 mL; 11.50 mmol) are allowed to dissolve in DCM (15 mL) in an ice/NaCl bath at −15° C. Then, an immediately prepared solution of triphosgene (0.916 g; 3.08 mmol) in DCM (5 mL) is added over a 5 min period and is allowed to stir for another 15 min at −15° C. The bath is removed and the reaction is warmed up to room temperature for 1 h. The reacting mixture is then concentrated under reduced pressure and is dissolved in MeCN (20 mL) to yield amine B-carbonyl chloride. NaI (3.45 g; 23.00 mmol) is added and allowed to stir for 5 min. Subsequently, the crude product from the previous step (4.60 mmol) and DIPEA (2.00 mL; 11.50 mmol) are dissolved in MeCN (10 mL) and this solution is added to the flask under nitrogen atmosphere. The reacting mixture is allowed to stir for 24 h at room temperature. Reaction progress is monitored by TLC and fresh portions of amine B-carbonyl chloride are added to drive the reaction to completion if needed. The reaction mixture is then concentrated under reduced pressure, is diluted with DCM and is washed with 1N HCl (3×100 mL). Aqueous layers are combined and extracted with DCM (3×100 mL). The organic layers are combined and are washed with highly concentrated Na₂S₂O₅ and brine, are dried with anhydrous MgSO₄, are filtered and concentrated under reduced pressure to give a yellow pale solid product. The crude material is purified using silica gel chromatography to yield compound S2-5.

### Step 6:

5% Pd/C (catalytic amount ˜100 mg) is carefully added to a solution of compound S2-5 (2.40 mmol) in ethyl ether (15 mL) containing formic acid (0.300 mL; 2%). The solution is shaken under a hydrogen atmosphere (50 psi). After 2 h, the resulting mixture is filtered on Celite and is evaporated under reduced pressure to yield a yellow oily product. The crude material is purified using silica gel chromatography to yield compound S2-6.

### Example 3. Synthesis and Characterization of Tetracyclic ETBR Antagonists

### Step 1:

To A1-CH₂—CH(NHBoc)COOMe (30 mmol, 1 eq) and iodophenylalanine (30 mmol, 1 eq) in anhydrous N,N-dimethylformamide (100 mL, 0.3M) is added N,N-diisopropylethylamine (25 mL, 150 mmol, 5 eq). The reaction mixture is then cooled to 0° C. and HATU (17 g, 45 mmol, 1.5 eq) is added. The reaction mixture is allowed to warm to room temperature (20° C.) for 18 h. The reaction mixture is poured over ice water (500 mL), filtered and dried in vacuo to afford the crude product. The crude product is then dissolved in anhydrous dichloromethane (150 mL, 0.2 M) and trifluoroacetic acid (20 mL, 300 mmol, 10 eq) is added. After stirring for 18 h at room temperature (20° C.), the reaction is quenched slowly with saturated sodium bicarbonate (200 mL). Layers are then partitioned and the aqueous is extracted a further two times with dichloromethane. The combined organic is washed with brine (100 mL) and dried over sodium sulfate. The reaction is concentrated in vacuo to yield the crude product. This residue is purified by flash chromatography (1-2% MeOH/CH₂Cl₂) to afford compound S3-1.

### Step 2:

To the compound S3-1 (2.3 mmol, 1 eq) is added ArC(═O)Cl (2.3 mmol, 1 eq), anhydrous N,N-diisopropylethylamine (5 eq) and anhydrous N,N-dimethylformamide (0.3M). The reaction mixture is then cooled to 0° C. and HATU (1.5 eq) is added. The reaction mixture is allowed to warm to room temperature (rt) for 18 h. The reaction mixture is then poured over ice water. This precipitate is filtered to afford the desired product.

The following step is performed if A1 ring contains NH group: to a stirring solution of the crude product (0.35 mmol, 1 eq) in anhydrous N,N-dimethylformamide (0.1 M), di-tert-butyl dicarbonate (1.3 eq) is added portionwise, followed by N,N-dimethylamino pyridine (0.1 eq) at 0° C. Allow to warm to rt for 18 h. The reaction mixture is quenched with saturated ammonium chloride and extracted with ethyl acetate two times. The organic layer is washed with water ten times, brine and dried over anhydrous sodium sulfate. The organic layer is then concentrated to dryness in vacuo to afford the crude product. This residue is purified by flash chromatography to afford compound S3-2.

### Step 3:

To a sealed vessel containing compound S3-2 (1 eq), borane (2 eq), [1,1′-bis(diphenylphosphino)ferrocene]dichloropalladium(II), complex with dichloromethane (0.1 eq) and potassium acetate (5 eq) is added in anhydrous N,N-dimethylformamide (0.1 M) at rt. The reaction mixture is then heated to 80° C. for 18 h. The reaction mixture is diluted with ethyl acetate and then quenched with saturated ammonium chloride. The organic layer is extracted and washed 4 times with deionized water, followed by brine. The organic layer is then dried over anhydrous sodium sulfate and concentrated to dryness in vacuo to afford the crude product. This residue is purified by flash chromatography to afford compound S3-3.

### Step 4:

The following step is performed if A1 ring contains NBoc group: to a stirring solution of compound S3-3 (1 eq) in anhydrous dichloromethane (0.2 M), trifluoroacetic acid (20 eq) is added dropwise at rt. After stirring for 18 h, the reaction mixture is quenched with saturated sodium bicarbonate and partitioned. The organic layer is washed with brine and dried over anhydrous sodium sulfate. The organic layer is then concentrated to dryness in vacuo to afford the crude product. This residue is purified by flash chromatography to afford the ester.

The ester from the previous step (or compound S3-3) is dissolved in a mixture of tetrahydrofuran, methanol and water (3:1:1, 0.067 M) and lithium hydroxide monohydrate (10 eq) is added portionwise. The reaction mixture is stirred at rt for 18 h. The reaction mixture is then concentrated to remove the organic solvents. 5% NaHSO₄ is added until a solid precipitated. The solid is collected and washed with water. Product is dried to afford compound S3-4.

### Example 4. Synthesis and Characterization of Pyrrolidine ETBR Antagonists

### Step 1:

To a stirred magnesium ethoxide (67.46 g, 590 mmol) and THF (1 100 mL) is added ethyl hydrogen malonate (1.18 moles; 145.00 mL diluted in 100 ml of THF) and the mixture is heated at 45° C. for 4 hours. Meanwhile, to a stirred benzoic acid (536 mmol) and THF (600 mL) is added 1,1′-carbonyldiimidazole (95.59 g, 589.5 mmol) in portions to avoid excess foaming. After stirring for 3 hours at room temperature this solution is added gradually to the solution of ethyl Mg-malonate. After addition the reaction mixture is heated to 45° C. After 20 hours, the reaction mixture is concentrated under reduced pressure before adding ethyl acetate (1 L) followed by 2 N HCl (500 mL). After mixing, the layers are separated and the organic phase is washed sequentially with 2 N HCl (500 mL), saturated sodium bicarbonate (500 mL), and water (500 mL). The organic phase is concentrated under reduced pressure, the residue taken up in ethyl acetate (1000 mL) and concentrated again to afford the compound S4-1.

### Step 2:

To compound S4-1 (0.104 mol), and 2-nitrovinylaryl (0.088 mol) dissolved in 180 mL of toluene and heated to 80° C. is added 1,8-diazabicyclo5.4.0]undec-7-ene (DBU, 0.65 g) with stirring. The mixture is heated until all 2-nitrovinylaryl is dissolved. The resulting solution is stirred without heating for 30 min and then an additional 0.65 g of DBU is added. After stirring an additional 1 hour, toluene (200 mL) is added, and the organic phase is washed with dilute hydrochloric acid and brine. The organic phase is dried over Na₂SO₄ and then concentrated under reduced pressure. The residue is purified using flash silica gel chromatography to afford compound S4-2 as mixture of isomers.

### Step 3:

The compound S4-2 (0.4 mol) in 500 mL of ethanol is hydrogenated under 4 atmospheres of hydrogen pressure using a Raney nickel 2800 catalyst (10 g). (The Raney nickel is washed with ethanol three times before use.) The catalyst is removed by filtration, and the Solution is concentrated under reduced pressure. The residue is purified using flash silica gel chromatography to afford desired compound. The compound from the preceding step (0.324 mol) is dissolved in 27 mL of tetrahydrofuran and 54 mL of ethanol. NaBH₃CN (2.35 g, 0.374 mol) and 5 mg bromocresol green are added. To this blue solution is added dropwise a solution of 1:2 concentrated HCl in ethanol at such a rate that the color is kept at light yellow-green. After the yellow color persisted without additional HCl, the solution is stirred for additional 20 min. The solution is concentrated in vacuo and then partitioned between chloroform and an aqueous potassium bicarbonate solution. The organic phase is separated, dried over Na₂SO₄, and concentrated under reduced pressure. The residue is purified using flash silica gel chromatography to afford compound S4-3 as mixture of isomers.

### Step 4:

Preparation of compound S4-A. To a stirred solution of aniline or amine (7.40 mmol) in methylene chloride (25 mL) at −50° C. is added successively N,N-diisopropylethylamine (1.58 mL, 8.14 mmol) and bromoacetyl bromide (0.72 mL, 7.40 mmol) such that the temperature does not exceed −40° C. On completion of the addition, the reaction mixture is allowed to warm to room temperature. After stirring for a further 30 min, the mixture is diluted with ether (70 mL) and poured into 1N sodium bisulfate solution. The phases are separated, and the upper layer is washed with water and then brine. The organic phase is dried over Na₂SO₄ and the solvent evaporated to half volume, at which point the product crystallized. The crystals are removed by vacuum filtration to afford compound S4-A.

The compound S4-3 as mixture of isomers (15.50 mmol), ethyldiisopropylamine (4.20 g, 32.56 mmol), and compound S4-A (19.0 mmol) in 30 mL of acetonitrile is heated at 50° C. for 1 hour. The solution is then concentrated in vacuo. The residue is dissolved in toluene, shaken with potassium bicarbonate solution is dried over Na₂SO₄ and concentrated in vacuo to give the product as a mixture of trans,trans- and cis,trans-ethyl esters. This mixture is dissolved in a solution of 50 mL of ethanol and 15 mL of water containing 5.00 g of sodium hydroxide and stirred for 3 hours at room temperature. The solution is concentrated in vacuo and 60 mL of water added. The mixture is extracted with ether to remove the unreacted cis,trans-ethyl ester. The aqueous phase is treated with hydrochloric acid until slightly cloudy. It is then further neutralized with acetic acid to give the crude acid product. The crude product is filtered and purified by dissolving it in tetrahydrofuran, drying over Na₂SO₄, concentrating in vacuo, and crystallizing from ether to give compound S4-4.

### Example 5. Synthesis and Characterization of Pyrimidine ETBR Antagonists

### Step 1:

Phenol (40.40 mol) is added dropwise over 15 min to a sodium methoxide solution (1.0M methanol solution, 395 mL) at 0° C. After being stirred for 15 min, dimethyl chloromalonate (75.0 g, 0.45 mol) is added dropwise over 15 min at the same temperature. The reaction mixture is stirred for 20 h at rt and concentrated. Water is added to the mixture, and the aqueous layer is extracted with toluene. The organic layer is washed with 1% aqueous NaOH solution and saturated NaCl, dried, and concentrated. The residue is distilled under reduced pressure to give crude dimethylphenoxymalonate, which is used in a following reaction without further purification. Formamidine acetate (8.6 g, 82.6 mmol) and the malonate (55.1 mmol) from the preceding reaction were added to a sodium methoxide solution (1.0 M methanol solution, 165 mL) at 0° C. The reaction mixture is stirred for 2.5 h at rt and concentrated. Water is added to the mixture, and the aqueous layer is extracted with toluene. The aqueous layer is acidified with 1 N HCl. The resulting precipitate is collected, washed with water and dried to give compound S5-1.

### Step 2:

To a mixture of S5-1 (46.1 mmol) and collidine (15.0 mL, 113.5 mmol) is added phosphorous oxychloride (62.6 mL, 6.7 mol) portionwise at 0 C. The reaction mixture is stirred at 135° C. for 4 h. The cooled reaction mixture is poured into ice-water and extracted with AcOEt. The organic layer is washed with aqueous NaHCO₃ solution and saturated NaCl solution, dried and concentrated. The residue is purified by silica gel chromatography eluting with hexane to give S5-2.

### Step 3:

A solution of S5-2 (19.5 mmol) and potassium arylsulfonate (40.0 mmol) in DMSO (25 mL) is stirred at 120° C. for 30 min. 1N HCl is added, and the mixture is extracted with AcOEt. The organic layer is washed with water, dried and concentrated. The residue is crystallized with MeOH to give compound S5-3.

### Step 4:

Sodium hydride (60% dispersion in mineral oil, 2.0 g, 50.0 mmol) is added to 1,4-butandiol (40 mL), and the mixture is stirred at rt for 30 min. Compound S5-3 (16.7 mmol) is added, and the reaction mixture is stirred at 100° C. for 6.5 h. 1N HCl is added, and the mixture is extracted with AcOEt. The organic layer is washed with water and saturated NaCl solution, dried and concentrated. The residue is purified by silica gel chromatography eluting with hexane/AcOEt to give compound S5-4.

### Example 6. Synthesis and Characterization of Heteroaryl ETBR Antagonists

### Step 1:

Sodium carbonate (5 ml, 2M aqueous solution) followed by arylboronic acid (2.4 mmol) are added to a solution of heteroarylbromide (2 mmol) and Pd(PPh₃)₄ (100 mg) in toluene (5 ml) and ethanol (5 ml) under nitrogen. The mixture is refluxed for 2 hours, cooled to room temperature, and extracted with ethyl acetate (2×50 ml). The combined organic layers is dried over MgSO₄ and evaporated. The residue is purified using flash silica gel chromatography to afford compound S6-1.

### Step 2:

To an ice-cold solution of S6-1 (5 mmol) is added chlorosulfonic acid (0.33 ml, 5 mmol) over a 15 min with constant stirring. After 10 min, phosphorous oxychloride (2 ml) and phosphorous pentachloride (1 ml) are added. The reaction mixture is slowly allowed to attain ambient temperature and stirred for 3 hours. The mixture is then poured onto crushed ice (50 g) and is extracted with ethyl acetate (2×50 ml). The combined organic layers is dried over MgSO₄ and evaporated. The residue is purified using flash silica gel chromatography to afford compound S6-2.

### Step 3:

A solution of amine (1.0 mmol) in dry THF (2 ml) is added to a suspension of sodium hydride (60% dispersion in mineral oil, 90 mg, 2.2 mmol) in dry THF (1 ml) at 0°−5° C. After stirring at 0°−5° C. for 5 min, the reaction is warmed to room temperature for 10 min to complete the reaction. The reaction mixture is re-cooled to 0° C. and compound S6-2 (1.1 mmol), which had been dissolved in dry THF (2 ml), is added slowly. Stirring is continued for 1 h and during this period the reaction mixture slowly attained ambient temperature. THF is removed under reduced pressure. The residue is dissolved in water (10 ml), the pH is adjusted to 2-3 by adding concentrated HCl, and is extracted with methylene chloride (3×10 ml). The combined organic layers are dried over anhydrous magnesium sulfate and concentrated under reduced pressure to give crude product. The pure compound S6-3 is obtained by recrystallization using hexanes/ethyl acetate.

### Example 7. Biological Activities of ETBR Antagonists

Combination of an ETBR Antagonist and an Immune Checkpoint Inhibitor Eradicated Tumor.

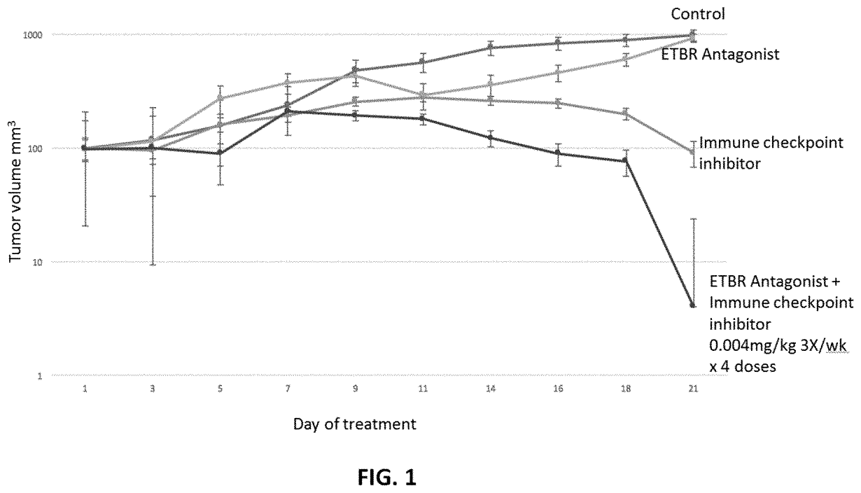

FIG. 1 is an in vivo tumor growth curve over the time course of 21 days, which shows that a dual combination of another ETBR antagonist and an immune checkpoint inhibitor resulted in unexpected superior efficacy relative to the ETBR antagonist alone or the immune checkpoint inhibitor alone in a SM1 model. This model did not respond well to anti-PD1 or this ETBR antagonist as a single agent. However, when the immune checkpoint inhibitor was combined with the ETBR antagonist, as shown in the purple curve, there was tumor shrinkage below baseline by the end of the study. Immunohistochemical analysis revealed that in fact the tumors had been completely eradicated leaving tumor remnants composed of mature adipose tissue.

Intratumoral (Internal) TLO Formation with Broad Dosing Range of ETBR Antagonists.

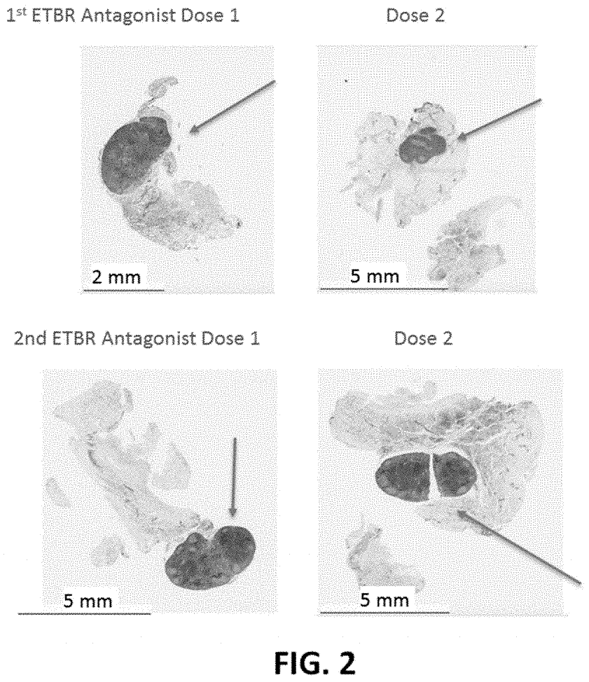

FIG. 2 shows that strikingly tumor remnants after treatment of two ETBR antagonists respectively in combination with an immune checkpoint inhibitor had intratumoral TLOs (tertiary lymphoid organs). TLOs can be functionally equivalent to lymph nodes that produce anti-tumor T and B cells for long lasting anti-tumor immunity.

ETBR Antagonists in Combination with an Immune Checkpoint Inhibitor Demonstrate Synergistic Results.

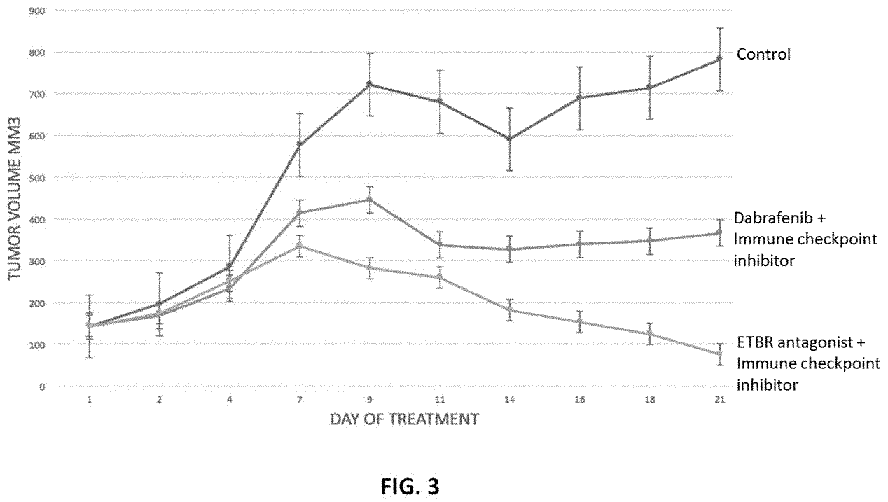

Dual combination of ETBR antagonists and immune checkpoint inhibitors (FIG. 3) resulted in superior efficacy relative combinations with approved cancer drugs. The syngenic melanoma model V600E+ (BRAF mutated) SM1 tumor model were used in C57BL/6 mice to assess efficacy of ETBR antagonists disclosed herein in combination with immune checkpoint inhibitors as compared to a standard treatment, dabrafenib with an immune checkpoint inhibitor. Previous studies have indicated that V600E+ model demonstrates no efficacy for an immune checkpoint inhibitor as a single agent (and little tumor infiltrating lymphocytes (TILs)). In this study 6-8 week old female C57BL/6 mice were inoculated with SM1 tumor fragments (TME* components present). The general dosing schemes were as follows: dabrafenib (e.g., 30 mg/kg daily by oral gavage), immune checkpoint inhibitors (e.g., anti-PD1, anti-PD-L1, anti-CTLA-4, 10 mg/kg Q4D IP beginning 2 days after dabrafenib), ETBR antagonist (4 μg administered QOD IV beginning 2 days after dabrafenib). Tumors were measured three times per week, and the study was terminated, e.g., after 21 days of dosing and IHC analysis of tumors was performed. The dual combination of the immune checkpoint inhibitors and the ETBR antagonist induced tumor shrinkage below baseline. In stark contrast, a standard combination of dabrafenib and the immune checkpoint inhibitors failed to shrink tumors but demonstrated intermediate tumor growth inhibition. IHC analysis of tumors treated with immunotherapeutics and ETBR antagonists revealed that tumors had been eradicated leaving only residual adipose tissue. In sum, the combination of the immune checkpoint inhibitors with the ETBR antagonists as described herein provided significant improvement against tumor growth relative to the existing therapeutic paradigm.

Dual Combination ETBR Antagonists and Immune Checkpoint Inhibitors Eradicates Tumors.

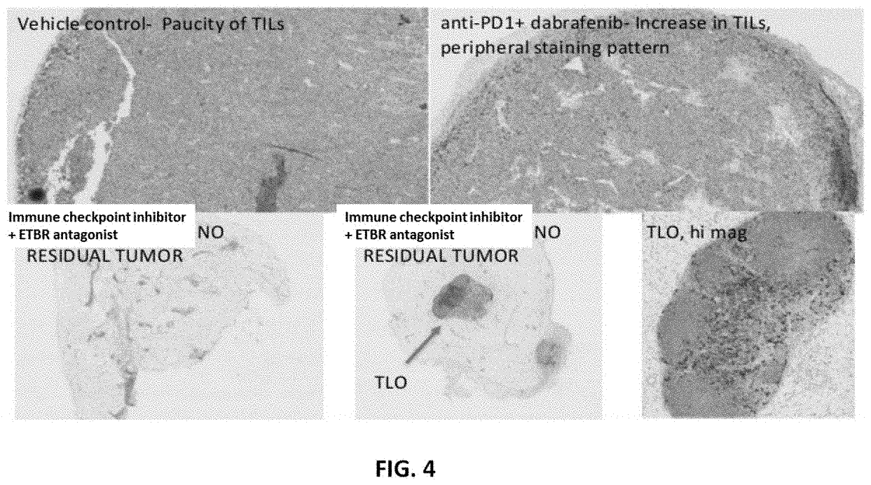

FIG. 4 demonstrates the results of histological examination of V600E+ melanoma tumor cells implanted into C57BL/6 mice 21 days after treatment as indicated in FIG. 3. Dual combinations of ETBR antagonists herein and immune checkpoint inhibitors resulted in superior efficacy relative combinations with approved cancer drugs. The ETBR antagonists and immune checkpoint inhibitors (e.g., anti-PD1, anti-PD-L1, anti-CTLA-4) combination therapy eradicate the tumors, e.g., in 21 days, promote robust infiltration by CD8+ lymphocytes (TILs), and tertiary lymphoid organ (TLO) formation. TIL infiltration is exemplified by the dark punctate staining. TLOs are functionally equivalent to lymph nodes, produce tumor-specific T- and B-cells, and induce long lasting anti-tumor immunity. TLOs are functionally equivalent to lymph nodes, produce tumor specific T- and B-cells, induce long lasting anti-tumor immunity, and are associated with favorable clinical prognosis in multiple cancers.

Intratumoral TLO Formation Induced by Combination Therapy Including ETBR Antagonists and Immune Checkpoint Inhibitors.

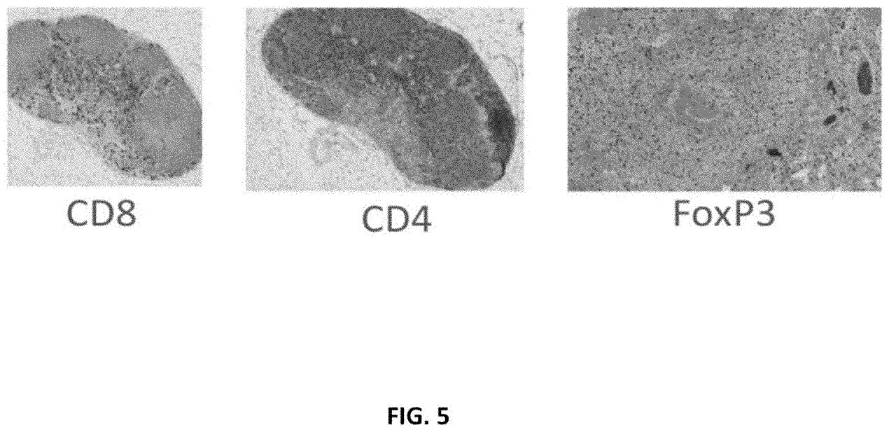

FIG. 5 demonstrates the histological examination of V600E+ melanoma tumor cells implanted into C57BL/6 mice 21 days after treatment as indicated in FIG. 3 with ETBR antagonists and immune checkpoint inhibitors combination therapy. The staining of CD8+, CD4+ and Treg (FoxP3) lymphocytes (dark punctate staining) indicates that the combination therapy promotes strong mobilization of lymphocytes to the tumor, which is associated with tumor eradication and positive patient outcomes.

Intratumoral (Internal) TLO Formation Associated with Treatment with ETBR Antagonists.

The results were obtained with combination therapies (two- and three-part), TLO formation and efficacy for tumor eradication. The data indicated that (i) internal TLO formation is associated with tumor eradication; and (ii) the combination of immune checkpoint inhibitors and ETBR antagonists is associated with intratumoral TLO formation and tumor reduction. The inclusion of ETBR antagonists with immune checkpoint inhibitors is synergistic and appears to help restore sensitivity to immune checkpoint inhibitors. The addition of dabrafenib to anti-PD1/ETBR antagonist combination impairs efficacy, possibly due to dabrafenib's ability to increase Tregs and tumor-associated macrophages (TAMs).

ETBR Antagonists at 0.6 μg in Combination with Immune Checkpoint Inhibitors and Dabrafenib Promotes Diffuse CD8+ TIL Staining.

Disclosed herein is a histological examination of V600E+ melanoma tumor cells implanted into C57BL/6 mice 21 days after treatment with the respective combination therapy. The diffuse distribution of CD8+ TIL staining (dark punctate staining) appears to be associated with higher efficacy as compared to those with peripheral distribution of TILs.

Thus, ETBR antagonists described herein demonstrate synergistic activity with immunotherapeutics such as immune checkpoint inhibitors in a preclinical melanoma model in which the immune checkpoint inhibitors lacks any efficacy as a single agent. Tumor reduction or eradication correlates well with intratumoral TLO formation or neogenesis, and diffuse infiltration pattern of TILs rather than tumor-peripheral TIL distribution. TLO neogenesis has prognostic implications and correlates will with increased patient survival. The dual combination of ETBR antagonists and anti-oncologic agents is superior to other dual and triple combinations in terms of (i) anti-tumor efficacy; (ii) low anticipated toxicity (based upon established safety profile of parent compound in humans); and (iii) overall treatment cost (relative to triple therapies).

Determination of ETBR inhibitory effect for ETBR antagonists. ETBR antagonists disclosed herein inhibit tumor (e.g., melanoma) growth and metastasis, and induce apoptosis in tumor cells (e.g., melanoma tumor cells). Cellular agonist effect is calculated as a % of control response to a known reference agonist for Endothelin B, and cellular antagonist effect is calculated as a % inhibition of control reference agonist response for Endothelin B. Results showing ≥25% inhibition of agonist effect are considered significant. In in vitro studies, the ETBR antagonists herein show smaller IC50 values or Kd relative to BQ-788. In in vivo pharmacological studies, the ETBR antagonists herein show enhanced biologic activity relative to BQ-788.

Plasma Concentrations of ETBR Antagonists Versus BQ-788.

ETBR antagonists disclosed herein show enhanced plasma concentrations relative to BQ-788. Briefly, rats are administered either BQ-788 or an ETBR antagonist herein via intravenous infusion. Plasma samples are collected at various time points and ET-1 ELISA performed. BQ788 is a peptide drug that can be rapidly degraded in plasma and thus drug levels are difficult to detect directly. The binding of BQ788 to ETBR can result in an increase in plasma concentrations of ET-1, the ligand for ETBR. As such, plasma levels of ET-1 are commonly used as an indirect measure of BQ-788 biologic activity. The ETBR antagonists show an enhanced duration and amplitude of response relative to BQ-788 as exemplified by the prolonged peak out to about an half hour or several hours as compared to BQ-788.

ETBR Antagonists Inhibit Tumor Growth and Metastasis.

ETBR antagonists disclosed herein induce apoptosis in tumor cells (e.g., melanoma tumor cells). Mice are implanted with tumor cells, e.g., 1×10⁶ SKMEL28 human melanoma cells. Tumors are established for 10 days until palpable, and then the ETBR antagonists (e.g., dissolved in DMSO) is administered to the mice, e.g., injected 3 times per week for 6 weeks. Mice are then sacrificed, lungs are harvested, and tumors are weighed. A significant reduction in tumor weight is observed. Lung specimens harvested from control mice show numerous metastases whereas specimens harvested from mice treated with the ETBR antagonists show a high clearance (e.g., <95%) of lung metastases. A low side effect profile of ETBR antagonists, e.g., by injection administration such as subcutaneous or intravenous, makes feasible the treatment of both advance and earlier stage tumor patients.

### Example 8. Treatment of Melanoma in a Human Subject

A human patient suffering melanoma, e.g., malignant melanoma or metastatic melanoma, is administered compounds or pharmaceutical compositions according to a method for treatment disclosed herein. The treatment cures the patient or ameliorates the patient's one or more symptoms such as a sore, spread of pigment from the border of a spot into surrounding skin, redness or a new swelling beyond the border of the mole, change in sensation, such as itchiness, tenderness, or pain, or change in the surface of a mole—scaliness, oozing, bleeding, or the appearance of a lump or bump.

### Example 9. Treatment of a Malignant Solid Tumor in a Human Subject

A human patient suffering a malignant solid tumor, e.g., pancreatic tumor, ovarian tumor, sarcomas, carcinomas, and lymphomas, is administered compounds or pharmaceutical compositions according to a method for treatment disclosed herein. The treatment reduces a tumor volume or mass, or eradicates the tumor in the patient.

### Example 10. Treatment of Squamous Cell Carcinoma in a Human Subject

A human patient suffering squamous cell carcinoma is administered compounds or pharmaceutical compositions according to a method for treatment disclosed herein. The treatment cures the patient or ameliorates the patient's one or more symptoms such as firm red nodule, flat sore with a scaly crust, new sore or raised area on an old scar or ulcer, rough scaly path on a lip or inside a mouth, scaly red patches, open sores, or warts or elevated growths with a central depression on or in anus on genitals.

### Example 11. Treatment of Glioblastoma in a Human Subject

A human patient suffering glioblastoma is administered compounds or pharmaceutical compositions according to a method for treatment disclosed herein. The treatment cures the patient, reduces or eradicates brain tumor, or ameliorates the patient's one or more symptoms such as headache, nausea, vomiting, memory loss, drowsiness, blurred vision, change to personality, mood, or concentration, localized neurological problems, or seizure.

### Example 12. Treatment of a Pancreatic Cancer in a Human Subject

A human patient suffering a pancreatic cancer is administered compounds or pharmaceutical compositions according to a method for treatment disclosed herein. The treatment cures the patient or ameliorates the patient's one or more symptoms such as Jaundice, light-colored stools, dark urine, pain in the upper or middle abdomen and back, weight loss, appetite loss, or fatigue.

### Example 13. Treatment of an Ovarian Cancer in a Human Subject

A human patient suffering an ovarian cancer is administered compounds or pharmaceutical compositions according to a method for treatment disclosed herein. The treatment cures the patient or ameliorates the patient's one or more symptoms for example: abdominal bloating, indigestion or nausea, changes in appetite such as a loss of appetite or feeling full sooner, pressure in the pelvis or lower back, a frequent or urgent need to urinate and/or constipation, changes in bowel movements, increased abdominal girth, tiredness or low energy, or changes in menstruation.

### Example 14. A Single-Center, Open-Label, Phase 1 Study in Subjects with Newly Diagnosed Glioblastoma

This is a prospective, single-center, open-label, 3+3 dose escalation Phase 1 safety study. Adults with newly diagnosed glioblastoma or gliosarcoma receive compounds or pharmaceutical compositions herein in addition to or in lieu of the standard of care treatment for glioblastoma. The study consists of a screening period, a treatment period, and a 30-day safety follow up period. The study will end when the last treated subject has completed study treatment and the 30-day safety follow-up period. The planned duration of the study is approximately 34-38 months depending on the number of dose levels and cohorts of subjects enrolled. Subject participation in the study is for approximately 16 months.

Primary Outcome Measures:
1. Number of subjects with dose-limiting toxicities observed during the first 10 weeks of study treatment. [Time Frame: Start of treatment to week 10]

Secondary Outcome Measures:
1. Plasma concentrations of endothelin-1 [Time Frame: Baseline, Weeks 2, 6, and 10]
2. Plasma concentrations of therapeutic compounds and metabolites [Time Frame: Baseline, Weeks 2 and 6]
3. Area under the plasma concentration-time curve (AUCτ) during one dosing interval for treated subjects [Time Frame: Week 4]
4. Peak plasma concentration (C max) during one dosing interval for treated subjects [Time Frame: Week 4]
5. Time to reach peak plasma concentration (T max) during one dosing interval for treated subjects [Time Frame: Week 4]
6. Number of adverse events (per Common Terminology Criteria for Adverse Events [CTCAE] criteria, version 4.03]) leading to premature discontinuation of study treatment [Time Frame: Starting from first dose until the end of treatment plus 30 days of follow-up]
7. Number of subjects with marked laboratory abnormalities or abnormal electrocardiogram (ECG) findings [Time Frame: Starting from first dose until the end of treatment plus 30 days of follow-up]
8. Change from baseline in pulse rate, systolic & diastolic blood pressure [Time Frame: Starting from first dose until the end of treatment plus 30 days follow-up]
9. Exploratory efficacy endpoint of proportion of subjects with progression free survival (PFS) at 6 and 12 months [Time Frame: 6 and 12 months after the start of treatment]
10. Number of adverse events (per CTCAE] criteria, version 4.03]) as a measure of safety and tolerability. [Time Frame: Starting from first dose until the end of treatment plus 30 days of follow-up]

Eligibility Criteria

Inclusion Criteria:

Subjects at least 18 years of age
Histologically proven supratentorial GBM or gliosarcoma
Use of effective contraception by women of childbearing potential.
Use of effective contraception by fertile males with a female partner of childbearing potential.
Interval of at least 3 weeks after biopsy or open surgery and able to begin study treatment.
Result from a post-operative contrast-enhanced brain MRI within 72 hours after surgery or biopsy.
Adequate bone marrow function
Karnofsky Performance Score of at least 70.

Exclusion Criteria:

Prior treatment for glioblastoma or gliosarcoma.
Evidence of leptomeningeal spread of glioblastoma or gliosarcoma.
Tumor foci below the tentorium or beyond the cranial vault.
Evidence of recent hemorrhage on post-operative contrast enhanced brain MRI (except hemosiderin, resolving hemorrhage changes related to surgery, presence of punctuate hemorrhage in tumor).
Aspartate aminotransferase or alanine aminotransferase >3 times the upper limit of normal. Supine systolic blood pressure <100 mmHg or diastolic blood pressure <50 mmHg.
Medical history of orthostatic hypotension.
International normalized ratio >1.5 on anticoagulant therapy, active bleeding on low molecular weight heparin, or chronic condition with a high risk of bleeding.
Severe renal impairment.
Severe hepatic impairment.
Severe, active co-morbidity: (e.g. cardiac disease; respiratory disease; chronic hepatitis; hemtological and bone marrow diseases; severe malabsoprtion; human immunodeficiency virus).
No concurrent strong CYP3A4 inducers or inhibitors.
No investigational drug within 4 weeks of starting study treatment.
Any life-threatening condition that could affect protocol compliance.

### Example 15. Clinical Trial of Intralesional Administration to Melanoma Skin Metastases

This human study is aimed to check whether safety and preclinical results obtained by intratumoral administration of compounds or pharmaceutical compositions herein can be repeated in human melanoma patients.

Methods. Three patients receive a single intralesional application, e.g., 3 mg. After 3-7 days, the lesions are measured and removed for analysis. The administered dose is increased to a cumulative dosage, e.g., 8 mg in patient 4 (4×2.0 mg, days 0-3; lesion removed on day 4) and e.g., to 10 mg in patient 5 (3×3.3 mg, days 0, 3, and 10; lesion removed after 14 days). Control lesions are simultaneously treated with phosphate-buffered saline (PBS). All samples are processed and analyzed without knowledge of the clinical findings.

Results. No adverse event is observed, regardless of administered dose. All observations are in accordance with results obtained in preclinical studies. Accordingly, no difference in degree of tumor necrosis is detected between ETBR antagonist- and PBS-treated samples. Indication of efficacy is observed, consisting of both direct effects (decreased expression of endothelin receptor B and of survival factors, reduced proliferation) and indirect effects (enhanced immune cell infiltration and angiogenesis). Importantly, semiquantitatively scored immunohistochemistry for CD31 and CD3 reveals more blood vessels and lymphocytes, respectively, in treated tumors. Also, in all patients, inverse correlation is observed in expression levels between ETBR and HIF1A. Finally, in the patient treated for longer than 1 week, inhibition in lesion growth is observed as shown by size measurement.

Conclusion. The intralesional applications are well tolerated and show signs of directly and indirectly reducing the viability of melanoma cells.

While some embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of forming a tertiary lymphoid organ (TLO) in a subject in need thereof, comprising administering to the subject an ETBR antagonist.

2. The method of claim 1, wherein the ETBR antagonist is BQ-788, A192621, A-308165, IRL-1038, IRL-2500, RO-468443, BQ-017, or a structural analog thereof.

3. The method of claim 2, wherein the ETBR antagonist is BQ-788 or a structural analog thereof.

4. The method of any one of claims 1-3, wherein the ETBR antagonist is formulated as a controlled, or delayed release formulation.

5. The method of claim 4, wherein the ETBR antagonist is formulated as nanoparticles.

6. The method of any one of claims 1-5, wherein the ETBR antagonist is a non-deuterated BQ-788 analog.

7. The method of any one of claims 1-6, further comprising administering to the subject an additional therapeutic agent.

8. The method of claim 7, wherein the additional therapeutic agent is an anti-oncologic therapeutic agent or an anti-bacterial or an antimicrobial therapeutic agent.

9. The method of claim 8, wherein the additional therapeutic agent is an agent used to reduce transplant rejection.

10. The method of claim 9, wherein the additional therapeutic agent is an immune suppressant or an anti-CD40 agent.

11. The method of any one of claims 7-10, wherein the additional therapeutic agent is an anti-oncologic therapeutic agent.

12. The method of claim 11, wherein the anti-oncologic agent comprises a bRAF inhibitor, an immune checkpoint inhibitor, a caspase-8 inhibitor, an ETAR antagonist, niacinamide, a chemotherapeutic agent, or any combination thereof.

13. The method of claim 12, wherein the anti-oncologic agent comprises an immune checkpoint inhibitor.

14. The method of claim 13, wherein the immune checkpoint inhibitor comprises at least one anti-PD1 antibody, at least one anti-PD-L1 antibody, at least one anti-CTLA4 antibody, or any combination thereof.

15. The method of claim 14, wherein the at least one anti-PD1 antibody comprises pidilizumab, BMS-936559, nivolumab, pembrolizumab, or any combination thereof.

16. The method of claim 14 or 15, wherein the at least one anti-PD-L1 antibody comprises atezolizumab, avelumab, durvalumab, MDX-1105, or any combination thereof.

17. The method of any one of claims 7-16, wherein the ETBR antagonist and the additional therapeutic agent are administered at different times.

18. The method of claim 17, wherein the ETBR antagonist is administered 2, 3, 4, or 5 times the frequency of the additional therapeutic agent.

19. The method of claim 18, wherein the ETBR antagonist is administered 3 times frequently as the additional therapeutic agent.

20. The method of claim 19, wherein the ETBR antagonist is administered 3 times every 2-3 weeks and the additional therapeutic agent is administered 1 time about every 2-3 weeks.

21. The method of claim 20, wherein the ETBR antagonist is administered 3 times about every 21 days and the additional therapeutic agent is administered 1 time about every 21 days.

22. The method of any one of claims 1-21, wherein the tertiary lymphoid organ is formed within or adjacent to a tumor.

23. The method of claim 22, wherein the tumor is a solid tumor, melanoma tumor, solid tumor cancer, malignant melanoma, metastatic melanoma, malignant squamous cell carcinoma, metastatic squamous cell carcinoma, glioblastoma, brain cancer, pancreatic cancer, colon cancer, breast cancer, ovarian cancer, prostate cancer, or any combination thereof.

24. The method of any one of claims 1-23, wherein the subject is a human.

25. The method of any one of claims 1-24, wherein the subject is resistant to an immunotherapy before the treatment.

26. The method of any one of claims 1-25, wherein the administration restores Tumor Infiltrating Lymphocytes (TILs) in a tumor microenvironment.

27. A compound of Formula (4): 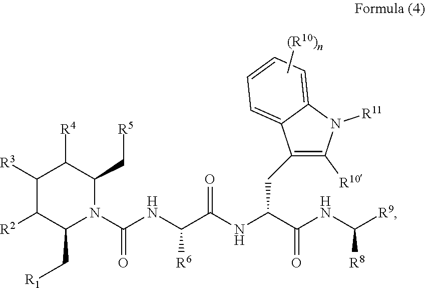
a stereoisomer thereof, or a pharmaceutically acceptable salt or solvate thereof,
wherein,
each of R¹-R⁵ is independently hydrogen, deuterium, halogen, hydroxy, amino, nitro, cyano, C₁-C₈ alkyl, C₂-C₈ alkenyl, C₂-C₈ alkynyl, C₃-C₈ cycloalkyl, C₁-C₈ alkoxy, C₁-C₈ haloalkyl, aryl, or heteroaryl;
R⁶ is C₁-C₈ alkyl, C₂-C₈ alkenyl, C₂-C₈ alkynyl, C₃-C₈ cycloalkyl, C₁-C₈ alkoxy, C₁-C₈ haloalkyl, aryl, or heteroaryl, wherein R⁶ optionally comprises deuterium;
R⁸ and R⁹ are each independently C₁-C₈ alkyl, C₂-C₈ alkenyl, C₂-C₈ alkynyl, C₃-C₈ cycloalkyl, C₁-C₈ alkoxy, C₁-C₈ haloalkyl, aryl, heteroaryl, or —COOR′, or R⁸ and R⁹ may be taken together to form substituted or unsubstituted C₃-C₈ cycloalkyl, substituted or unsubstituted C₂-C₇ heterocycloalkyl, substituted or unsubstituted aryl, substituted or unsubstituted heteroaryl, or substituted or unsubstituted polycyclic ring system, wherein R⁸ or R⁹ each optionally comprises deuterium;
R¹⁰ and R^{10′} are each independently hydrogen, deuterium, halogen, hydroxy, amino, nitro, cyano, C₁-C₈ alkyl, C₂-C₈ alkenyl, C₂-C₈ alkynyl, C₃-C₈ cycloalkyl, C₁-C₈ alkoxy, C₁-C₈ haloalkyl, aryl, or heteroaryl;
R¹¹ is hydrogen, deuterium, C₁-C₈ alkyl, C₁-C₈ haloalkyl, C₃-C₈ cycloalkyl, aryl, heteroaryl, or —COOR′;
each R′ is independently hydrogen or C₁-C₈ alkyl; and
n is an integer from 0-4.

28. The compound of claim 27, wherein R², R³, and R⁴ are hydrogen, and wherein R¹ and R⁵ are methyl.

29. The compound of claim 27 or 28, wherein R⁶ is —(CH₂)C(CH₃)₃.

30. The compound of any one of claims 27-29, wherein R¹⁰ and R^{10′} are hydrogen, and wherein R¹¹ is —COOCH₃.

31. The compound of any one of claims 27-30, wherein R⁸ is —(CH₂)₃CH₃.

32. The compound of claim 27, wherein the compound is selected from the group consisting of: 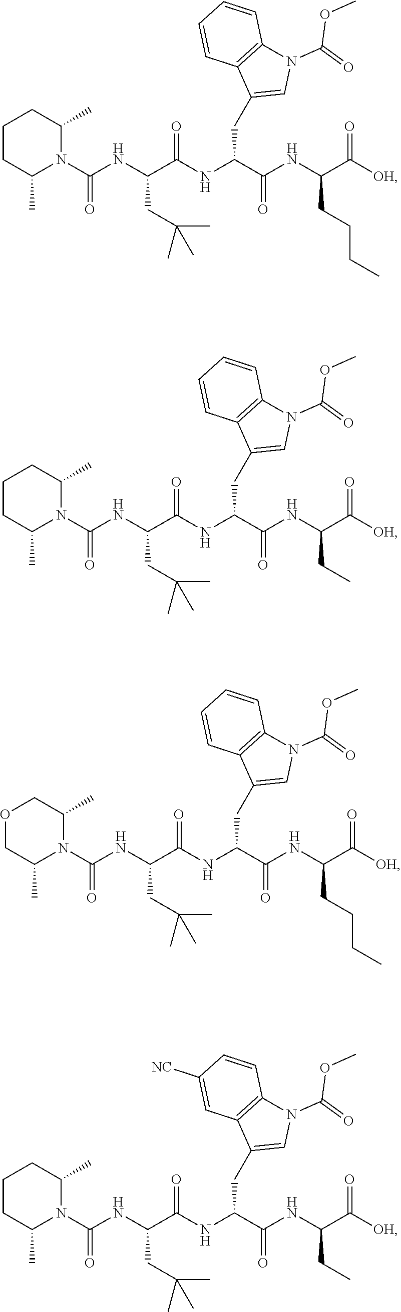 [SMILES: CCCC[C@@H](NC(=O)[C@@H](Cc1cn(C(=O)OC)c2ccccc12)NC(=O)[C@H](CC(C)(C)C)NC(=O)N1[C@H](C)CCC[C@@H]1C)C(=O)O ; CC[C@@H](NC(=O)[C@@H](Cc1cn(C(=O)OC)c2ccccc12)NC(=O)[C@H](CC(C)(C)C)NC(=O)N1[C@H](C)CCC[C@@H]1C)C(=O)O ; CCCC[C@@H](NC(=O)[C@@H](Cc1cn(C(=O)OC)c2ccccc12)NC(=O)[C@H](CC(C)(C)C)NC(=O)N1[C@H](C)COC[C@@H]1C)C(=O)O ; CC[C@@H](NC(=O)[C@@H](Cc1cn(C(=O)OC)c2ccc(C#N)cc12)NC(=O)[C@H](CC(C)(C)C)NC(=O)N1[C@H](C)CCC[C@@H]1C)C(=O)O] 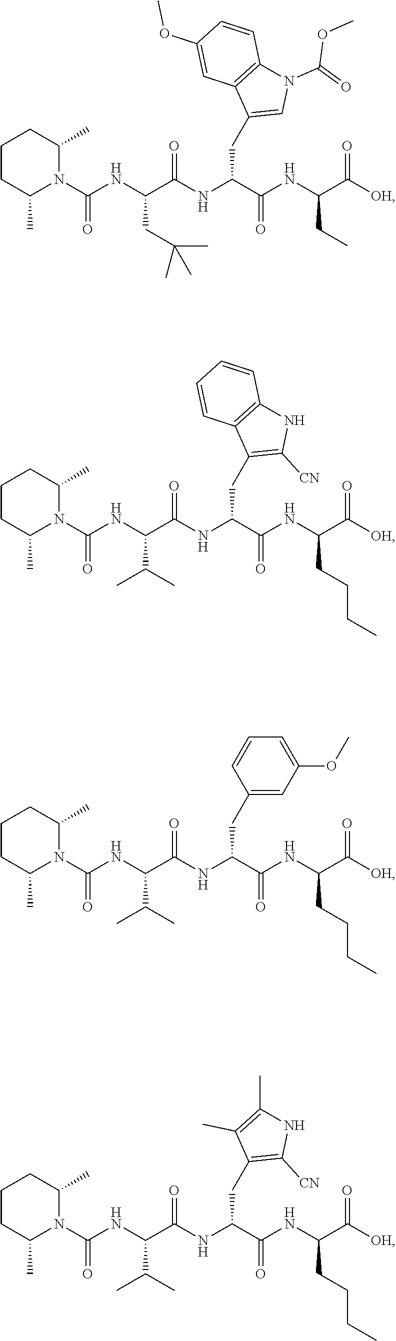 [SMILES: CC[C@@H](NC(=O)[C@@H](Cc1cn(C(=O)OC)c2ccc(OC)cc12)NC(=O)[C@H](CC(C)(C)C)NC(=O)N1[C@H](C)CCC[C@@H]1C)C(=O)O ; CCCC[C@@H](NC(=O)[C@@H](Cc1c(C#N)[nH]c2ccccc12)NC(=O)[C@@H](NC(=O)N1[C@H](C)CCC[C@@H]1C)C(C)C)C(=O)O ; CCCC[C@@H](NC(=O)[C@@H](Cc1cccc(OC)c1)NC(=O)[C@@H](NC(=O)N1[C@H](C)CCC[C@@H]1C)C(C)C)C(=O)O ; CCCC[C@@H](NC(=O)[C@@H](Cc1c(C#N)[nH]c(C)c1C)NC(=O)[C@@H](NC(=O)N1[C@H](C)CCC[C@@H]1C)C(C)C)C(=O)O] 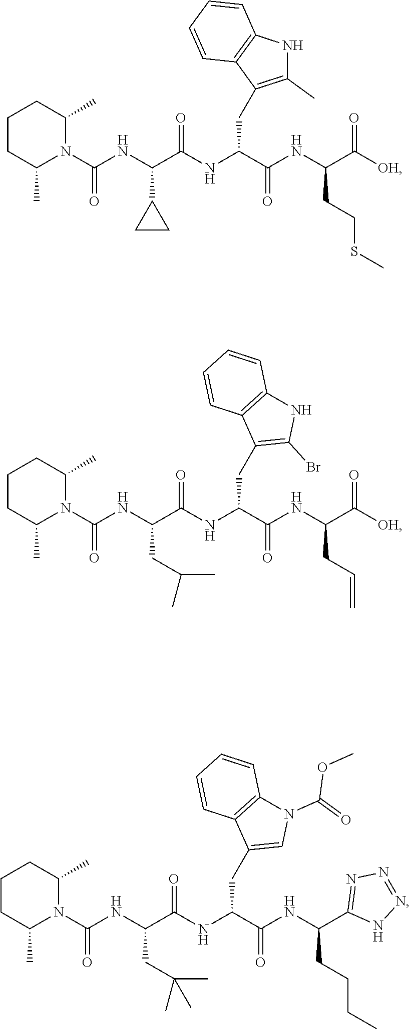 [SMILES: CSCC[C@@H](NC(=O)[C@@H](Cc1c(C)[nH]c2ccccc12)NC(=O)[C@@H](NC(=O)N1[C@H](C)CCC[C@@H]1C)C1CC1)C(=O)O ; C=CC[C@@H](NC(=O)[C@@H](Cc1c(Br)[nH]c2ccccc12)NC(=O)[C@H](CC(C)C)NC(=O)N1[C@H](C)CCC[C@@H]1C)C(=O)O ; CCCC[C@@H](NC(=O)[C@@H](Cc1cn(C(=O)OC)c2ccccc12)NC(=O)[C@H](CC(C)(C)C)NC(=O)N1[C@H](C)CCC[C@@H]1C)c1nnn[nH]1]
a stereoisomer thereof, a deuterated analog thereof, a fluorinated analog thereof, and a pharmaceutically acceptable salt or solvate thereof.

33. A compound of Formula (5): 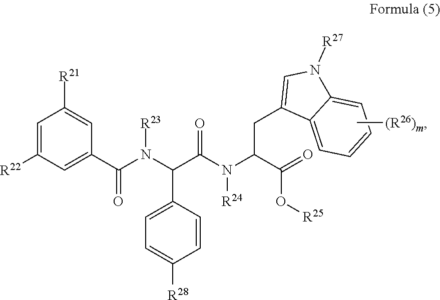
or a pharmaceutically acceptable salt or solvate thereof,
wherein,
each R²¹ and R²² is independently hydrogen, deuterium, halogen, hydroxy, amino, nitro, cyano, C₁-C₆ alkyl, C₃-C₈ cycloalkyl, or C₁-C₆ alkoxy;
each R²³ and R²⁴ is independently hydrogen or C₁-C₄ alkyl;
R²⁵ is hydrogen or C₁-C₆ alkyl;
each R²⁶ is independently deuterium, halogen, hydroxy, amino, nitro, cyano, C₁-C₆ alkyl, C₂-C₆ alkenyl, C₂-C₆ alkynyl, C₃-C₈ cycloalkyl, C₁-C₆ alkoxy, C₁-C₆ haloalkyl, aryl, or heteroaryl;
R²⁷ is hydrogen, deuterium, C₁-C₆ alkyl, C₁-C₆ haloalkyl, C₃-C₈ cycloalkyl, aryl, heteroaryl, or —COOR²⁹;
R²⁸ is substituted or unsubstituted C₃-C₈ cycloalkyl, substituted or unsubstituted C₂-C₇ heterocycloalkyl, substituted or unsubstituted aryl, or substituted or unsubstituted heteroaryl;
R²⁹ is hydrogen or C₁-C₆ alkyl; and
m is an integer from 0-4.

34. The compound of claim 33, wherein the compound is selected from the group consisting of: 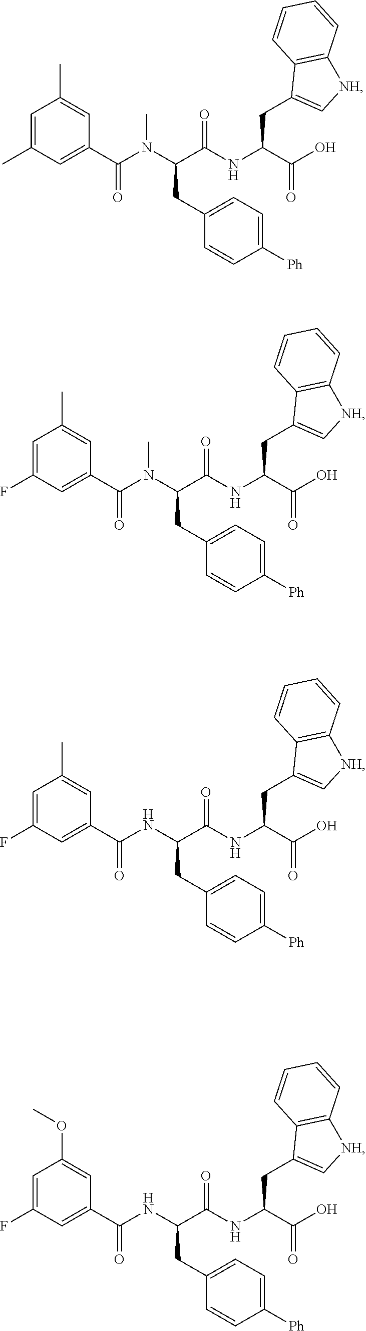 [SMILES: Cc1cc(C)cc(C(=O)N(C)[C@H](Cc2ccc(-c3ccccc3)cc2)C(=O)N[C@@H](Cc2c[nH]c3ccccc23)C(=O)O)c1 ; Cc1cc(F)cc(C(=O)N(C)[C@H](Cc2ccc(-c3ccccc3)cc2)C(=O)N[C@@H](Cc2c[nH]c3ccccc23)C(=O)O)c1 ; Cc1cc(F)cc(C(=O)N[C@H](Cc2ccc(-c3ccccc3)cc2)C(=O)N[C@@H](Cc2c[nH]c3ccccc23)C(=O)O)c1 ; COc1cc(F)cc(C(=O)N[C@H](Cc2ccc(-c3ccccc3)cc2)C(=O)N[C@@H](Cc2c[nH]c3ccccc23)C(=O)O)c1] 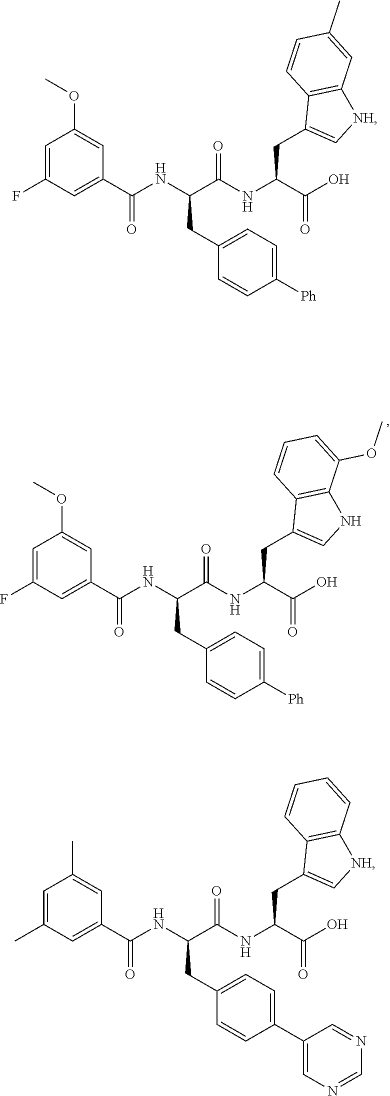 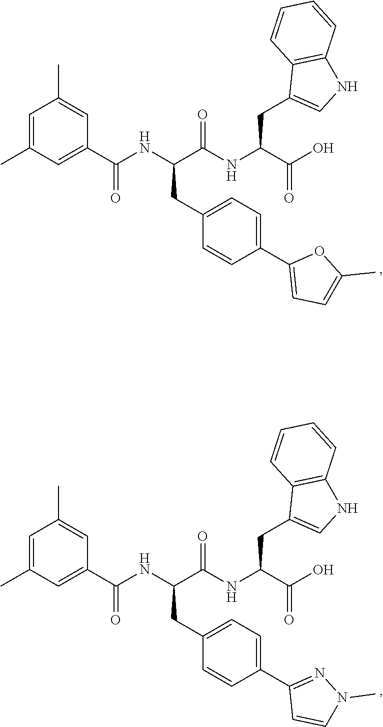
a stereoisomer thereof, a deuterated analog thereof, a fluorinated analog thereof, and a pharmaceutically acceptable salt or solvate thereof.

35. A compound of Formula (6): 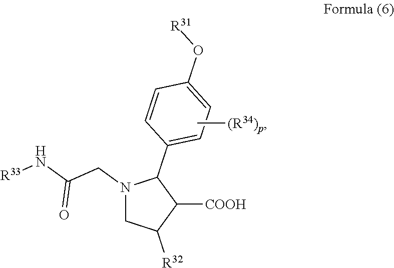
or a pharmaceutically acceptable salt or solvate thereof,
wherein,
R³¹ is substituted or unsubstituted C₁-C₆ alkyl, substituted or unsubstituted C₃-C₈ cycloalkyl, or substituted or unsubstituted C₂-C₇ heterocycloalkyl; wherein if R³¹ is substituted then it is substituted with 1, 2, or 3 substituents independently selected from fluoro, hydroxy, amino, —NH(C₁-C₄ alkyl), —N(C₁-C₄ alkyl)₂, nitro, cyano, C₁-C₄ alkyl, and C₁-C₄ alkoxy;
R³² is substituted or unsubstituted cycloalkyl, substituted or unsubstituted heterocycloalkyl, substituted or unsubstituted aryl, or substituted or unsubstituted heteroaryl;
R³³ is substituted or unsubstituted cycloalkyl, substituted or unsubstituted heterocycloalkyl, substituted or unsubstituted aryl, substituted or unsubstituted heteroaryl, or —CH(CR³⁵)₂, wherein each R³⁵ is independently substituted or unsubstituted C₁-C₆ alkyl, substituted or unsubstituted C₃-C₈ cycloalkyl, substituted or unsubstituted C₂-C₇ heterocycloalkyl, substituted or unsubstituted aryl, or substituted or unsubstituted heteroaryl; wherein if R³³ or R³⁵ is substituted then it is substituted with 1, 2, or 3 substituents independently selected from fluoro, hydroxy, amino, —NH(C₁-C₄ alkyl), —N(C₁-C₄ alkyl)₂, nitro, cyano, C₁-C₄ alkyl, and C₁-C₄ alkoxy;
each R³⁴ is independently deuterium, halogen, hydroxy, amino, nitro, cyano, C₁-C₆ alkyl, C₂-C₆ alkenyl, C₂-C₆ alkynyl, C₃-C₈ cycloalkyl, C₁-C₆ alkoxy, C₁-C₆ haloalkyl, aryl, or heteroaryl; and
p is an integer from 0-4.

36. The compound of claim 35, wherein the compound is selected from the group consisting of: 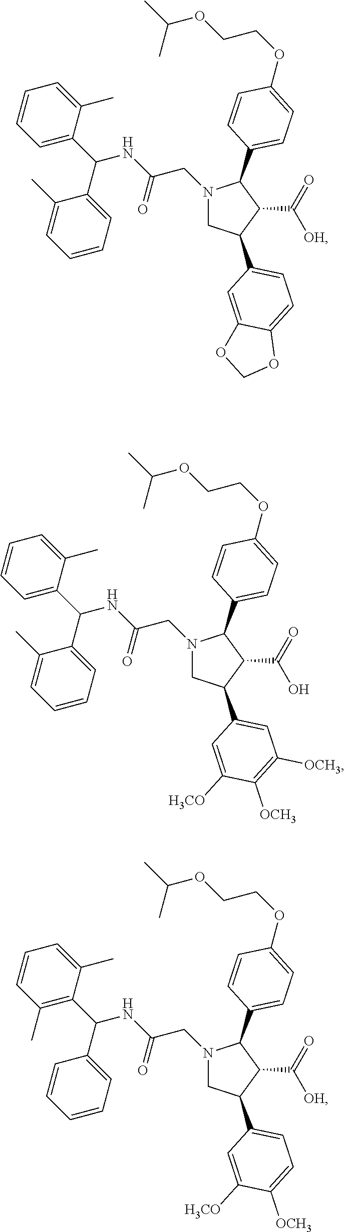 [SMILES: Cc1ccccc1C(NC(=O)CN1C[C@H](c2ccc3c(c2)OCO3)[C@@H](C(=O)O)[C@@H]1c1ccc(OCCOC(C)C)cc1)c1ccccc1C ; COc1cc([C@H]2CN(CC(=O)NC(c3ccccc3C)c3ccccc3C)[C@@H](c3ccc(OCCOC(C)C)cc3)[C@@H]2C(=O)O)cc(OC)c1OC ; COc1ccc([C@H]2CN(CC(=O)NC(c3ccccc3)c3c(C)cccc3C)[C@@H](c3ccc(OCCOC(C)C)cc3)[C@@H]2C(=O)O)cc1OC] 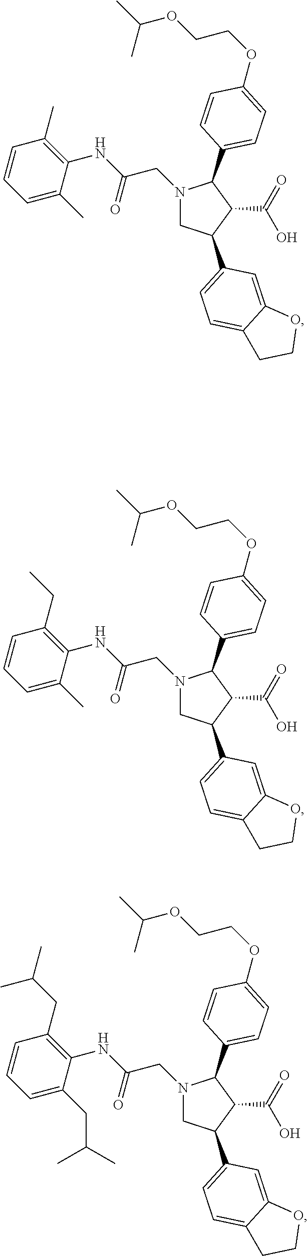 [SMILES: Cc1cccc(C)c1NC(=O)CN1C[C@H](c2ccc3c(c2)OCC3)[C@@H](C(=O)O)[C@@H]1c1ccc(OCCOC(C)C)cc1 ; CCc1cccc(C)c1NC(=O)CN1C[C@H](c2ccc3c(c2)OCC3)[C@@H](C(=O)O)[C@@H]1c1ccc(OCCOC(C)C)cc1 ; CC(C)Cc1cccc(CC(C)C)c1NC(=O)CN1C[C@H](c2ccc3c(c2)OCC3)[C@@H](C(=O)O)[C@@H]1c1ccc(OCCOC(C)C)cc1] 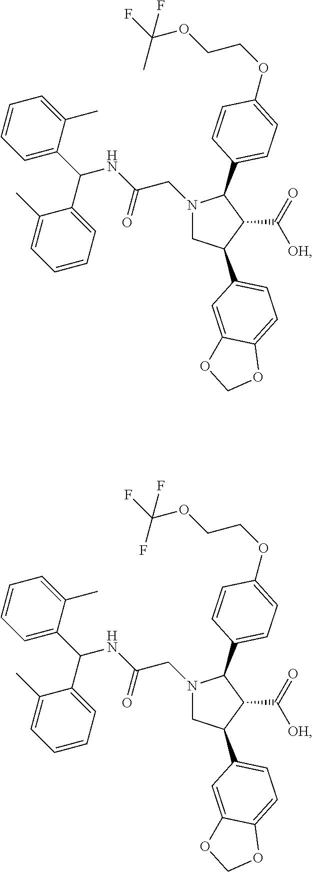 [SMILES: Cc1ccccc1C(NC(=O)CN1C[C@H](c2ccc3c(c2)OCO3)[C@@H](C(=O)O)[C@@H]1c1ccc(OCCOC(C)(F)F)cc1)c1ccccc1C ; Cc1ccccc1C(NC(=O)CN1C[C@H](c2ccc3c(c2)OCO3)[C@@H](C(=O)O)[C@@H]1c1ccc(OCCOC(F)(F)F)cc1)c1ccccc1C]
a stereoisomer thereof, a deuterated analog thereof, a fluorinated analog thereof, and a pharmaceutically acceptable salt or solvate thereof.

37. A compound of Formula (7): 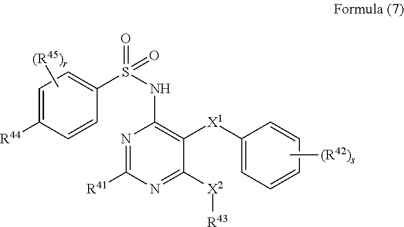
or a pharmaceutically acceptable salt or solvate thereof,
wherein,
R⁴¹ is hydrogen, halogen, —N(R⁴⁶)₂, —COOR⁴⁶, substituted or unsubstituted C₁-C₆ alkyl, substituted or unsubstituted C₃-C₈ cycloalkyl, substituted or unsubstituted C₂-C₇ heterocycloalkyl, substituted or unsubstituted aryl, or substituted or unsubstituted heteroaryl; wherein if R⁴¹ is substituted then it is substituted with 1, 2, or 3 substituents independently selected from fluoro, hydroxy, amino, —NH(C₁-C₄ alkyl), —N(C₁-C₄ alkyl)₂, nitro, cyano, C₁-C₄ alkyl, and C₁-C₄ alkoxy;
each X¹ and X² is independently —O—, —S—, —NR⁴⁶—, —CH₂—, or —(C═O)—;
each R⁴² and R⁴⁵ is independently deuterium, halogen, hydroxy, amino, nitro, cyano, C₁-C₆ alkyl, C₁-C₆ alkoxy, or C₁-C₆ haloalkyl;
R⁴⁴ is hydrogen, halogen, substituted or unsubstituted C₁-C₆ alkyl, substituted or unsubstituted C₁-C₆ fluoroalkyl, or substituted or unsubstituted C₁-C₆ alkoxy;
R⁴³ is substituted or unsubstituted C₁-C₆ alkyl, substituted or unsubstituted C₃-C₈ cycloalkyl, or substituted or unsubstituted C₂-C₇ heterocycloalkyl; wherein if R⁴³ is substituted then it is substituted with 1, 2, or 3 substituents independently selected from fluoro, hydroxy, amino, nitro, cyano, —N(R⁴⁶)₂, —COOR⁴⁶, —C(═O)R⁴⁶, —C(═O)NH(C₁-C₆ alkyl), —C(═O)NH(substituted or unsubstituted aryl), C₁-C₄ alkyl, C₁-C₄ alkoxy, C₂-C₆ alkenyl, C₂-C₆ alkynyl, substituted or unsubstituted C₃-C₈ cycloalkyl, substituted or unsubstituted C₂-C₇ heterocycloalkyl, substituted or unsubstituted aryl, and substituted or unsubstituted heteroaryl;
each R⁴⁶ is independently hydrogen or C₁-C₆ alkyl;
r is an integer from 0-4; and
s is an integer from 0-4.

38. The compound of claim 37, wherein the compound is selected from the group consisting of: 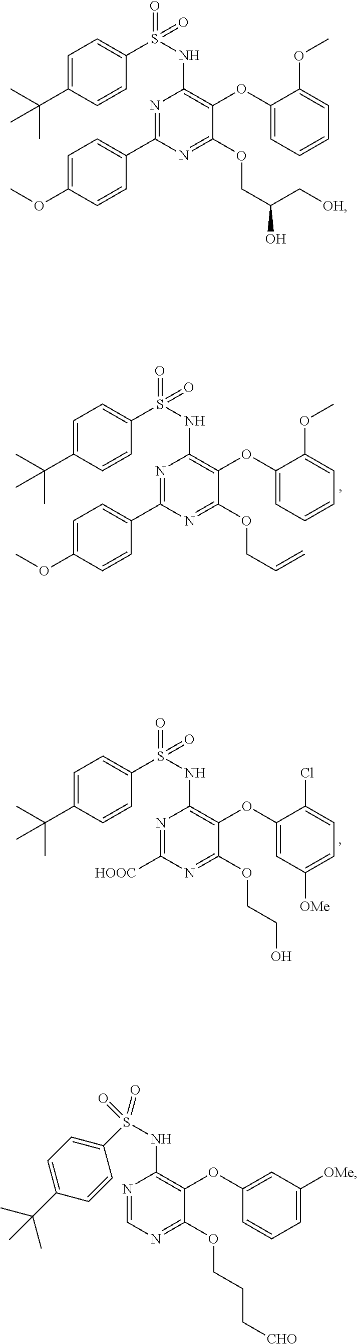 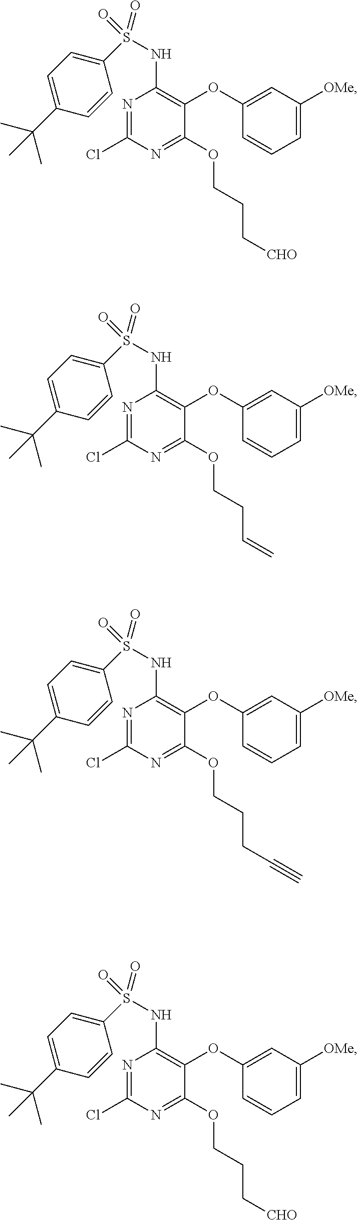 [SMILES: COc1cccc(Oc2c(NS(=O)(=O)c3ccc(C(C)(C)C)cc3)nc(Cl)nc2OCCCC=O)c1 ; C=CCCOc1nc(Cl)nc(NS(=O)(=O)c2ccc(C(C)(C)C)cc2)c1Oc1cccc(OC)c1 ; C#CCCCOc1nc(Cl)nc(NS(=O)(=O)c2ccc(C(C)(C)C)cc2)c1Oc1cccc(OC)c1 ; COc1cccc(Oc2c(NS(=O)(=O)c3ccc(C(C)(C)C)cc3)nc(Cl)nc2OCCCC=O)c1] 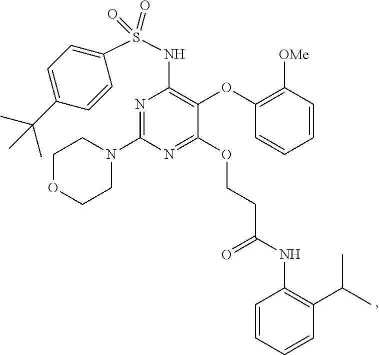 [SMILES: COc1ccccc1Oc1c(NS(=O)(=O)c2ccc(C(C)(C)C)cc2)nc(N2CCOCC2)nc1OCCC(=O)Nc1ccccc1C(C)C]
a stereoisomer thereof, a deuterated analog thereof, a fluorinated analog thereof, and a pharmaceutically acceptable salt or solvate thereof.

39. A compound of Formula (8): 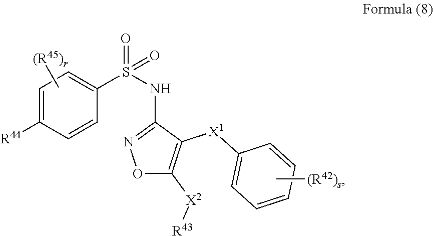
or a pharmaceutically acceptable salt or solvate thereof,
wherein,
each X¹ and X² is independently —O—, —S—, —NR⁴⁶—, —CH₂—, or —(C═O)—;
each R⁴² and R⁴⁵ is independently deuterium, halogen, hydroxy, amino, nitro, cyano, C₁-C₆ alkyl, C₁-C₆ alkoxy, or C₁-C₆ haloalkyl;
R⁴⁴ is hydrogen, halogen, substituted or unsubstituted C₁-C₆ alkyl, substituted or unsubstituted C₁-C₆ fluoroalkyl, or substituted or unsubstituted C₁-C₆ alkoxy;
R⁴³ is substituted or unsubstituted C₁-C₆ alkyl, substituted or unsubstituted C₃-C₈ cycloalkyl, or substituted or unsubstituted C₂-C₇ heterocycloalkyl; wherein if R⁴³ is substituted then it is substituted with 1, 2, or 3 substituents independently selected from fluoro, hydroxy, amino, nitro, cyano, —N(R⁴⁶)₂, —COOR⁴⁶, —C(═O)R⁴⁶, —C(═O)NH(C₁-C₆ alkyl), —C(═O)NH(substituted or unsubstituted aryl), C₁-C₄ alkyl, C₁-C₄ alkoxy, C₂-C₆ alkenyl, C₂-C₆ alkynyl, substituted or unsubstituted C₃-C₈ cycloalkyl, substituted or unsubstituted C₂-C₇ heterocycloalkyl, substituted or unsubstituted aryl, and substituted or unsubstituted heteroaryl;
each R⁴⁶ is independently hydrogen or C₁-C₆ alkyl;
r is an integer from 0-4; and
s is an integer from 0-4.

40. The compound of claim 39, wherein the compound is selected from the group consisting of: 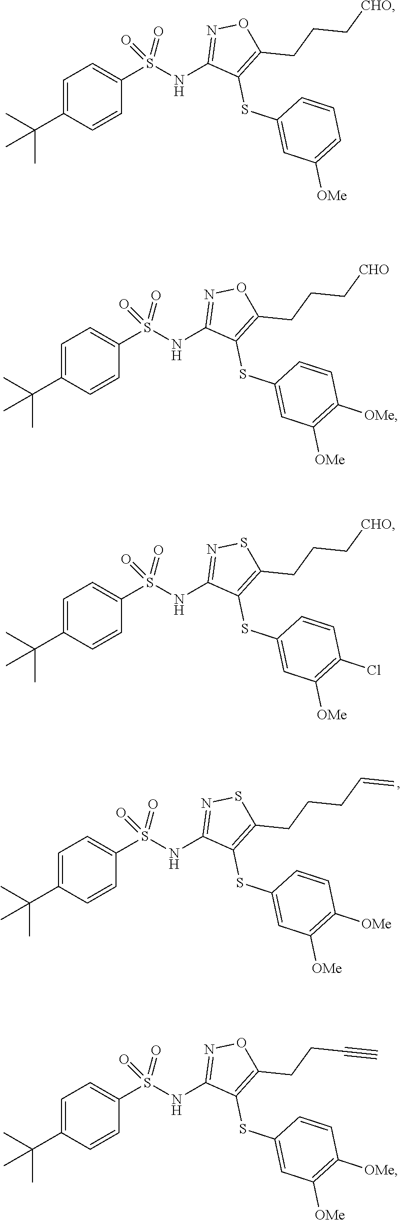
a stereoisomer thereof, a deuterated analog thereof, a fluorinated analog thereof, and a pharmaceutically acceptable salt or solvate thereof.

41. A compound of Formula (9): 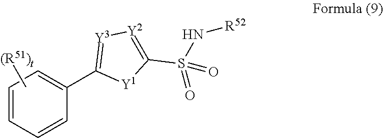
or a pharmaceutically acceptable salt or solvate thereof,
wherein,
each R⁵¹ is independently deuterium, halogen, hydroxy, nitro, cyano, —N(R⁵³)₂, —C(═O)R⁵³, —COOR⁵³, —C(═O)NHR⁵³, substituted or unsubstituted C₁-C₆ alkyl, substituted or unsubstituted C₁-C₆ alkoxy, substituted or unsubstituted C₁-C₆ haloalkyl, substituted or unsubstituted C₃-C₈ cycloalkyl, substituted or unsubstituted C₂-C₇ heterocycloalkyl, substituted or unsubstituted aryl, or substituted or unsubstituted heteroaryl; wherein if R⁵¹ is substituted then it is substituted with 1, 2, or 3 substituents independently selected from halogen, hydroxy, amino, —NH(C₁-C₄ alkyl), —N(C₁-C₄ alkyl)₂, nitro, cyano, C₁-C₄ alkyl, and C₁-C₄ alkoxy;
Y¹ is —O—, —S—, —NR⁵³—;
each Y² and Y³ is independently N or —CR⁵³—;
R⁵² is substituted or unsubstituted cycloalkyl, substituted or unsubstituted heterocycloalkyl, substituted or unsubstituted aryl, or substituted or unsubstituted heteroaryl; wherein if R⁵² is substituted then it is substituted with 1, 2, or 3 substituents independently selected from halogen, hydroxy, amino, —NH(C₁-C₄ alkyl), —N(C₁-C₄ alkyl)₂, nitro, cyano, C₁-C₄ alkyl, and C₁-C₄ alkoxy;
each R⁵³ is independently hydrogen, halogen, hydroxy, nitro, cyano, amino, C₁-C₆ alkyl, or C₁-C₄ alkoxy; and
t is an integer from 0-5.

42. The compound of claim 41, wherein the compound is selected from the group consisting of: 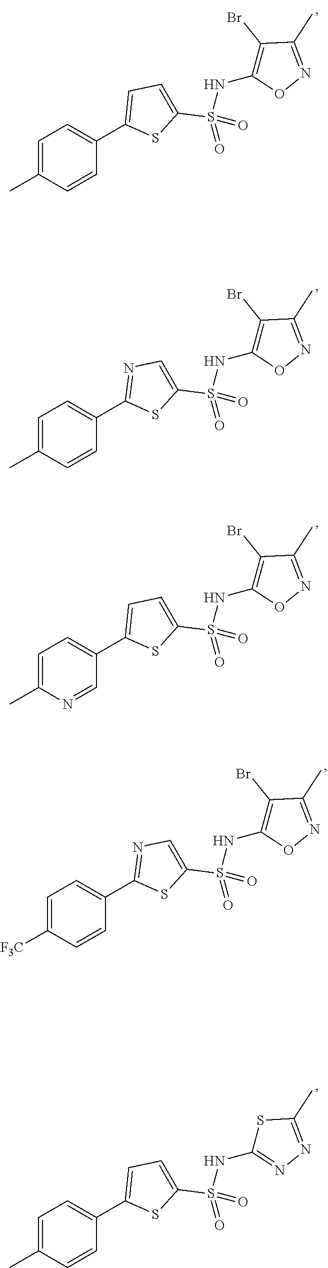 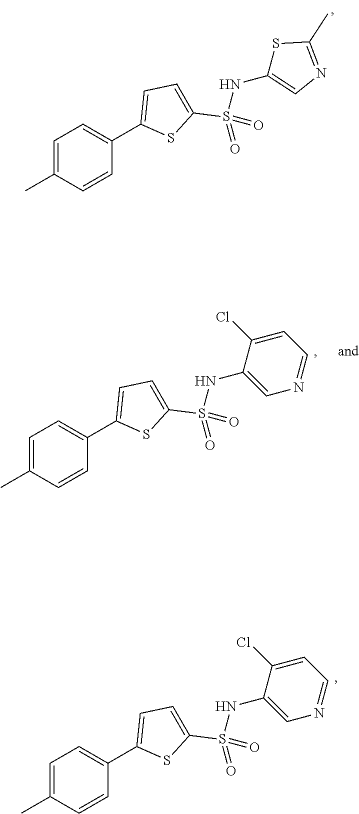
a stereoisomer thereof, a deuterated analog thereof, a fluorinated analog thereof, and a pharmaceutically acceptable salt or solvate thereof.

43. The compound of any one of claims 27-42, wherein the compound is not deuterated.

44. The compound of any one of claims 27-43, wherein the compound is in a form of nanoparticles.

45. A pharmaceutical composition comprising: a compound of any one of claims 27-44 and a pharmaceutically acceptable excipient, diluent, or carrier.

46. The pharmaceutical composition of claim 45, wherein the pharmaceutical composition comprises the pharmaceutically acceptable carrier that is dimethyl sulfoxide.

47. The pharmaceutical composition of claim 45 or 46, wherein the pharmaceutical composition is a nanoparticle formulation.

48. A method of treating cancer in a subject in need thereof, comprising administering to the subject a pharmaceutical composition of any one of claims 45-47.

49. The method of claim 48, further comprising administering an immune checkpoint inhibitor to the subject.

50. The method of claim 49, wherein the immune checkpoint inhibitor is an anti-PD1, or an anti-CTLA-4 agent.

51. The method of claim 49 or 50, wherein the immune checkpoint inhibitor is an antibody.

52. A method of forming a tertiary lymphoid organ (TLO) within a tumor in a subject in need thereof, comprising administering to the subject a compound of any one of claims 27-44 or a pharmaceutical composition of any one of claims 45-47.

53. The method of claim 52, wherein the tertiary lymphoid organ is formed within or adjacent to a tumor.

54. The method of claim 53, wherein the tumor is a solid tumor, melanoma tumor, solid tumor cancer, malignant melanoma, metastatic melanoma, malignant squamous cell carcinoma, metastatic squamous cell carcinoma, glioblastoma, brain cancer, pancreatic cancer, colon cancer, breast cancer, ovarian cancer, prostate cancer, or any combination thereof.

55. A method of treating a cancer in a subject in need thereof, comprising administering to the subject a compound of any one of claims 27-44 or a pharmaceutical composition of any one of claims 45-47, wherein the compound is in an amount effective for treating or ameliorating at least one symptom of the cancer in the subject.

56. The method of claim 55, wherein the cancer is a solid tumor cancer, malignant melanoma, metastatic melanoma, malignant squamous cell carcinoma, metastatic squamous cell carcinoma, glioblastoma, brain cancer, pancreatic cancer, colon cancer, breast cancer, ovarian cancer, prostate cancer, or any combination thereof.

57. The method of any one of claims 52-56, further comprising administering to the subject an additional anti-oncologic therapeutic agent.

58. The method of claim 57, wherein the additional anti-oncologic agent comprises a bRAF inhibitor, an immune checkpoint inhibitor, a caspase-8 inhibitor, an ETAR antagonist, niacinamide, a chemotherapeutic agent, or any combination thereof.

59. The method of claim 58, wherein the additional anti-oncologic agent comprises the immune checkpoint inhibitor.

60. The method of claim 59, wherein the immune checkpoint inhibitor comprises at least one anti-PD1 antibody, at least one anti-PD-L1 antibody, at least one anti-CTLA4 antibody, or any combination thereof.

61. The method of claim 60, wherein the at least one anti-PD1 antibody comprises pidilizumab, BMS-936559, nivolumab, pembrolizumab, or any combination thereof.

62. The method of claim 60, wherein the at least one anti-PD-L1 antibody comprises atezolizumab, avelumab, durvalumab, MDX-1105, or any combination thereof.

63. The method of any one of claims 57-62, wherein the ETBR antagonist and the additional anti-oncologic therapeutic agent are administered at different times.

64. The method of claim 63, wherein the ETBR antagonist is administered 2, 3, 4, or 5 times of the frequency as the additional anti-oncologic therapeutic agent.

65. The method of claim 64, wherein the ETBR antagonist is administered 3 times frequently as the additional anti-oncologic therapeutic agent.

66. The method of claim 65, wherein the compound is administered 3 times every 2-3 weeks and the additional anti-oncologic therapeutic agent is administered 1 time every 2-3 weeks.

67. The method of claim 66, wherein the compound is administered 3 times about every 21 days and the additional anti-oncologic therapeutic agent is administered 1 time about every 21 days.

68. The method of any one of claims 52-56, wherein the subject is a human.

69. The method of any one of claims 52-68, wherein the subject is resistant to an immunotherapy before the treatment.

70. The method of any one of claims 52-69, wherein the administration restores Tumor Infiltrating Lymphocytes (TILs), intratumoral tertiary lymphoid organ (TLO) formation, or a combination thereof, in a tumor microenvironment.
